(19) United States
(12) Patent Application Publication
(10) Pub. No.: US 2022/0023345 A1
(43) Pub. Date: Jan. 27, 2022

(54) Compositions Comprising CD34+ Cells and Methods for Repairing a Lung Injury After Severe Virus Infection

(71) Applicant: Caladrius Biosciences, Inc., Basking Ridge, NJ (US)

(72) Inventor: Douglas Losordo, Scarsdale, NY (US)

(73) Assignee: Caladrius Biosciences, Inc., Basking Ridge, NJ (US)

(21) Appl. No.: 17/377,922

(22) Filed: Jul. 16, 2021

Related U.S. Application Data
(60) Provisional application No. 63/055,118, filed on Jul. 22, 2020.

(51) Int. Cl. A61K 35/17; A61K 35/16; A61P 11/00; A61P 31/14; A61K 9/00; A61K 35/15

(52) U.S. Cl. CPC A61K 35/17; A61K 35/16; A61K 35/15; A61P 31/14; A61K 9/0019; A61P 11/00

(57) ABSTRACT

The described invention provides a method for treating a subject at risk for a lung injury derived from a severe virus infection. The steps of the method include (a) receiving a subcutaneous injection of a bone marrow stimulant to mobilize CD34+ cells into the peripheral blood; (b) harvesting CD34+ cells from the peripheral blood by apheresis; (c) selecting CD34+ cells by positive selection; (d) formulating a CLBS119 cell product by suspending the selected CD34+ cells in an isotonic solution with serum ranging from 5% to 40%, inclusive and human serum albumin ranging from 0.5%-10%, inclusive, to form a pharmaceutical composition; and (e) administering the cell product to the subject. The sterile pharmaceutical composition contains a therapeutic amount of a mobilized nonexpanded, isolated population of autologous mononuclear cells enriched for CD34+ cells with a purity ranging from 55% to 100%, inclusive, which further contains a subpopulation of potent CD34+/CXCR4+ cells. The mobilized nonexpanded, isolated population of autologous mononuclear cells enriched for CD34+ cells with a purity ranging from 55% to 100%, inclusive, which further contains a subpopulation of potent CD34+/CXCR4+ cells when tested in vitro after passage through an infusion catheter after acquisition: (i) has CXCR-4 mediated chemotactic activity and moves in response to SDF-1; (ii) can form hematopoietic colonies; and (iii) is at least 80% viable. According to some embodiments, the severe virus infection is caused by influenza or a human coronavirus.

### CROSS-REFERENCE TO RELATED APPLICATIONS

This application claims priority to and benefit of U.S. Provisional Patent Application No. 63/055,118, filed Jul. 22, 2020, the disclosure of which is incorporated herein by reference in its entirety.

### FIELD OF THE INVENTION

The described invention relates to compositions and methods for treating a lung injury after a severe virus infection in a subject at risk.

### BACKGROUND OF THE INVENTION

Normal Lung Structure/Function

The normal lung is structured to facilitate carbon dioxide excretion and oxygen transfer across the distal alveolar-capillary unit. The selective barrier to fluid and solutes in the uninjured lung is established by a single-layer lining of endothelial cells linked by plasma membrane structures, including adherens and tight junctions [Matthay, M A et al., Nature Revs. (2019) 5: 18, citing Bhattacharya, J., & Matthay, M A. Annu. Rev. Physiol. (2013) 75: 593-615]. The vast surface of the alveolar epithelium is lined by flat alveolar type I (ATI) cells along with cuboidal shaped alveolar type II (ATII) cells, forming a very tight barrier that restricts even the passage of small solutes but allows diffusion of carbon dioxide and oxygen. The ATII cells secrete surfactant, the critical factor that reduces surface tension, enabling the alveoli to remain open and facilitating gas exchange. Both ATI and ATII cells have the capacity to absorb excess fluid from the airspaces by vectorial ion transport, primarily by apical sodium channels and basolateral Na+/K+-ATPase pumps [Id., citing Matthay, M A. Am. J. Respir. Crit. Care Med. (2014) 189: 1301-8]. Thus, when alveolar edema develops, reabsorption of the edematous fluid depends on junctions between ATI and ATII cells and intact ion transport channels in the epithelial cells. Once the edematous fluid is absorbed into the lung interstitium, the fluid can be removed primarily by lymphatics and the lung microcirculation. The cellular makeup of the normal alveolus includes alveolar macrophages but not polymorphonuclear leukocytes (neutrophils), although they can be rapidly recruited from the circulation. Alveolar macrophages, neutrophils and other immune effector cells, including monocytes and platelets, are critical in defense of the normal lung and have key activities in acute lung injury [Matthay, M A et al., Nature Revs. (2019) 5: 18].

Pulmonary circulation.

The pulmonary circulation begins at the pulmonary valve, marking the vascular exit from the right side of the heart, and extends to the orifices of the pulmonary veins in the wall of the left atrium, which marks the entrance into the left side of the heart. The pulmonary circulation includes the pulmonary trunk (also called the “right ventricular outflow tract”), the right and left main pulmonary arteries and their lobar branches, intrapulmonary arteries, large elastic arteries, small muscular arteries, arterioles, capillaries, venules, and large pulmonary veins. Because of this heterogeneity and differences in physiologic behavior, the vessels of the pulmonary circulation are subdivided on a functional basis into extra-alveolar vessels and alveolar vessels. In addition, the small vessels that participate in liquid and solute exchange are often collectively termed the “pulmonary microcirculation.” The anatomic boundaries of the extra-alveolar and alveolar vessels and the microcirculation are undefined and likely depend on conditions such as lung volume and levels of intrapleural and interstitial pressures [Garcia, J G N., in Murray and Nadel's Textbook of Respiratory Medicine (6th Ed.), V. Courtney Broaddus, Joel Ernst, Talmadge E King, Jr, Stephen C. Lazarus, John F. Murray, Jay A. Nadel, Arthur S. Slutsky, Michael B. Gotway, Eds., Elsevier (2016) Chapter 6, pp. 92-110].

Beyond its role in gas exchange, the pulmonary circulation has important additional functions. The microvessels exchange solutes and water, and the mechanisms regulating the balance of fluid and solutes in extravascular spaces of the lung are critical to the understanding of the pathophysiology of pulmonary edema [Matthay, M A & Murray, J F in Murray and Nadel's Textbook of Respiratory Medicine (6th Ed.), V. Courtney Broaddus, Joel Ernst, Talmadge E King, Jr, Stephen C. Lazarus, John F. Murray, Jay A. Nadel, Arthur S. Slutsky, Michael B. Gotway, Eds., Elsevier (2016) Chapter 62, 1096-1117].

Increases in lung vascular permeability are operationally defined in the Starling equation by an increased capillary filtration coefficient (LpS), which indicates decreased resistance to water flow across the capillary wall barrier, and a decreased albumin reflection coefficient (σalb), which describes the albumin permeability of the vascular endothelial barrier. The critical functional definition of increased lung vascular permeability is the extravasation of protein-rich fluid into the interstitial space and ultimately into the alveolar space, resulting in fulminant pulmonary edema. In high-permeability pulmonary edema, the alveolar fluid protein concentration approximates the plasma protein concentration, whereas in hydrostatic edema (i.e., edema resulting from increase in the pulmonary capillary hydrostatic pressure), the ratio of plasma to alveolar fluid protein concentration is usually less than 0.6. [Garcia, J G N., in Murray and Nadel's Textbook of Respiratory Medicine (6th Ed.), V. Courtney Broaddus, Joel Ernst, Talmadge E King, Jr, Stephen C. Lazarus, John F. Murray, Jay A. Nadel, Arthur S. Slutsky, Michael B. Gotway, E ds., Elsevier (2016) Chapter 6, pp. 92-110].

Starling's original model of semi-permeable capillaries subject to hydrostatic and oncotic pressure gradients within the extracellular fluid was derived from experiments injecting serum or saline solution into the hindlimb of a dog. Starling deduced that the capillaries and post-capillary venules behave as semi-permeable membranes absorbing fluid from the interstitial space [Woodcock, T E, Woodcock, T M. Britich J. Anaethesia (212) 108 (3): 384-94]. The revised Starling equation based on recent research considers the contributions of the endothelial glycocalyx layer (EGL), the endothelial basement membrane, and the extracellular matrix. Transvascular fluid exchange depends on a balance between hydrostatic and oncotic pressure gradients. Fluid is filtered to the interstitial space under a dominant hydrostatic pressure gradient (capillary pressure Pc minus ISF pressure Pis) at the arteriolar portion of capillaries, and it was believed that it is absorbed back under a dominant colloid osmotic pressure (COP) gradient (capillary COP πc minus ISF COP πis) at the venular end. The effect of πis on transvascular fluid exchange was shown to be much less than predicted by the standard Starling equation [Id., citing Adamson, R H et al. J. Physiol. (2004) 557: 889-907], which therefore has to be revised [Id., citing Levick, J R. J. Physiol. (2004) 557: 704] It is now established that non-fenestrated capillaries normally filter fluid to the ISF throughout their length. Absorption through venous capillaries and venules does not occur. πc opposes, but does not reverse, filtration; most of the filtered fluid returns to the circulation as lymph; and the EGL excludes larger molecules and occupies a substantial volume of the intravascular space

While bacteria (e.g., Streptococcus pneumoniae) are a major cause of lower respiratory tract infections, the pathogens that most often cause acute respiratory infections are viruses. Respiratory viral infections are an important cause of morbidity and, in some settings, of mortality. One important feature of respiratory viral infections is the nonspecific nature of clinical signs and symptoms.

Influenza Viruses

Much of what is known about virus-induced lung injury comes from influenza virus studies. Influenza viruses are a prime example of pathogens that have epidemic or pandemic potential and that have previously posed a public health risk. There are three genera of influenza viruses: influenza virus A, influenza virus B, and influenza virus C. The influenza viruses, especially influenza virus A, are considered the most variable of the respiratory viruses. Influenza A viruses are subtyped based on their two surface antigens: hemagglutinin (HA; H1-H16) and neuraminidase (NA; N1-N9), which are responsible for host receptor binding/cell entry and cleavage of the HA-receptor complex to release newly formed viruses, respectively. Aquatic birds are the natural reservoir of influenza A viruses, harboring all possible subtypes [McNamara, P S, Van Doom, H R, Respiratory viruses and atypical bacteria. In Manson's Tropical Infectious Diseases (23rd Ed.) (2014), 215-224].

Both influenza virus A and B exhibit antigenic drift. This phenomenon occurs when the surface antigens of the virus gradually change, progressively and directionally, to escape immunological pressure from the host species. Yearly epidemics of influenza virus A and B are caused worldwide by these drift variants, and contribute to mortality (an estimate 250,000-500,000 every year) in the elderly, and in those with pre-existing conditions, such as chronic cardiopulmonary or renal disease; diabetes, immunosuppression, or severe anemia. New lineages of influenza virus A emerge every few decades through re-assoiintent of gene segments in animal hosts infected with two different viruses (antigenic shift), resulting in global pandemics with varying severity due to the absence of immunity in the human population (e.g., 1918 Spanish flu: H1N1, 40-100 million deaths; 1957 Asian flu: H2N2, 2 million deaths; 1968 Hong Kong flu: H3N2, 500,000 deaths; 2009 H1N1-pdm09, 15,000 deaths). Sporadic dead-end human infections of animal (especially avian) viruses are known to occur and have caused concern regarding pandemic potential. Highly pathogenic H5N1 viruses were first detected in birds in 1996 in China. In 2003, the virus re-emerged in China. Since then it has become panzootic among poultry and wild birds. The disease presents as a rapidly progressive viral pneumonia with severe leucopenia and lymphopenia, progressing to acute respiratory distress syndrome (ARDS) and multi-organ dysfunction. In 2013, another avian influenza virus (H7N9) caused zoonotic transmission events to humans in China, with no recorded sustained human-to-human transmission. The case fatality rate was around 20% and the elderly were most affected[Id]

Highly pathogenic avian H5N1 influenza viruses preferentially infect alveolar type II pneumocytes in human lung [Weinheimer, V K., et al. J. Infect. Dis. (2012) 206 (11): 1685-94].

The development of severe influenza reflects a combination of pathologic processes, including the spread of viral infection from the upper to the lower respiratory tract, bacterial superinfection of injured mucosal surfaces, and the effect of host inflammatory responses on pulmonary function [Armstrong, S M et al. Antiviral Res. (2013) 99: 113-118]. The inflammatory response to infection is defined primarily by altered vascular function, in which the endothelial barrier opens to permit the passage of immune cells, antibody and complement molecules and other substances from the blood stream into the tissues. In the lungs, this standard response to injury results in the shift of fluid into alveolar species, which, in the case of severe infection, may lead to progressive respiratory compromise (acute respiratory distress syndrome, ARDS) [Armstrong, S M et al. Antiviral Res. (2013) 99: 113-118].

Coronavirus

Coronaviruses (CoVs), a large family of single-stranded RNA viruses, can infect a wide variety of animals, including humans, causing respiratory, enteric, hepatic and neurological diseases [Yin, Y., Wunderink, R G, Respirology (2018) 23 (2): 130-37, citing Weiss, S R, Leibowitz, I L, Coronavirus pathogenesis. Adv. Virus Res. (2011) 81: 85-164]. Human coronaviruses, which were considered to be relatively harmless respiratory pathogens in the past, have now received worldwide attention as important pathogens in respiratory tract infection. As the largest known RNA viruses, CoVs are further divided into four genera: alpha-, beta-, gamma- and delta-coronavirus.

Coronaviruses are enveloped with a non-segmented, positive sense, single strand RNA, with size ranging from 26,000 to 37,000 bases; this is the largest known genome among RNA viruses [Yang, Y. et al., J. Autoimmunity (2020) doi.org/10.1016/j.jaut.2020.102434, citing Weiss, S R et al. Microbiol. Mol. Biol. Rev. (2005) 69 (4): 635-64]. The viral RNA encodes structural proteins, and genes interspersed with in the structural genes, some of which play important roles in viral pathogenesis [Yang, Y. et al., J. Autoimmunity (2020) doi.org/10.1016/j.jaut.2020.102434, citing Fehr, A R, Perlman, S. Methods Mol. Biol. (2015) 1282: 1-23; Zhao, L. et al. Cell Host Microbe (2012) 11(6): 607-16]. The spike protein (S) is responsible for receptor binding and subsequent viral entry into host cells; it consists of Si and S2 subunits. The membrane (M) and envelope (E) proteins play important roles in viral assembly; the E protein is required for pathogenesis [Id., citing DeDiego, M L, et al. J. Virol. (2007) 81(4): 1701-13; Nieto-Torres, J L et al. PLoS Pathog. (2014) 10(5): e1004077]. The nucleocapsid (N) protein contains two domains, both of which can bind virus RNA genomes via different mechanisms, and is necessary for RNA synthesis and packaging the encapsulated genome into virions. [Yang, Y. et al., J. Autoimmunity (2020) doi.org/10.1016/j.jaut.2020.102434., citing Fehr, A R, Perlman, S. Methods Mol. Biol. (2015) 1282: 1-23; Song, Z. et al. Viruses (2019) 11(1): 59; Chang, C K et al., J. Biomed. Sci. (2006) 13(1): 59-72; Hurst, K R, et al. J. Virol. (2009) 83 (14): 7221-34]. The N protein also is an antagonist of interferon and viral encoded repressor (VSR) of RNA interference (RNAi), which benefits viral replication [Id., citing Cui, L. et al. J. Virol. (2015) 89 (17): 9029-43].

Before December 2019, six coronavirus species had been identified to infect humans and cause disease. Among them, 229E, OC43, NL63, and HKU1 infections are frequently mild, mostly caused common cold symptoms [Xu, X. et al. Eur. J. Nuclear Medicine & Molec. Imaging (2020) doi.org/10.1007/s00259-020-04735-9, citing Su, S. et al. Trends Microbiol. (2016) 24: 490-502]. The other two species, severe acute respiratory syndrome coronavirus (SARS-CoV) and Middle East respiratory syndrome coronavirus (MERS-CoV), have a different pathogenicity and have caused fatal illness [Id., citing Cui, J. et al. Nat. Rev. Microbio. (2019) 17: 181-92].

SARS-CoV-2 is the seventh member of the coronaviruses that infects humans [Zhu, N. et al. N. Engl. J. Med. (2020) 382: 727-33].

Beginning in December 2019, pneumonia cases of unknown origin were identified in Wuhan, China. The cause has been identified as severe acute respiratory syndrome coronavirus 2 (SARS-CoV-2) and the virus-infected pneumonia was later designated coronavirus disease 2019 (COVID-19) by WHO. Due to efficient person-to-person transmission, SARS-CoV-2 has resulted in a pandemic that is still evolving. The extent of the disease, its epidemiology, pathophysiology and clinical manifestations are being documented on an ongoing basis [Guan w. et al. N. Engl. J. Med. (2020) 382: 1708-20; Yang, Y. et al., J. Autoimmunity (2020) doi.org/10.1016/j.jaut.2020.102434].

Susceptible Patient Populations

Infected patients predominantly presented with fever, cough, and radiological ground glass lung opacities, which resemble SARS-CoV and MERS-CoV infections [Id., citing Huang, C. et al. Lancet (2020) 395: 497-506]. The absence of fever in COVID-19 is more frequent than in SARS-CoV (1%) and MERS-CoV infection (2%), so afebrile patients may be missed if the surveillance case definition focuses on fever detection. [Guan, W., et al. New Engl. J. Med. (2020) 382: 1708-2]0. Some patients with SARS-CoV-2 infection are asymptomatic, while in severe cases, acute respiratory distress syndrome, septic shock, difficult to correct metabolic acidosis and coagulation dysfunction develop rapidly [Pan, Y. et al. European Radiol. (2020) doi.org/10.1007/s00330-020-06731-x].

Lung imaging pathology, e.g., the number of affected lobes, the presence of ground glass nodules, patchy/punctate ground glass opacities, patchy consolidation, fibrous stripes and irregular solid nodules by CT, manifests earlier than clinical symptoms [Id.]. It was found that as the disease progressed, the range of ground glass density patches and consolidation increased, which were mainly distributed in the middle and outer zones of the lung. When a patient's condition improved, a little fibrous stripe may appear. When a patient's condition worsened, the lungs showed diffuse lesions, and the density of both lungs increased widely, showing a “white lung” appearance, which seriously affects lung function [Id].

Most patients who have died from SARS-CoV-2 had other chronic medical conditions, were elderly patients or were immunocompromised. One study reported that most SARS-CoV-2 infected patients at the China outbreak epicenter in Wuhan were >50 years of age; the mean age was much older than patients infected with H1N1 or with Middle East respiratory syndrome (MERS [Xie, J. et al., Intensive Care Med. Doi.10.1.1007/s00134-020-05979-7, citing Dominguez-Cherit, G. et al. JAMA (2009) 302 (17): 1880-87; Kumar, A. et al. JAMA (2009) 302 (17): 1872-79; Lu, R., et al. Lancet (2020) doi.org/10.1016/50140-6736(20)30251-8]. About 30-50% had chronic comorbidities. Hypertension (48.2%) was the most common comorbidity in non-surviving patients, followed by diabetes (26.7%) and ischemic heart disease (17.0% similar to data reported by others [Id., citing Chen, N. et al. Lancet (2020) doi.org/10.1016/S0140-6736(20)30211-7; Wang, D. et al. JAMA (2020) doi.org/10.1001/jama.2020.1585]. Of the patients who died only about 25% received invasive mechanical ventilation or extracorporeal membrane oxygenation (ECMO). The mortality of patients who received ECMO was high: of 28 patients who received ECMO, 14 died, 5 weaned successfully and 9 at time of writing were still on ECMO. Lack of ventilators, fear of becoming infected during intubation and unclear need for intubation were the main reasons for delaying invasive ventilation. Duration from initial symptoms to respiratory failure in most patients was >7 days, which is longer than H1N1 [Id., citing Dominguez-Cherit, G. et al. JAMA (2009) 302 (17): 1880-87; Kumar, A. e al. JAMA (2009) 302 (17): 1872-79]. Many patients that went on to develop respiratory failure had hypoxemia, but without signs of respiratory distress, especially in elderly patients (“silent hypoxemia”). Only a very small proportion of patients had other organ dysfunction (e.g., shock, acute kidney injury) prior to developing respiratory failure [Xie, J. et al., Intensive Care Med. doi.10.1.1007/s00134-020-05979-7].

Pathogenesis

COVID-19 infection in the lung results in severe lung damage, which is marked by inflammation, loss of lung endothelial cells/integrity and destruction of the lung microvasculature. It is known from other syndromes characterized by similar acute pathology (e.g., SARS, MERS, ARDS) that the failure to recover endothelial integrity in the lung impairs functional recovery and is associated with ongoing fibrosis, morbidity and mortality. SARS-CoV preferentially infects alveolar type II cells compared to type I cells [Mason, R J. Eur. Respiratory J. (2020) 55: 2000607, citing Mossel, E C et al. Virology (2008) 372: 127-35; Weinheimer, V K, et al. J. Infect. Dis. (2012) 206: 1685-94]. Normally, alveolar type II cells are the precursor cells for alveolar type I cells. The infected alveolar units tend to be peripheral and subpleural [Id., citing Wu, J. et al. Invest. Radiol. (2020) doi.org/10.1097/RLI.0000000000000670; Zhang, S. et al. Eur. J. Respir. J. (2020) In press]. SARS-CoV propagates within type II cells, large number of viral particles are released, and the cells undergo apoptosis and die [Id., citing Qian Z. et al. Am. J. Respir. Cell Mol. Biol. (2013) 48: 742-48]. The released viral particles then infect type II cells in adjacent units.

It has been reported that patients affected by SARS-CoV-2 pneumonia show some common CT imaging features [Xu, X. et al. Eur. J. Nuclear Medicine & Molec. Imaging (2020) doi.org/10.1007/s00259-020-04735-9]. Of 90 patients with laboratory-identified SARS-CoV-2 infection, more than half presented bilateral, multifocal lung lesions, with peripheral distribution, and 59% of patients had more than two lobes involved. Of all included patients, COVID-19 pneumonia presented with ground glass opacities in 65 (72%), consolidation in 12 (13%), crazy paving pattern in 11 (12%), interlobular thickening in 33 (37%), adjacent pleura thickening in 50 (56%), and linear opacities combined in 55 (61%). Pleural effusion, pericardial effusion, and lymphadenopathy were uncommon findings. Baseline chest CT did not show any abnormalities in 21 patients (23%), but 3 patients presented bilateral ground glass opacities on the second CT after 3-4 days.

Angiotensin converting enzyme 2 (ACE2) and dipeptidyl peptidase 4 (DPP4) are known host receptors for SARS-CoV and MERS-CoV respectively [Yang, Y. et al., J. Autoimmunity (2020) doi.org/10.1016/j.jaut.2020.102434, citing Kuhn, R I, et al. Cell Mol. Life Sci. (2004) 61 (21): 2738-43; Raj, V S, et al. Nature (2013) 495 (7440): 251-54].

SARS-CoV-2 also uses ACE2 to gain entry into host cells.

ACE2 is not only highly expressed in lung AT2 cells, esophagus upper and stratified epithelial cells, but also in absorptive enterocytes from the ileum and colon [Yang, Y. et al., J. Autoimmunity (2020) doi.org/10.1016/j.jaut.2020.102434., citing Zhang, H. et al. bioRxiv (2020) 2020.01.30.927806]. Thus, although the respiratory system is a primary target of SARS-CoV-2, bioinformatics analysis of single-cell transcriptosomes datasets of lung, esophagus, gastric, ileum and colon reveal that the digestive system is also a potential route of entry for COVID-19; Cardiovascular complications are rapidly emerging as a key threat in COVID-19. [Varga, Z. et al. The Lancet (2020) doi.org/10.1016/S0140-6736(20)30937-5] Endothelial cell involvement across vascular beds of different organs has been demonstrated in a series of patients with COVID-19. [Varga, Z. et al. The Lancet (2020) doi. org/10.1016/50140-6736(20)30937-5].

The renin angiotensin system (RAS) is a central regulator of renal and cardiovascular function. Classically, it consists of angiotensin converting enzyme (ACE), its product, angiotensin (Ang) II and receptors for Ang II, angiotensin Type 1 (AT₁) and angiotensin type 2 (AT2) receptors. RAS further includes ACE2, a monocarboxypeptidase that generates Ang-(1-7) from Ang II. Angiotensin-(1-7) is an endogenous ligand for the G protein-coupled receptor Mas; Mas therefore mediates the biological actions of Ang-(1-7) [Singh, N. et al. Am J. Physiol. Heart Circ. Hysiol. (2015) 309 (10): H1697-H1707, citing Santos, R A et al. Proc. Nat. Acad. Sci. USA (2003) 100: 8258-63].

Ang II produces hypertensive, pro-oxidative, hypertrophic and pro-fibrotic effects in the cardiovascular system. Ang-(1-7) elicits counter-regulatory effects on the ACE/AngII pathway by reducing vasodilatory, antihypertensive, antihypertrophic, antifibrotic and antithrombotic effects [Id., citing Ferreira, A J, et al. Hypertension (2010) 55: 207-13; Jusuf, D. et al. Eur. J. Pharmacol. (2008) 585: 303-12].

It has been reported that even though the expression of hACE2 in T cells is very low, SARS-CoV-2 can infect T cells through receptor-dependent, S protein-mediated membrane fusion T cells; similar to MERS-CoV, SARS-CoV-2 infection of T cells is abortive [Wang, X. et al. Cellular & Mol. Immunol. (2020) doi.org/10.1038/s41423-020-0424-9.]

SARS-CoV viroporin 3a was reported to trigger the activation of the NLRP3 inflammasome and the secretion of IL-1(3 in bone marrow macrophages, suggesting SARS-CoV induced cell pyroptosis, a novel inflammatory form of programmed cell death [Yang, Y. et al., J. Autoimmunity (2020) doi.org/10.1016/j.jaut.2020.102434, citing Cookson, B T, Brennan, M A. Trends Microbiol. (2001) 9(3): 113-14; Chen, L Y et al. Front. Microbiol. (2019) 10: 50]. Studies have shown that patients infected with SARS-CoV-2 have increased IL-1β in the serum [Id., citing Huang, C. et al. Lancet (2020) 395 (10223): 497-506]. The rise of IL-1β is a downstream indicator of cell pyroptosis [Yang, Y. et al., J. Autoimmunity (2020) doi.org/10.1016/j.jaut.2020.102434] The pathways involved in the activation of signaling between NLRP3m IL-1β, IL-18 and GSDMD are illustrated in FIG. 1 [taken from Yang, Y. et al., J. Autoimmunity (2020) doi.org/10.1016/j.jaut.2020.102434, FIG. 6].

Therapeutics

In vitro, interferons (IFNs) are only partially effective against coronaviruses [Yang, Y. et al., J. Autoimmunity (2020) doi.org/10.1016/j.jaut.2020.102434, citing Cinatl, J. et al. Lancet (2003) 362 (9380): 293-94]. In vivo, the effectiveness of IFNs combined with ribavirin requires further evaluation [Id., citing Stockman, L L J, et al. PLoS Med. (2006) 3(9): e343]. Other new antivirals (e.g., remdecivir) are being developed and tested. A variety of other agents, including antiviral peptides and corticosteroids, have been shown to be effective in vitro and/or in animal models [Id., citing Zumla, A. et al. Nat. Rev. Drug Discov. 2016] 15(5): 327-47; Lee, N. et al. J. Clin. Virol. (2004) 31 (4): 304-9], although clinical evidence does not support the use of corticosteroid treatment for SARS-CoV-2 lung injury [Id., citing Russell, C D et al. The Lancet (2020) 395(10223):473-475]. Vaccines that have been developed to CoVs are either not effective, or in some cases have been reported to be involved in the selection of novel pathogenic CoVs via recombination of circulating strains [Id., citing Fehr, A R, Perlman, S. Methods Mol. Biol. (2015) 1282: 1-23; Zumla, A. e et al., Nat. Rev. Drug Discov. (2016) 15(5): 327-47].

Acute Lung Injury (ALI) and Acute Respiratory Distress Syndrome (ARDS)

Acute lung injury (ALI) and the acute respiratory distress syndrome (ARDS) describe clinical syndromes of acute respiratory failure with substantial morbidity and mortality. A consensus definition that has been widely adopted for both clinical and research purposes requires the acute onset of diffuse bilateral pulmonary infiltrates by chest radiograph; a PaO₂/FiO₂≤300 for ALI and ≤200 for ARDS; and a pulmonary artery wedge pressure (PAWP)≤18 or no clinical evidence of left atrial hypertension [Johnson, E R, and Matthay, M A, J. Aerosol Med. Pulm. Drug Deliv. (2010) 23 (4): 243-52]. Predisposing clinical factors include sepsis, pneumonia, aspiration, trauma, pancreatitis, blood transfusions, and smoke or toxic gas inhalation [Id., citing Ware, L B, Matthay, M A. N. Engl. J. Med. (2000) 342: 1334-49]. Severe sepsis and multiple transfusions are associated with the highest incidence of ARDS; the lowest rates occur in patients with trauma or drug overdoses. [Id., citing Rubenfeld, G D et al. N. Engl. J. Med. (2005) 353: 1685-93; Hudson, L D, Steinberg, K P. Chest (1999) 116: 74S-82S]. The risk for lung injury is higher for patients with multiple comorbidities, chronic alcohol abuse, or chronic lung disease [Id., citing Ware, L B, Matthay, M A. N. Engl. J. Med. (2000) 342: 1334-49].

Acute Lung Injury (ALI)

Acute lung injury is a disorder of acute inflammation that causes disruption of the lung endothelial and epithelial barriers. The alveolar-capillary membrane is comprised of the microvascular endothelium, interstitium, and alveolar epithelium. Cellular characteristics of ALI include loss of alveolar-capillary membrane integrity, excessive transepithelial neutrophil migration, and release of pro-inflammatory, cytotoxic mediators [Id., citing Ware, L B, Matthay, M A. N. Engl. J. Med. (2000) 342: 1334-49; Matthay, M A, Zimmerman, G A. Am. J. Respir. Cell Mol. Biol. (2005) 33: 319-27] Biomarkers found on the epithelium and endothelium and that are involved in the inflammatory and coagulation cascades, such as von Willebrand factor (VWF) antigen [Id., citing Ware, L B et al. Crit. Care med. (2001) 29: 2325-31; Ware, L B et al. Am. J. Respir. Crit. Care Med. (2004) 170: 766-72; Flori, H R et al. Pediatr. Crit. Care Med. (2007) 8: 96-101], intercellular adhesion molecule [ICAM-1; Id., citing Flori, H R et al. Pediatr. Crit. Care Med. (2003) 4: 315-21; McClintock, D. et al. Crit. Care (2008) 12: R41; Calfee, C S et al. Intensive Care Med. (2009) 35: 248-57], surfactant protein D [SP-D, Id., citing Eisner, M D, et al. Thorax (2003) 58: 983-88], receptor for advanced glycation end-products [RAGE, Id., citing Calfee, C S, et a. thorax (2008) 63: 1083-89], IL-6 [Id., citing Meduri, G U et al. Chest (1995) 108: 1303-1314; Parsons, P E et al. Crit. Care Med. (2005) 33: 1-6], IL-8 [Id., citing Meduri, G U et al. Chest (1995) 108: 1303-1314; Parsons, P E et al. Crit. Care Med. (2005) 33: 1-6], protein C [Id., citing Ware, L B, et al. Crit. Care Med. (2007) 35: 1821-28]; and plasminogen activator inhibitor-1(PAI-1; Id., citing Ware, L B, et al. Crit. Care Med. (2007) 35: 1821-28) predict morbidity and mortality in ALI.

Following infection or trauma, upregulation of proinflammatory cytokines occurs as a direct response and/or as a marker of ongoing cellular injury. Baseline and persistently elevated plasma levels of interleukin (IL)-6, IL-8, and tumor necrosis factor (TNF)-α were found to be strongly predicative of mortality [Id., citing Meduri, G U et al. Chest (1995) 108: 1303-14]. A large prospective study involving the ARDS Net trial of lower versus higher tidal volume showed that even after adjustments for ventilator strategy, severity of illness and organ dysfunction, higher plasma levels of IL-6 and IL-8 were independently associated with fewer organ failure- and ventilator-free days, and elevated IL-6 and IL-8 independently predicted higher mortality [Id., citing Parsons, P E, et al. Crit. Care Med. (2005) 33: 1-6; discussion 230-32]. Several studies have demonstrated that lower tidal volume ventilation can attenuate the cytokine responses, potentially reflecting the ability to indirectly modulate the inflammatory response as well as decreasing ventilation-induced lung epithelial injury [Id., citing Parsons, P E et al. Crit. Care Med. (2002) 33: 1-6; Stuber, F. et al. Intensive Care Med. (2002) 28: 834-41; Ranieri, V M, et al. JAMA (1999) 282: 54-61; Levitt, J E et al. J. Intensive Care Med. (2009) 24: 151-67]. Alterations in coagulation and fibrinolysis also occur in lung injury, specifically protein C and plasminogen activator inhibitor-1 [Id., citing Ware, L B et al. Crit. Care Med. (2007) 35: 1821-28]. Compared to controls and patients with acute cardiogenic pulmonary edema, lower plasma levels of protein C and higher plasma levels of plasminogen activator inhibitor-1 were strong independent predictors of mortality, as were ventilator-free and organ-failure-free days.

Microvascular endothelial injury leads to increased capillary permeability. This alteration in permeability permits the efflux of protein-rich fluid into the peribronchovascular interstitium, ultimately crossing the epithelial barrier into the distal airspaces of the lung. [Id., citing Pugin, J. et al. Crit. Care Med. (1999) 27: 304-312]. Several studies have documented increased release of von Willebrand factor (vWf) [Id., citing Ware, L B, et al. Crit. Care Med. (2001) 29: 2325-31; Ware, L B et al. Am. J. Respir. Crit. Care Med. (2004) 170: 766-72; Flori, H R et al. Pediatr. Crit. Care Med. (2007) 8: 96-101] and upregulation of intracellular adhesion molecule-1 (ICAM-1) [Id., citing Flori, H R, et al. Pediatr. Crit. Care Med. (2003) 4: 315-321; McClintock, D. et al. Crit. Care (2008) 12: R41; Calfee, C S et al. Intensive Care Med. (2009) 35: 248-57] following endothelial injury. Both of these biomarkers are independent predictors of mortality.

Transepithelial neutrophil migration is an important feature of acute lung injury, because neutrophils are the primary perpetrators of inflammation. Excessive and/or prolonged activation of neutrophils contributes to basement membrane destruction and increased permeability of the alveolar-capillary barrier. Migrating groups of neutrophils result in the mechanical enlargement of paracellular neutrophil migratory paths [Id., citing Zemans, R L et al. Am. J. Respir. Cell Mol. Biol. (2009) 40: 519-35] Neutrophils also release damaging pro-inflammatory and pro-apoptotic mediators that act on adjacent cells to create ulcerating lesions [Id., citing Zemans, R L et al. Am. J. Respir. Cell Mol. Biol. (2009) 40: 519-35; Downey, G P et al. Chest (1999) 116: 46S-54S] One of the best studied neutrophil mediators, elastase, appears to degrade epithelial junctional proteins, to possess pro-apoptotic properties, and perhaps to have direct cytotoxic effects on the epithelium [Id., citing Ginzberg, H H, et al. Am. J. Physiol. Gastrointest. Liver Physiol. (2001) 281: G705-G717; Ginzberg, H H et al. am. J. Physiol. Gastrointest. Liver Physiol. (2004) 287: G286-G298; Martin, T R et al. Proc. Am. Thorac. Soc. (2005) 2: 214-220; Matute-Bello, G. et al. Infect. Immun. (2001) 69: 5768-76; Matute-Bello, G., Martin, T R. Crit Care (2003) 7: 355-58] In some animal models, neutrophil depletion can be protective [Id., citing Zemans, R L et al. Am. J. Respir. Cell Mol. Biol. (2009) 40: 519-35; Shasby, D M et al J. Appl. Physiol. (1982) 52: 1237-44; Shasby, D M et al. Am. Rev. Respir. Dis. (1982) 125: 443-47; Abraham, E. et al. Am. J. Physiol. Lung Cell Mol. Physiol. (2000) 279: L1137-45]. However, acute lung injury can also develop in the absence of circulating neutrophils, indicating that neutrophil-independent pathways can also cause lung injury [Id., citing Martin, T R, et al. J. Clin. Invest. (1989) 84: 16009-19].

Normally, type I and type II alveolar epithelial cells form tight junctions with each other, selectively regulating the epithelial barrier. Increased permeability of this membrane during the acute phase of lung injury leads to the influx of protein-rich edema fluid into alveolar space. Type I and II epithelial injury leads to disruption of normal fluid transport via downregulated epithelial Na channels and Na+/K+ATPase pumps, impairing the resolution of alveolar flooding [Id., citing Ware, L B, Matthay, M A. N. Eng. J. Med. (2000) 342: 1334-49; Pugin, J. et al. Crit. Care Med. (199) 27: 304-312]. It has been reported that alveolar edema fluid from ALI patients downregulated the expression of ion transport genes responsible for vectorial fluid transport in primary cultures of human alveolar epithelial type II cells [Id., citing Lee, J W, et al. J. Biol. Chem. (2007) 282: 24109-119]. Conversely, gene expression for inflammatory cytokines IL-8, TNF-α, and IL-1β increased by 200, 700, and 900%, respectively. In functional studies, net vectorial fluid transport was also reduced (0.02±0.05 vs. 1.31±0.56 μL/cm²/h, p<0.02). Alveolar epithelial type II cell injury also leads to a loss of surfactant production, [Id., citing Greene, K E, et al. Am. J. Respir. Crit. Care med. (1999) 160: 1843-50] decreasing overall pulmonary compliance. Finally, type II epithelial cells normally drive the epithelial repair process; loss of this function can lead to disorganized, fibrosing repair [Id., citing Bitterman, P B. Am. J. Med. (1992) 92: 39S-343S].

Alveolar epithelial biomarkers, including surfactant D (SP-D) and the receptor for advanced glycation end-products (RAGE), are validated biomarkers for lung epithelial injury. SP-D, secreted by type II epithelial cells, has anti-inflammatory properties and promotes pathogen phagocytosis and neutrophil recruitment. A prospective study from the large ARDS Network low tidal volume ventilation cohort (563 patients) reported that higher baseline plasma SP-D levels were independently associated with mortality and fewer ventilator- and organ-failure free days after controlling for severity of illness, clinical covariates, and ventilator strategy [Id., citing Eisner, M D, t al. Thorax (2003) 58: 983-88]. RAGE, a transmembrane immunoglobulin primarily expressed on type I epithelial cells, is elevated in the plasma and edema fluid of patients with ALI compared to those with hydrostatic edema [Id., citing Uchida, T. et al., Am. J. Respir. Crit. Care Med. (2006) 173: 1008-15]. The ARDS Network plasma samples from the low versus high tidal volume trial were used to further investigate the relationship of RAGE and ALI [Id., citing Calfee, C S, et al. Thorax (2008) 63: 1083-89]. This study reported that higher RAGE levels were associated with increased morbidity and mortality and fewer ventilator-free and organ-failure free days in the higher tidal volume cohort. These findings persisted after adjustment for age, gender, severity of illness, and the presence of sepsis or trauma. RAGE levels declined in both groups; however, there was a 15% greater reduction (p=0.02) in day 3 RAGE levels in the lower tidal volume cohort.

Resolution of ALI/ARDS is primarily dependent on a timely and orderly repair of the alveolar gas exchange apparatus. For gas exchange to improve, alveolar fluid transport must be upregulated, clearing the airspace of protein-rich edema fluid, and restoring the normal secretion of surface active material from alveolar type II cells [Id., citing Matthay, M A, Zimmerman, G A. Am. J. Respir. Cell Mol. Bio. (2005) 33: 319-27; Matthay, M A, et al. Physiol. Rev. (2002) 82: 569-600].

Treatment of acute lung injury is based in both ventilatory and nonventilatory strategies. To date, the most significant advances in the supportive care of lung injury patients have been associated with improved ventilator management. Several clinical trials have shown that a large number of pharmacologic strategies have not been effective in reducing mortality.

The best evidence for the value of a lung protective strategy in patients with ALI is the National Heart, Lung, and Blood Institute (NHLBI) ARDS network's multicenter, randomized controlled trial of 861 patients with ALI/ARDS [Id., citing De Campos, T. N. Engl. J. Med. (2000) 342: 1301-08] In this study, patients were randomized to 6 mL/kg tidal volume versus 12 mL/kg tidal volume with plateau pressure restrictions (<30 vs. <50 cm H2O). Mortality in the low tidal volume group was significantly lower than the high tidal volume group (31 vs. 40%, p=0.007). Patients ventilated with low tidal volume also had more ventilator free and nonpulmonary organ failure-free days. Clinical risk factors including sepsis, aspiration, pneumonia, and trauma did not affect the efficacy of the low tidal volume strategy [Id., citing Eisner, M D, et al. Am. J. REspir. Crit. Care Med. (2001) 164: 231-36] This strategy even attenuated the inflammatory response (IL-6 and IL-8) associated with acute lung injury [Id., citing Parsons, P E et al. Crit. Care Med. (2005) 33: 1-6, discussion 230-32].

Optimal fluid management has been a controversial topic. In 2006, the NHLBI ARDS Network published the findings of their prospective, randomized controlled trial of fluid conservative versus fluid liberal management strategy [Id., citing Wiedemann, J P., et al. N. Engl. J. Med. (2006) 354: 2564-75]. Although there was not a significant difference in mortality, the fluid conservative strategy improved oxygenation and severity of lung injury as well as reduced the duration of mechanical ventilation. The incidence of nonpulmonary organ failure, specifically renal failure, and shock, did not increase.

Numerous potential pharmacologic treatments have been investigated. Despite earlier encouraging preclinical evidence, phase III trials have not supported the use of exogenous surfactant, inhaled nitric oxide, intravenous prostaglandin El, glucocorticoids, Ketoconazole, Lisofylline, N-acetylcysteine, or activated protein C as treatments for ALI.

Viral Mediated ALI

The underlying pathophysiology of virally mediated ALI is not well understood, and it is likely that there are unique signature mechanisms to each viral strain that converge onto a common end pathway resulting in diffuse alveolar damage (DAD). It remains to be seen whether epithelial injury is the primary lesion or is coincident to endothelial injury. Most community-acquired respiratory viral pneumonias are inhaled and bind to receptors in the upper respiratory tract. Although the viruses initially infect the respiratory epithelium, it is possible that this is merely a portal of entry, and the important steps in alveolar damage are mediated primarily by endothelial injury resulting in elaboration of cytokines and chemokines and recruitment of both innate and adaptive immune cells. The specific cytokine profiles vary by viral pathogen, which may be driven by macrophages, epithelial cells, endothelial cells, or some combination of crosstalk.

If lung injury is not primarily mediated by viral infection, but rather is a result of the inflammatory host response, then viral clearance may not be central to the resolution of lung injury [Hendrickson, C M, Matthay, M A Semin. Respir. Crit. Care Med. (2013) 34: 475-86].

ARDS

Increasingly, ARDS is recognized as a heterogeneous syndrome that is under-recognized and undertreated. It develops most commonly in the setting of bacterial and viral pneumonia, nonpulmonary sepsis (with sources that include the peritoneum, urinary tract, soft tissue and skin), aspiration of gastric and/or oral and esophageal contents (which may be complicated by subsequent infection), and major trauma (such as blunt or penetrating injuries or burns). Several other less common scenarios associated with the development of ARDS include acute pancreatitis, transfusion-associated acute lung injury (TALI); drug overdose; near drowning; hemorrhagic shock or reperfusion injury (including after cardiopulmonary bypass and lung resection), and smoke inhalation (often associated with cutaneous burn injuries. [Matthay, M A, et al. Nature Revs. (2019) 5: 18].

ARDS is defined by acute hypoxemia (the ratio of partial pressure of arterial oxygen (PaO₂) to the fraction of inspired oxygen (FiO₂)≤200 mmHg on positive end-expiratory pressure (PEEP) ≥5 cm H₂O) with bilateral infiltration on chest imaging which cannot be fully explained by cardiac failure or fluid overload [Id., citing Han, S. Mallampalli, R K. J. Immunol. (2015) 194 (3): 855-60, citing Force, A D T, et al. JAMA (2012) 307: 2526-33]. It is a form of severe hypoxemic respiratory failure characterized by inflammatory injury to the alveolar capillary barrier with extravasation of protein-rich edema fluid into the airspace.

In ARDS, there is increased permeability to liquid and protein across the lung endothelium, which then leads to edema in the lung interstitium. Next, the edematous fluid translocates to the alveoli, often facilitated by injury to the normally tight barrier properties of the alveolar epithelium. Increased alveolar-capillary permeability to fluid, proteins, neutrophils and red blood cells (resulting in their accumulation into the alveolar space) is the hallmark of ARDS [Id., citing Matthay, M A, et al. J. Clin. Invest. (2012) 122: 2731-40; Bachofen, M., Weibel, E R. Clin. Chest Med. (1982) 3: 35-56; Fein, A. et al. Am. J. Med. (1979) 67: 32-38].

Arterial hypoxemia in patients with ARDS is caused by ventilation-to-perfusion mismatch as well as right-to-left intrapulmonary shunting. In addition, impaired excretion of carbon dioxide is a major component of respiratory failure, resulting in elevated minute ventilation that is associated with an increase in pulmonary dead space (that is, the volume of a breath that does not participate in carbon dioxide excretion). Elevation of pulmonary dead space and a decrease in respiratory compliance are independent predictors of mortality in ARDS [Nuckton, T J et al. N. Engl. J. Med. (2002) 346: 1281-86].

Diffuse alveolar damage (DAD) is the classic histopathological hallmark of ARDS. Interstitial and alveolar edema are key features of DAD in the acute ‘exudative’ phase (˜7 days). Eosinophilic depositions termed hyaline membranes are also defining features of DAD [Id., citing Katzenstein, A L et al. Am. J. Pathol. (1976) 85: 209-28; Mendez, J L & Hubmayr, R D. Curr. Opin. Crit. Care (2005) 11: 29-36; Cardinal-Fernandez, P. et al. Ann. Am. Thorac Soc. (2017) 14: 844-50]. The other findings include alveolar hemorrhage, accumulation of white blood cells (usually predominantly neutrophils), fibrin deposition and some areas of alveolar atelectasis (collapse). After the initial exudative phase, ATII cell hyperplasia follows in a ‘proliferative’ phase that can last >3 weeks in survivors; interstitial fibrosis can also occur in this phase.

DAD is present in only a subset of patients with clinical ARDS, and pathological heterogeneity is evident [Id., citing Mendez, J L & Hubmayr, R D. Curr. Opin. Crit. Care (2005) 11: 29-36; Cardinal-Fernandez, P. et al. Ann. Am. Thorac. Soc. (2017) 14: 844-50; Thille, A W et al. Lancet Respir. Med. (2013) 1: 395-401; Thille, A W et al. Am J. Respir. Crit. Care Med. (2013) 187: 761-7]. For example, one study carried out over two decades (1991-2010) on post-mortem samples reported that 45% of patients who met the Berlin criteria for ARDS had DAD, whereas the other 55% had alveolar inflammation consistent with acute pneumonia with infiltration of neutrophils in the alveoli and distal bronchioles [Id., citing Thille, A W et al. Am J. Respir. Crit. Care Med. (2013) 187: 761-7]. This study also found that the incidence of DAD declined in the decade after lung-protective ventilation was implemented. Recent reports also indicate key temporal features of histological progression, identify the association of DAD with severity of ARDS and provide evidence that the first 7 days after onset represent a critical window for potential therapeutic intervention [Id., citing Thille, A W et al. Lancet Respir. Med. (2013) 1: 395-401; Thille, A W et al. Am J. Respir. Crit. Care Med. (2013) 187: 761-7]. In addition, one meta-analysis of open lung biopsy samples in patients with ARDS found that DAD was present in only 48% of the patients and was associated with a higher mortality [Id., citing Cardinal-Fernandez, P. et al. Chest (2016) 149: 1155-64]. Neither the severity of hypoxemia nor the sequential organ failure assessment score were different in patients with or without DAD on lung biopsy.

Alterations in endothelial and epithelial cells are critical features of acute alveolar injury in ARDS [Id., citing Bachofen, M., Weibel, E R. Clin. Chest Med. (1982) 3: 35-56; Bachofen, M. & Weibel, E R. Am. Rev. Respir. Dis. (1977) 116: 589-615]. For example, early involvement of ATI cells is frequently dramatic and includes focal epithelial destruction and denudation of the alveolar basement membrane [Id., citing Katzenstein, A L et al. Am. J. Pathol. (1976) 85: 209-228; Bachofen, M. & Weibel, E R. Am. Rev. Respir. Dis. (1977); Bachofen, M. & Weibel, E R. Am. Rev. Respir. Dis. (1977) 116: 589-615]. By contrast, alveolar endothelial cells are usually morphologically preserved and the endothelial lining is continuous, demonstrating that even ultrastructural analyses cannot precisely detect abnormalities in the normal barrier properties that regulate fluid and protein flux across the lung capillaries [Id., citing Bachofen, M. & Weibel, E R. Clin. Chest Med. (1982) 3: 35-56]. Epithelial cell necrosis is usually described in the exudative phase [Id., citing Cardinal-Fernandez, P. et al. Ann. Am. Thoracic soc. (2017) 14: 845-50; Tomashefski, J. F. Jr. Clin. Chest Med. (2000) 435-66], although evidence for apoptosis has also been reported [Id., citing Albertine, K H, et al. Am. J. Pathol. (2001) 161: 1783-96; Bastarache, J A et al. Am. J. Physiol. Lung Cell Mol. Physiol. (2009) 297: L1035-L10411. Early epithelial injury is followed rapidly by ATII cell proliferation [Id., citing Bachofen, M. & Weibel, E R. Clin. Chest Med. (19982) 3: 35-56; Katzenstein, A L et al. Am. J. Pathol. (1976) 85: 209-28; Thille, A W et al. Lancet Respir. Med. (2013) 1: 395-401; Tomashefski, J. F. Jr. Clin. Chest Med. (2000) 435-66]. Injured but intact alveolar epithelial cells seem to drive release of pro-coagulant factors and intra-alveolar fibrin deposition [Id., citing Wang, L, et al. Am. J. REspir. Cell Mol. Biol. (2007) 36: 497-503; Bastarache, J A, e al. Am. J. Physiol. Lung Cell Mol. Physiol. (2009) 297: L1035-41], which is also deposited adjacent to endothelial cells in injured alveoli [Id., citing Bachofen, M. & Weibel, E R. Clin. Chest Med. (1982) 3: 35-56; Katzenstein, A L et al. Am. J. Pathol. (1976) 85: 209-28; Tomashefski, J. F. Jr. Clin. Chest Med. (2000) 435-66].

Endothelial Damage

Although the nature of endothelial cell alteration in clinical ARDS is incompletely understood, endothelial damage and injury are commonly described, and recent evidence suggests that apoptosis [Id., citing Matthay, M A et al., J. Clin. Invest. (2012) 122: 2731-40] and alternative cell death pathways, such as pyroptosis [Id., citing Cheng, K T et al. J. Clin. Invest. (2017) 127: 4124-35] might be involved. Conceptually, an increase in lung vascular permeability can occur because of a functional breakdown in endothelial junctions or by death of endothelial cells. Ultrastructural alterations of alveolar endothelial cells are frequently subtle compared with the dramatic epithelial cell disruption observed in autopsy analysis [Id., citing Bachofen, M. & Weibel, E R. Clin. Chest Med. (1982) 3: 35-56], suggesting functional barrier impairment. Experimental evidence has shown that endothelial cell activation can occur, induced by inflammatory signals from microorganisms (including lipopolysaccharide and other toxins) and lung white blood cells in response to pathogens (as in pneumonia or nonpulmonary sepsis), injury from aspiration syndromes, ischemia-reperfusion (as in trauma-induced shock) or blood product transfusions as in transfusion-related acute lung injury (TRALI) [Id., citing Matthay, M A, et al. J. Clin. Invest. (2012) 122: 2731-40]. Endothelial cell activation may result in mediator generation (such as angiopoietin 2) and leukocyte accumulation (accompanied by upregulation of P-selectin and E-selectin (cell adhesion molecules) in the lung microvessels, especially in the post-capillary venules [Id., citing Matthay, M A, et al. J. Clin. Invest. (2012) 122: 2731-40].

Platelet and neutrophil deposition characteristically occur, often as neutrophil-platelet aggregates, as a result of endothelial cell activation. Neutrophils and platelets seem to play a synergistic role in causing an increase in lung vascular permeability to protein. Endothelial disruption can also be caused by pathogens and their toxins; endogenous danger-associated molecular patterns; barrier-destabilizing factors generated by alveolar macrophages, circulating leukocytes and platelets; and pro-inflammatory signaling molecules such as tumor necrosis factor (TNF), the inflammasome product IL-1β, angiopoietin 2, vascular endothelial growth factor, platelet-activating factor and others [Id., citing Matthay, M A, et al. J. Clin. Invest. (2012) 122: 2731-40]. Increased systemic vascular permeability frequently also occurs, often contributing to hypovolemia and multiple organ failure.

Mechanistic examination of disrupted endothelial barriers has required experimental models. A large-animal (sheep) preparation demonstrated that clinically relevant insults, including intravenous bacteria, lipopolysaccharide and microemboli, cause an increase in lung endothelial permeability and filtration, and that there are different responses to these insults by the endothelial and epithelial barriers [Id., citing Brigham, K L, et al. J. Clin. Invest. (1974) 54: 792-804; Wiener-Kronish, J P, et al. J. Clin. Invest. (1991) 88: 864-75]. Although the duration of increased lung endothelial permeability induced by specific insults in clinical ARDS is unknown, this model and more recent studies in mice suggest that it can persist for many hours to weeks [Id., citing Brigham, K L, et al. J. Clin. Invest. (1974) 54: 792-804; Wiener-Kronish, J P, et al. J. Clin. Invest. (1991) 88: 864-75; Gotts J E, et al. Am. J. Physiol. Lung Cell Mol. Physiol. (2014) 307: L395-L406]. In experimental models of influenza pneumonia, for example, the persistent duration of increased lung vascular permeability is associated with lung injury and slow recovery [Id., citing Gotts J E, et al. Am. J. Physiol. Lung Cell Mol. Physiol. (2014) 307: L395-L406].

VE-Cadherin Disruption

Studies using cultured endothelium and murine models indicate that hemophilic calcium-dependent vascular endothelial cadherin (VE-cadherin) bonds between adjacent endothelial cells are critical for basal lung microvascular integrity, and that their ‘loosening’ is central in increased alveolar-capillary permeability in inflammatory acute lung injury [Id., citing Matthay, M A, et al. J. Clin. Invest. (2012) 122: 2731-40]. VE-cadherin and TIE2, an endothelial receptor kinase, act in concert to establish junctional integrity and are regulated by vascular endothelial-protein tyrosine phosphatase (VE-PTP; also known as receptor-type tyrosine-protein phosphatase 13). Genetic or pharmacological manipulation of the molecular interactions and activities of VE-cadherin, TIE2 and VE-PTP alters alveolar leak in a complex fashion in mice [Id., citing Matthay, M A, et al. J. Clin. Invest. (2012) 122: 2731-40, Frye, M. et al. J. Exp. Med. (2015) 212: 2267-87]. VE-cadherin function and adherens junction stability are also regulated by cytoskeletal interactions, small GTPases and other intracellular modulators, multiple molecular interactions (including associations with catenins, plakoglobin and VE-PTP) and phosphorylation and dephosphorylation events [Id., citing Matthay, M A, et al. J. Clin. Invest. (2012) 122: 2731-40, Giannotta, M. et al. Dev. Cell (2013) 26: 441-54]. Destabilizing signals from pathogens or inflammatory cells and mediators responding to infectious agents induce phosphorylation of VE-cadherin and its internalization, frequently by altering activity and balance of GTPases [Id., citing Giannotta, M. et al. Dev. Cell (2013) 26: 441-54]. Dissociation of VE-PTP from VE-cadherin is required for loosening of endothelial cell junctions and inflammatory alveolar protein leak in mice [Id., citing Broermann, A. et al. J. Exp. Med. (2011) 208: 2393-2401].

Recent observations indicate that inflammation-induced weakening of endothelial junctions is a process involving at least two steps, including modification of VE-cadherin contacts and alterations in the endothelial actomyosin system [Id., citing Frye, M. et al. J. Exp. Med. (2015) 212: 2267-87]. Genetic or pharmacological manipulation of VE-PTP can alter alveolar endothelial junctions via TIE2-dependent influences on the cytoskeleton independently of VE-cadherin [Id., citing Frye, M. et al. J. Exp. Med. (2015) 212: 2267-87]. Although parallel experiments with cultured human endothelial cells suggest translational relevance [Id., citing Frye, M. et al. J. Exp. Med. (2015) 212: 2267-87], direct recapitulation of these observations to alveolar endothelial barrier disruption in patients with ARDS has not been established. Nevertheless, administration to mice of an antibody against VE-cadherin resulted in intravascular sequestration of neutrophils and platelets, alveolar neutrophil accumulation and pulmonary edema [Id., citing Corada, M. et al. Proc. Nat. Acad. Sci. USA (1999) 96: 9815-20], mimicking the histological pattern in clinical ARDS [Id., citing Bachofen, M. & Weibel, E R. Clin. Chest Med. (1982) 3: 35-56]. Substantial alveolar edema in experimental acute lung injury was not accompanied by widespread overt disruption of endothelial cell junctions detectable by electron microscopy [Id., citing Frye, M. et al. J. Exp. Med. (2015) 212: 2267-87], consistent with ultrastructural observations of lung tissue from patients with ARDS in which the endothelium was found to be largely continuous and endothelial cell junctions were, for the most part, morphologically intact [Id., citing Bachofen, M. & Weibel, E R. Clin. Chest Med. (1982) 3: 35-56]. Thus, in animal models and human ARDS, changes in paracellular permeability to protein seem to occur in the absence of dramatic alterations in the morphology of the lung endothelium.

Immune Cell Recruitment to the Lung

Re-establishing endothelial junctional bonds may mitigate both endothelial leak and excessive myeloid leukocyte accumulation in ARDS [Id., citing Matthay, M A, et al. J. Clin. Invest. (2012) 122: 2731-40]. Indeed, genetic replacement of VE-cadherin with a fusion construct that prevented its internalization in response to inflammatory signals greatly reduced alveolar neutrophil accumulation in lipopolysaccharide-challenged mice and reduced vascular permeability [Id., citing Schulte, D. et al. EMBO J. (2011) 30: 4157-70]. Analysis of samples from patients and lipopolysaccharide-challenged volunteers indicated that synergistic activity of chemokines contributes to neutrophil recruitment [Id., citing Williams, A E et al. Thorax (2017) 72: 66-73]. Other signaling molecules are also likely to be involved [Id., citing Matthay, M A, et al. J. Clin. Invest. (2012) 122: 2731-40]. Degranulation of neutrophils with release of intracellular enzymes such as neutrophil elastase and oxidant products contributes to the lung injury [Id., citing Matthay, M A, et al. J. Clin. Invest. (2012) 122: 2731-40].

Neutrophils in the intravascular and extravascular compartments in acute lung injury are often associated with platelets, which have intricate thrombo-inflammatory activities including the ability to trigger deployment of neutrophil extracellular traps (NETs) [Id., citing Matthay, M A, et al. J. Clin. Invest. (2012) 122: 2731-40]. NETS are a meshwork of nuclear chromatin that is released into the extracellular space by neutrophils undergoing apoptosis at sites of infection, and serve as a scaffold that traps extracellular microbes to enhance their phagocytosis by other phagocytes. They correlate with alveolar-capillary and epithelial barrier disruption in ARDS and experimental models [Id., citing Lefrancais, E. et al. JCI Insight (2018) 3: 98178]. Intra-alveolar macrophages play an important part in releasing chemotactic factors such as IL-8 and chemokines such as CC-chemokine ligand 2 (also known as MCP1) that enhance the recruitment of neutrophils and monocytes into the lung, particularly in response to acute pulmonary infections.

Epithelial Injury and Repair

In the early phase of experimental acute lung injury, the epithelium is more resistant to injury than the endothelium [Id., citing Wiener-Kronish, J P et al. J. Clin. Invest. (1991) 88: 864-75], but some degree of epithelial injury is characteristic of ARDS. The extent of epithelial injury is also an important determinant of the severity of ARDS. The epithelium can be injured directly, for example, by bacterial products, viruses, acid, oxygen toxicity (hyperoxia), hypoxia and mechanical forces, or by inflammatory cells or their products, as in sepsis, transfusion-related acute lung injury and pancreatitis.

As with endothelial injury [Id., citing Matthay, M A, et al. J. Clin. Invest. (2012) 122: 2731-40; 57-59], epithelial injury includes dissociation of intercellular junctions [Id., citing Short, K R et al., Eur. Respir. J. (2016) 47: 954-66; Schlingmann, B. e t al. Nat. Commun. (2016) 7: 12276]. Release of cell-free hemoglobin from red blood cells contributes to paracellular permeability by oxidant-dependent mechanisms. On the basis of experimental studies, the cyclo-oxygenase inhibitor acetaminophen reduces the tyrosine radical that results from oxidation of cell-free hemoglobin (Fe⁴⁺ oxidation state to Fe³⁺ oxidation state), thereby reducing the potential for lipid peroxidation [Id., citing Shaver, C M et al. JCI Insight (2018) 3: 98546]. In addition, apoptotic or necrotic epithelial cell death [Bachofen, M. & Weibel, E R. Clin. Chest Med. (1982) 3: 35-56; Albertine, K H, et al. Am. J. Pathol. (2002) 161: 1783-96; Budinger, G R et al. am. J. Respir. Crit. Care Med. (2011) 183: 1043-54; Hogner, K. et al. PLoS Pathog. (2013) e1003188] is a key feature of alveolar injury in ARDS and can be directly caused by lytic viral infections, bacterial toxins, acid, hypoxia, hyperoxia and mechanical stretch [Id., citing Vaughan, A E et al. Nature (2015) 517: 621-25; Imai, Y. et al. JAMA (2003) 289: 2104-12]. Neutrophil-derived mediators also induce epithelial cell death via multiple mechanisms, including oxidation of soluble TNF ligand superfamily member 6 (FasL) [Id., citing Herrero, R. et al., J. Clin. Invest. (2011) 121: 1174-90]0 and NETs [Id., citing Saffarzadeh, M. et al. PLoS One (2012) 7: e32366], whereas inflammatory macrophages can induce cell death via mechanisms including secretion of TNF-related apoptosis-inducing ligand (TRAIL) [Id., citing Hogner, K. et al. PLoS Pathog. (2013) 9: e1003188]. During infection, endogenous mechanisms (such as syndecan-1-dependent MET-AKT signaling) can limit cell death. [Id., citing Brauer, R. et al. Am. J. Respir. Crit. Care Med. (2016) 194: 333-44].

Additionally, plasma membrane wounding without cell death (that is, sublethal injury) may result from bacterial pore-forming toxins and/or overdistention from positive-pressure ventilation with high tidal volumes. After membrane wounding by Staphylococcus aureus toxin, it was reported that calcium waves spread through gap junctions to neighboring epithelial cells, inducing widespread mitochondrial dysfunction and loss of barrier integrity without cell death. [Id, citing Hook, J L et al. J. Clin. Invest. (2018) 128: 1074-86]. Mitochondrial dysfunction is common in lung injury and may be induced by various mechanisms, including elevated CO₂ concentrations (hypercapnia) [Id., citing Vohwinkel, C U et al. J. Biol. Chem. (2011) 286: 37067].

Repair of the injured epithelium is critical for clinical recovery [Id., citing Ware, L B, Matthayy M A. Am. J. Respir. Crit. Care Med. (2001) 163: 1376-83]. The time frame for epithelial repair may be 2-3 days or several weeks. Because ATI cells provide >95% of the normal surface area of the alveolar epithelium and facilitate gas exchange, the process of generating new ATI cells is critical to the complete repair process. However, initially the proliferation of ATII cells can provide a provisional epithelial barrier before they transdifferentiate into ATI cells. Many growth factors contribute to ATII cell proliferation; although ATII cells are the default progenitors responsible for creating new alveolar epithelial cells through proliferation, in severe injury, alternate progenitor cells may be mobilized. These alternate progenitor cells include club cells (meaning secretory cells that normally line the airways) [Id., citing Hogan, B L et al. Cell Stem Cell (2014) 15: 123-38], bronchoalveolar stem cells, and keratin-5-expressing (KRTS+) cells [Id., citing Vaughan, A E et al. Nature (2015) 517: 621-25; Ray, S., et al. Stem Cell Rep. (2016) 7: 817-25]. Expansion of KRTS+epithelial progenitors is driven by HIF-NOTCH and fibrocyte growth factor receptor 2 signaling [Id., citing Quantius, J. et al. PLoS Pathog. (2016) 12: e1005544], with ATII cell fate induced by WNT-β-catenin and impeded by NOTCH and HIF [Id., citing Xi, Y. et al. Nat. Cell Biol. (2017) 19: 904-14]. The mechanisms underlying ATII-to-ATI transdifferentiation are less well understood; studies have suggested that deactivation of WNT-β-catenin is necessary [Id., citing Nabban, A N et al. Science (2018) 359: 1118-23]. Mouse models of lung injury have informed our knowledge of the regenerative role of the alternative progenitors, although there is evidence that some of these progenitors exist in humans as well [Id., citing Xi, et al. Nat. Cell Biol. (2017) 19: 904-14].

Repair of the alveolar epithelium in vivo is regulated by crosstalk between multiple alveolar cell types and the extracellular matrix. Although injury-inducing, immune cells and their mediators may also promote epithelial repair [Id., citing Dial, C F et al, Am. J. Respir. Cell Mol. Biol. (2017) 57: 162-73; Zemans, R L et al. Proc. Nat. Acad. Sci. USA (2011) 108: 15990-95]. Fibroblasts secrete epithelial growth factors and deposit collagen, which, if excessive, can lead to fibrosis. Sublethal epithelial cell injury can also be repaired. For example, plasma membrane pores can be excised by endocytosis or exocytosis and patched by fusion with lipid endomembrane vesicles [Id., citing Cong, X. et al. Am. J. Physiol. Lung Cell Mol. Physiol. (2017) 312: L371-L391]. Additionally, damaged mitochondria are degraded via mitophagy and replaced via biogenesis or mitochondrial transfer [Id., citing Schumacker, P T et al. am J. Physiol. Lung Cell Mol. Physiol. (2014) 306: L962-L1974]. Finally, reassembly of intercellular junctions is regulated by multiple mechanisms, including beneficial effects from angiopoietin 1 [Id., citing Fang, X. et al. J. Biol. Chem. (2010) 285: 26211-11] and signals from the basement membrane [Id., citing Koval, M. et al. Am. J. Respir. Cell Mol. Biol. (2010) 421: 172-80]. The timing of endothelial and epithelial repair in various causes of acute lung injury has not been systematically worked out. Once epithelial barrier integrity is restored, edematous fluid can be reabsorbed to the interstitium either by paracellular pathways or by diffusion through water channels driven by an osmotic gradient that is established by active apical sodium uptake, in part by the epithelial sodium channels and sodium transport through the Na+/K+-ATPase pumps.

Many endogenous reparative mechanisms are specifically inhibited during ARDS. For example, influenza virus infects KRT5+ progenitors [Id., citing Quantius, J. et al. PLoS Pathog. (2016) 12: e1005544]. Influenza infection, hypoxemia, hypercapnia and other factors downregulate sodium channel and/or Na+/K+-ATPase function, resulting in impaired alveolar fluid clearance in patients with ARDS [Id., citing Matthay, M A. Am. J. Respir. Crit. Care Med. (2014) 189: 1301-8; Ware, L B, Matthay, M A. Am. J. Respir. Critic. Care Med. (2001) 163: 1376-83; Gwozdzinska, P. et al. Front. Immunol. (2017) 8: 591; Vadasz, I. et al. Front. Immunol. (2017) 8: 757]. Elevated CO₂ impairs alveolar epithelial cell proliferation [Id., citing Vohwinkel, C U, et al. J. Biol. Chem. (2011) 286: 37067]. Keratinocyte growth factor, while stimulating proliferation, increases the susceptibility of ATII cells to influenza virus infection and mortality in mice [Id., citing Nikolaidis, N M, et al. Proc. Nat. Acad. Sci. USA (2017) 114: E6613-22]. In addition, the many biological changes resulting from both endothelial and epithelial injury, and culminating in protein-rich edematous fluid, contribute to surfactant dysfunction [Id., citing Albert, R K. Am. J. Respir. Crit. Care Med. (2012) 185: 702-8]. Surfactant dysfunction can then increase atelectasis (the collapse or closure of a lung), which in turn can increase the risk of biophysical injury.

Mechanical Ventilator-Associated Lung Injury (VALI)

All the mechanisms that injure the lung endothelium and epithelium lead to pulmonary edema with acute respiratory failure owing to reduced oxygenation, impaired carbon dioxide excretion and decreased lung compliance. The use of mechanical ventilation with supplemental oxygen and positive end-expiratory pressure (PEEP) was life-saving in this context. For many years, the standard therapy with mechanical ventilation support included high tidal volumes (12-15 ml per kg PBW). Nevertheless, a potential contribution of high tidal volumes and elevated airway pressures to worsening acute lung injury was suggested by preclinical studies beginning in 1974 [Id., citing Webb, H H, Tierney, D F. Am. Rev. Respir. Dis. (1974) 110: 556-65; Parker, J C, et al. J. Appl. Physiol. Respir. Environ. Exerc. Pysiol. (1984) 57: 1809-16]. In the ARMA trial in 2000, lower tidal volume and limited airway pressure markedly reduced mortality in patients with ARDS [Id., citing; Brower, R G et al. N. Engl. J. Med. (2000) 342: 1301-8]. The mechanisms for VALI have been established in both experimental and clinical studies. High tidal volume and elevated airway pressure induce biomechanical inflammatory injury and necrosis of the lung endothelium and alveolar epithelium that are associated with release of neutrophil products, including proteases, oxidants and pro-inflammatory cytokines, and a reduction in the capacity of the alveolar epithelium to remove edematous fluid [Id., citing Matthay, M A e al. J. Clin. Invest. (2012) 122: 2731-40; Tremblay, L. et al. J. Clin. Invest. (1997) 99: 944-52; Frank, J A et al. Am. J. Respir. Crit. Care Med. (2002) 165: 242-49]. Clinical studies focused on biology and clinical factors have also confirmed the injurious effects of high tidal volume in patients with ARDS.

Long-Term Mortality

Patients who survive ARDS remain at risk for mortality and may have persistent morbidity. [Chiumello, D. et al. Respiratory Care (2016) 61(5): 689-99]. The severity of the initial acute lung injury/ARDS and the rapidity of its resolution seem to correlate significantly with long-term (1-y) physical function, although the inability to exercise in terms of muscle wasting and weakness has a multifactorial etiology and can be due to extrapulmonary disease. Similarly, ARDS subjects treated with ECMO suffered a loss of health related quality of life (HRQOL) because of pulmonary sequelae at 1 year after ECMO [Id., citing Linden, V B e al. Acta Anaesthesiol. Scand (2009) 53 (94): 489-95], although most of the decline in functional status was attributable to preexisting comorbidities.

Follow-up CT scans of ARDS subjects after ARDS resolution revealed four CT abnormalities: based on the Fleischner Society Glossary: (1) ground glass opacity (defined by a hazy increase in lung attenuation with preservation of bronchial and vascular margins); (2) consolidation or intense parenchymal opacification in the previously published glossary of the society (defined by a homogeneous increase in pulmonary parenchyma attenuation that obscures the margins of vessels and the airway wall); (3) reticular pattern (defined by a collection of innumerable small linear opacities, constituted by interlobular septal thickening, intralobular lines, or the cyst walls of honeycombing); and (4) decreased attenuation (which includes emphysema and small airways disease) [Id., citing Hansell, D M et al. Radiology (2008) 246 (3): 697-722]. In the acute phase of ARDS, the classical morphological CT description is the result of a combination of alveolar flooding (edema), interstitial inflammation, and compression atelectasis, which are associated with overall disease severity and mortality [Id., citing Cressoni, M. et al. Am J. Respir. Crit. Care Med. (2014) 189 (2): 149-58]. Reticular pattern was related to the duration of mechanical ventilation: the more time spent receiving mechanical ventilation, the more reticular pattern in the late phase. These observations were confirmed in 2001, when Nobauer-Huhmann et al. [Id., citing Nobauer-Huhmann, I M et al. Eur. Radiol. (2001) 11 (12): 2436-43] performed a high-resolution CT in a group of survivors at 6-10 months after ARDS due to polytrauma. Pulmonary fibrosis was identified in 87% of 15 subjects. Parenchymal changes, such as thickened interlobular septa, non-septal lines, parenchymal bands, and cystis, were more frequent and pronounced in the non-dependent lung regions compared with the dependent lung regions, and the most severe type of alterations, such as honeycombing and subpleural cystis, were found exclusively in the non-dependent regions. Also, in this study, a clear relationship between extent of lung alteration at follow-up CT scan and the duration of high pressure ventilation (peak pressure >30 cm H₂O) was found. A 2004 study confirmed that pulmonary ARDS may be more vulnerable to ventilator-induced lung injury, leading to more severe sequelae after long-term recovery. [Id., citing Kim, S J et al. Intensive Care Med. (2004) 30 (10): 1960-63]. In patients treated with ECMO support, the most common residual pathological finding in CT scan at 26 (interquartile range 12-50) months was the reticular pattern (76% of subjects), whereas ground glass opacity was found in 24% of subjects 1 year after ARDS [Id., citing Linden, V B et al. Acta Anaesthesiol. Scand. (2009) 53 (4):489-95]. The duration of ECMO treatment was related to the extent of fibrosis. [Id., citing Linden, V B et al. Acta Anaesthesiol. Scand. (200(53 (4):489-95].

The outcome of pulmonary function has been evaluated in various ways, for instance by spirometry, plethysmography, diffusing capacity of the lung for carbon monoxide, maximal oxygen consumption, blood gas analysis at rest and during maximal exercise, and 6-min walk test [Id., citing Pellegrino, R. et al. Eur. Repir. J. (2005) 26 (5): 948-68]. Even if spirometry (to assess static and dynamic lung volumes) indicates a good recovery in terms of lung volumes within 6 months after ARDS, diffusing capacity (in order to assess the capacity of gas exchange across the alveolar barrier) and 6-min walk test (a standardized method to globally evaluate cardiopulmonary function) highlighted a reduction of function that persisted up to 5 years after ARDS [Id.].

Viral-Mediated ARDS

Loss of the endothelial barrier through both indirect (paracrine) and direct (cytopathic) mechanisms has been hypothesized to contribute to ARDS pathology resulting from severe influenza.

Influenza virus infects the bronchial epithelium, leading to epithelial injury, apoptosis and frank desquamation [Id., citing Kuiken, T. and Taubenberger, J K Vaccine (2008) 26 (Suppl. 4): D59-D66]. In uncomplicated infections, these changes to the airway epithelium are transient and the process of repair is evident within days. However, in primary viral pneumonia, influenza virus spreads from the upper to the lower respiratory tract [Id., citing Mauad, T. et al. Am. J. Respir. Crit. Care Med. (2010) 181: 72-79], infecting the distal lung, particularly type 1 pneumocytes and ciliated bronchiolar epithelium. This leads to damage to the alveoli including frank alveolar denudement [Id., citing Kuiken, T. and Taubenberger, J K Vaccine (2008) 26 (Suppl. 4): D59-D66]. Type II pneumocytes and alveolar macrophages also can be infected.

Lung epithelial apoptosis alone is not sufficient to induce leak of the lung alveolocapillary membrane [Id., citing Mura, M. et al. Am. J. Pathol. (2010) 176: 1725-34]. Influenza virus is known to interfere with alveolar fluid clearance by interfering with the function of the epithelial sodium channel (ENaC), which regulates fluid absorption from the alveolar space [Id., citing Chen, X J et al. Am. J. Physiol. Lung Cell Mol. Physiol. (2004) 287: L366-73: Kunzelmann, K. et al. Proc. Nat. Acad. Sci. USA (2000) 97: 10282-87; Lazrak, A. et al. FASEB J. (2009) 23: 3829-42; Wolk, K E et al. (2008) Am. J. Respir. Crit. Care Med. 178: 969-76]. A decrease in alveolar fluid clearance correlates with poor outcomes in patients with ARDS [Id., citing Ware, L B & Matthay, M A Am. J. Respir. Crit. Care Med. (2001) 163: 1376-83]. These effects would diminish or delay the resolution of pulmonary edema, and may also contribute to its formation [Id., citing Lee, J W et al. J. Biol. Chem. (2007) 282: 24109-119].

A loss of lung endothelial barrier function is a major determinant of the formation of pulmonary edema in ARDS [Id., citing Maniatis, N A and Orfanos, S E Curr. Opin. Crit. Care (2008) 14: 22-30]. Because the average thickness of the alveolocapillary membrane is just over 1 μM, including the alveolar epithelium, the thin interstitium and the lung microvascular endothelium [Weibel, E R, Knight, B W J. Cell Biol. (1964) 21: 367-96], and in some regions, the barrier is as thin as 100-200 nm, an interaction between the virus and the endothelium is plausible. Indeed, infection of the alveolar epithelium leads to cell death and the release of new influenza virions [Id., citing Kuiken, T. and Taubenberger, J K Vaccine (2008) 26 (Sunni. 4): D59-D66; Mori, I et al. J. Gen Virol. (1995)] 76 (Pt 11): 2869-73], exposing the endothelium to viral particles and to epithelial and leukocyte paracrine factors.

It has been hypothesized that there are multiple overlapping mechanisms by which influenza virus could induce increased lung endothelial permeability. One of the main contributors is pro-inflammatory cytokines produced by leukocytes, lung epithelium and the lung endothelium. For example, infections with both H5N1 avian influenza virus [Id., citing Schmolke, M. et al. J. Immunol. (2009) 183: 5180-89] and influenza A (H1N1pdm09) virus [Id., citing Bermejo-Martin, J F et al. Crit. Care (2009) 13: 82201] have been associated with markedly elevated circulating cytokines, including TNFα and IL-6, which are well known to cause increased endothelial permeability [Id., citing Ferro, T. et al. Am. J. Physiol. Lung Cell Mol. Physiol. (2000) 278: L1107-17; Maruo, N. et al. Endocrinology (1992) 131: 710-14]. While leukocytes traditionally have been considered the major source of pro-inflammatory cytokines, lung endothelial cells have been implicated as key regulators of the process, if not the source [Id., citing Teijaro, J R et al. Cell (2011) 146: 980-91]. A small molecule agonist of the sphingosine-1-phosphate receptor (SIP subtype 1) was found to be sufficient to protect against lethal influenza, largely by reducing pro-inflammatory cytokine production, and inhibited the recruitment of neutrophils and macrophages/monocytes to the lung. Impairment of leukocyte recruitment did not account for the blunted cytokine storm, suggesting that endothelial cells may have been the source of the cytokines. SP itself is known to reduce endothelial permeability [Id., citing Garcia, J G et al. J. Clin. Invest. (2001) 108: 689-701].

The recruitment of leukocytes to the lung also has been postulated to be involved in ARDS after influenza. Neutrophils in particular have been implicated in causing lung endothelial damage, through the generation of neutrophil extracellular traps (NETs) [Id., citing Narasaraju, et al. Am. J. Pathol. (2011) 179: 199-210], the secretion of elastase [Id., citing Lee, W L and Downey, G P Am. J. Resp. Crit. Care Med. (2001) 164: 896-904] and the generation of reactive oxygen species [Lee, W L and Downey, G P. Curr. Opin. Crit. Care (2001) 7: 1-7]. In contrast, macrophages appear to be protective against influenza [Id., citing Cao, W. et al. J. Immunol. (2012) 189: 2257-65] since macrophage-depleted mice displayed worsened lung injury and increased neutrophil accumulation in the lung [Id., citing Narasaraju, et al. Am. J. Pathol. (2011) 179: 199-210].

Endothelial activation and barrier function after infection with H5N1 avian influenza virus were postulated to be related to NF-κB. Endothelial cell-specific blockade of NF-κB activation reduces lung edema, neutrophil infiltration, and mortality after E. coli bacteremia or after cecal ligation and perforation, independent of bacterial clearance [Id., citing Xu, H. et al. J. Pathol. (2010) 220: 490-98; Ye, X. et al. J. Exp. Med. (2008) 205: 1303-15]. It has been suggested that excessive endothelial activation leads to endothelial apoptosis in association with decreased survival from sepsis [Id., citing Minami, T. et al. J. Clin. Invest. (2009) 119: 2257-70]. An effect of influenza virus infection on lung endothelial NF-κB could therefore lead to loss of the endothelial barrier. It also has been suggested that cytokines can induce endothelial leak independently of NF-κB [Id., citing Zhu, W. et al. Nature (2012) 492: 252-55].

It has also been hypothesized that endothelial barrier dysfunction could result from direct cytopathic effects of the virus. Human endothelial cells are known to express a2,6-linked sialic acid residues, the receptor for human influenza virus [Id., citing Abe, Y. et al J. Immunol. (1999) 163: 2867-76; Yao, L. et al. FASEB J. (2008) 22: 733-40]; expression increases when endothelial cells are stimulated with cytokines, as might occur in serious infections [Id., citing Hanasaki, K. et al. J. Biol. Chem. (1994) 269: 10637-43]. Influenza virus infection of the lung endothelium has been observed in vitro and causes endothelial cell death [Id., citing Armstrong, S M et al. PLoS One (2012) 7: e47323], cytokine production [Id., citing Visseren, F L et al, J. Lab Clin. Med. (1999) 134: 623-30; Wang, S. et al. J. Infect. Dis. (2010) 202: 991-1001], as well as a decrease in the expression of endothelial cell junctional proteins [Id., citing Armstrong, S M et al. PLoS One (2012) 7: e47323; Wang, S. et al. J. Infect. Dis. (2010) 202: 991-1001] that lead to increased permeability. The relative contribution of direct endothelial infection to the pathogenesis of severe influenza is unknown.

COVID-19

In severe cases of COVID-19, the onset of ARDS begins with viral entry into alveolar pneumocytes, which line the alveoli, the gas exchange units of the lungs (i.e., the same cells that are vulnerable to infection from influenza). Once the virus permeates this sac, the cells will eventually experience apoptosis, triggering cell death in that alveolar unit, as well as adjacent units. This process leads to diffuse alveolar damage (DAD), injuring the capillaries that deliver oxygen to the rest of the body, as well as other cells in the alveoli. The damage to capillaries and other alveolar cells in DAD combines to produce hyaline membranes, a key pathologic feature of the disease. The features of COVID induced acute lung injury are hallmarks of the acute respiratory distress syndrome (ARDS).

It has been reported that one of the main features of ARDS in COVID-19 is an uncontrolled systemic inflammatory response resulting from the release of pro-inflammatory cytokines and chemokines by immune effector cells [Nile, S H et al. Cytokine Growth Factor Rev. (2020). Doi: 10.1016/j.cytogfr.2020.05.002, citing Li, X. et al. J. Pharm. Analysis (2020) doi.org/10.1016/j.jpha.2020.03.001]. High blood levels of cytokines and chemokines have been detected in patients with COVID-19 infection, including: IL1-β, IL1RA, IL7, IL8, IL9, IL10, basic FGF2, GCSF, GMCSF, IFNγ, IP10, MCP1, MIP1α, MIP1β, PDGFB, TNFα, and VEGFA [Id., citing Rothan, H A, et al. J. Autoimmun. (2020) 109 doi/org10.1016/j.jaut.2020.102433]. The ensuing cytokine storm triggers a violent inflammatory immune response that contributes to ARDS, multiple organ failure, and finally death in severe cases of SARS-CoV-2 infection, which is similar to SARS-CoV and MERS-CoV infections [Id., citing Li, X. et al. J. Pharm. Analysis (2020) doi/org10.1016/j.jpha. 2020.03.001]. Patients infected with COVID-19 showed higher leukocyte numbers, abnormal respiratory findings, and increased levels of plasma pro-inflammatory cytokines [Id., citing Huang, C. et al. Lancet (2020) 395 (10223): 4997-506; Sun, X. et al. Cytokine Growth Factor Rev. (2020) doi/org10.1016/j.cytogfr.2020.04.002]. The direct cause of death from acute COVID-19 was reported to involve cytokine storm damage to lungs and multiple organs of the body: heart, kidney and liver, leading to multiple organ exhaustion [Id., citing Mehta, P., et al. Lancet (2020) doi/org10.1016/50140-6736(20)30628-0; Tisoncik, J R et al. Microbiol. Mol. Biol. Rev. (2012) 76: 16-32; Wang, Y. et al. Nat. Immunol. (2014) 15: 1009-16; Wang, G. et al. Cell Death Differ. (2018) 25: 1209-23].

Evidence is accumulating that vascular endothelial injury may be a precipitating factor in severe organ damage caused by COVID [Varga, Z. et al. The Lancet (2020) doi.org/10.1016/S0140-6736(20)30937-5]. Multiple examples of viral invasion of vascular endothelial cells associated with inflammation, endothelial cell death, microvascular dysfunction and organ failure were found. Evidence of vascular leakage in the lungs of COVID-19 patients also were found. Tian et al [Tian S. et al. J. Thoracic Oncology (2020) doi.org/10.1016/j.jtho.2020.02.010) showed histopathological data obtained on the lungs of two patients who underwent lung lobectomies for adenocarcinoma and were retrospectively found to have had COVID-19 infection at the time of surgery. Apart from the tumors, the lungs of both of these cases showed edema and proteinaceous exudates as large protein globules indicating a severe loss of vascular integrity. The authors also reported vascular congestion. These findings are consistent with inflammation and severe vascular damage.

In summary, the predominance of pulmonary manifestations is an important feature of severe COVID-19 infection. To date, reported fatalities have virtually all been accompanied by evidence of pneumonia and systemic inflammation [Zhou, F. et al. Lancet (2020) 395: 1054-62; Pan, Y. et al. Eur. Radiol. (2020) doi.org/10.1007/s00330-020-06731-x; Xu, X. et al. Eur. J. Nuclear Medicine & Molecular Imaging (2020) doi.org/10.1007/s00259-020-04735-9; Xie, J. et al. Intensive Care Med. (2020) doi.org/10.1007/s00134-020-05979-7] In addition, anecdotal evidence indicates that attenuation of inflammation may be beneficial in COVID-19 pneumonia [Zhu, L. et al. Am. J. Transplant. (2020) 00: 1-5] and the acute use of a variety of anti-inflammatory approaches is being explored. However, to our knowledge there is no attempt being made to repair lungs damaged by COVID-19.

After infection with SARS-CoV, the acute lung injury caused by the virus must be repaired to regain lung function; a dysregulation in this wound healing process may lead to fibrosis.

A wound-healing response often is described as having three distinct phases-injury, inflammation and repair. Generally speaking, the body responds to injury with an inflammatory response, which is crucial to maintaining the health and integrity of an organism. If, however, it goes awry, it can result in tissue destruction.

Although these three phases are often presented sequentially, during chronic or repeated injury, these processes function in parallel, placing significant demands on regulatory mechanisms [Wilson and Wynn, Mucosal Immunol., 2009, 3(2): 103-121].

Phase I: Injury

Injury caused by factors including, but not limited to, autoimmune or allergic reactions, environmental particulates, or infection or mechanical damage, often results in the disruption of normal tissue architecture, initiating a healing response. Damaged epithelial and endothelial cells must be replaced to maintain barrier function and integrity and prevent blood loss, respectively. Acute damage to endothelial cells leads to the release of inflammatory mediators and initiation of an anti-fibrinolytic coagulation cascade, temporarily plugging the damaged vessel with a platelet and fibrin-rich clot. In addition, thrombin (a serine protease required to convert fibrinogen into fibrin) is also readily detected within the lung and intra-alveolar spaces of several pulmonary fibrotic conditions, further confirming the activation of the clotting pathway. Thrombin also can directly activate fibroblasts, increasing proliferation and promoting fibroblast differentiation into collagen-producing myofibroblasts. Damage to the airway epithelium, specifically alveolar pneumocytes, can evoke a similar anti-fibrinolytic cascade and lead to interstitial edema, areas of acute inflammation, and separation of the epithelium from the basement membrane.

Platelet recruitment, degranulation and clot formation rapidly progress into a phase of vasoconstriction with increased permeability, allowing the extravasation (movement of white blood cells from the capillaries to the tissues surrounding them) and direct recruitment of leukocytes to the injured site. The basement membrane, which forms the extracellular matrix underlying the epithelium and endothelium of parenchymal tissue, precludes direct access to the damaged tissue. To disrupt this physical barrier, zinc-dependent endopeptidases, also called matrix metalloproteinases (MMPs), cleave one or more extracellular matrix constituents allowing extravasation of cells into, and out of, damaged sites.

Phase II:

Once access to the site of tissue damage has been achieved, chemokine gradients recruit inflammatory cells. Neutrophils, eosinophils, lymphocytes, and macrophages are observed at sites of acute injury with cell debris and areas of necrosis cleared by phagocytes.

The early recruitment of eosinophils, neutrophils, lymphocytes, and macrophages providing inflammatory cytokines and chemokines can contribute to local TGF-β and IL-13 accumulation. Following the initial insult and wave of inflammatory cells, a late-stage recruitment of inflammatory cells may assist in phagocytosis, in clearing cell debris, and in controlling excessive cellular proliferation, which together may contribute to normal healing. Late-stage inflammation may serve an anti-fibrotic role and may be required for successful resolution of wound-healing responses. For example, a late-phase inflammatory profile rich in phagocytic macrophages, assisting in fibroblast clearance, in addition to IL-10-secreting regulatory T cells, suppressing local chemokine production and TGF-β, may prevent excessive fibroblast activation.

The nature of the insult or causative agent often dictates the character of the ensuing inflammatory response. For example, exogenous stimuli like pathogen-associated molecular patterns (PAMPs) are recognized by pathogen recognition receptors, such as toll-like receptors and NOD-like receptors (cytoplasmic proteins that have a variety of functions in regulation of inflammatory and apoptotic responses), and influence the response of innate cells to invading pathogens. Endogenous danger signals also can influence local innate cells and orchestrate the inflammatory cascade.

The nature of the inflammatory response dramatically influences resident tissue cells and the ensuing inflammatory cells. Inflammatory cells themselves also propagate further inflammation through the secretion of chemokines, cytokines, and growth factors. Many cytokines are involved throughout a wound-healing and fibrotic response, with specific groups of genes activated in various conditions. Fibrotic lung disease (such as idiopathic pulmonary fibrosis) patients more frequently present pro-inflammatory cytokine profiles (including, but not limited to, interleukin-1 alpha (IL-1α), interleukin-1 beta (IL-1β), interleukin-6 (IL-6), tumor necrosis factor alpha (TNF-α), transforming growth factor beta (TGF-β), and platelet-derived growth factors (PDGFs)). Each of these cytokines has been shown to exhibit significant pro-fibrotic activity, acting through the recruitment, activation and proliferation of fibroblasts, macrophages, and myofibroblasts.

Phase III: Tissue Repair and Contraction

The closing phase of wound healing consists of an orchestrated cellular reorganization guided by a fibrin (a fibrous protein that is polymerized to form a “mesh” that forms a clot over a wound site)-rich scaffold formation, wound contraction, closure and re-epithelialization. The vast majority of studies elucidating the processes involved in this phase of wound repair have come from dermal wound studies and in vitro systems.

Myofibroblast-derived collagens and smooth muscle actin (α-SMA) form the provisional extracellular matrix, with macrophage, platelet, and fibroblast-derived fibronectin forming a fibrin scaffold. Collectively, these structures are commonly referred to as granulation tissues. Primary fibroblasts or alveolar macrophages isolated from idiopathic pulmonary fibrosis (IPF) patients produce significantly more fibronectin and α-SMA than control fibroblasts, indicative of a state of heightened fibroblast activation. It has been reported that IPF patients undergoing steroid treatment had similar elevated levels of macrophage-derived fibronectin as IPF patients without treatment. Thus, similar to steroid resistant IL-13-mediated myofibroblast differentiation, macrophage-derived fibronectin release also appears to be resistant to steroid treatment, providing another reason why steroid treatment may be ineffective. From animal models, fibronectin appears to be required for the development of pulmonary fibrosis, as mice with a specific deletion of an extra type III domain of fibronectin (EDA) developed significantly less fibrosis following bleomycin administration compared with their wild-type counterparts.

In addition to fibronectin, the provisional extracellular matrix consists of glycoproteins (such as PDGF), glycosaminoglycans (such as hyaluronic acid), proteoglycans and elastin. Growth factor and TGF-β-activated fibroblasts migrate along the extracellular matrix network and repair the wound. Within skin wounds, TGF-β also induces a contractile response, regulating the orientation of collagen fibers. Fibroblast to myofibroblast differentiation, as discussed above, also creates stress fibers and the neo-expression of α-SMA, both of which confer the high contractile activity within myofibroblasts. The attachment of myofibroblasts to the extracellular matrix at specialized sites called the “fibronexus” or “super mature focal adhesions” pull the wound together, reducing the size of the lesion during the contraction phase. The extent of extracellular matrix laid down and the quantity of activated myofibroblasts determines the amount of collagen deposition. To this end, the balance of matrix metalloproteinases (MMPs) to tissue inhibitor of metalloproteinases (TIMPs) and collagens to collagenases vary throughout the response, shifting from pro-synthesis and increased collagen deposition towards a controlled balance, with no net increase in collagen. For successful wound healing, this balance often occurs when fibroblasts undergo apoptosis, inflammation begins to subside, and granulation tissue recedes, leaving a collagen-rich lesion. The removal of inflammatory cells, and especially α-SMA-positive myofibroblasts, is essential to terminate collagen deposition. Interestingly, in IPF patients, the removal of fibroblasts can be delayed, with cells resistant to apoptotic signals, despite the observation of elevated levels of pro-apoptotic and FAS-signaling molecules.

Regenerative Cells of the Lungs

The lung is a highly quiescent tissue, previously thought to have limited reparative capacity and a susceptibility to scarring. It is now known that the lung has a remarkable reparative capacity, when needed, and scarring or fibrosis after lung injury may occur infrequently in scenarios where this regenerative potential is disrupted or limited [Konen, D. N. and Morrisey, E. E., “Lung regeneration: mechanisms, applications and emerging stem cell populations,” Nat. Med. (2014) 20(8): 822-32, citing Beers, M F and Morrisey, E E, “The 3 R's of lung health and disease—repair, remodeling and regeneration,” J. Clin. Invest. (2011) 121: 2065-73; and Wansleeben, C. et al, “Stem cells of the adult lung: their development and role in homeostasis, regeneration and disease,” Wiley Interdiscip. Rev. Dev. Biol. (2013) 2: 131-148]. Thus, the tissues of the lung may be categorized as having facultative progenitor cell populations that can be induced to proliferate in response to injury as well as to differentiate into one or more cell types.

The adult lung comprises at least 40-60 different cell types of endodermal, mesodermal, and ectodermal origin, which are precisely organized in an elaborate 3D structure with regional diversity along the proximal-distal axis. In addition to the variety of epithelial cells, these include cartilaginous cells of the upper airways, airway smooth muscle cells, interstitial fibroblasts, myofibroblasts, lipofibroblasts, and pericytes as well as vascular, microvascular, and lymphatic endothelial cells, and innervating neural cells. The regenerative ability of lung epithelial stem/progenitor cells in the different regions of the lung are thought to be determined not only by their intrinsic developmental potential but also by the complex interplay of permissive or restrictive cues provided by these intimately associated cell lineages as well as the circulating cells, soluble and insoluble factors and cytokines within their niche microenvironment [McQualter & Bertoncello., Stem Cells. 2012 May; 30(5); 811-16].

The crosstalk between the different cell lineages is reciprocal, multidirectional, and interdependent. Autocrine and paracrine factors elaborated by mesenchymal and endothelial cells are required for lung epithelial cell proliferation and differentiation [Yamamoto et al., Dev Biol. 2007 Aug. 1; 308(1): 44-53; Ding et al., Cell. 2011 Oct. 28; 147(3): 539-53], while endothelial and epithelial cell-derived factors also regulate mesenchymal cell proliferation and differentiation, extracellular matrix deposition and remodeling, and adhesion-mediated signaling [Crivellato. Int J Dev Biol. 2011; 55(4-5): 365-75; Grinnell & Harrington. Pulmonary endothelial cell interactions with the extracellular matrix. In: Voelkel N F, Rounds S, eds. The Pulmonary Endothelium: Function in Health and Disease. Chichester, West Sussex: Wiley-Blackwell, 2009: 51-72]. Chemotactic factors elaborated by these cell lineages also orchestrate the recruitment of inflammatory cells, which participate in the remodeling of the niche and the regulation of the proliferation and differentiation of its cellular constituents [McQualter & Bertoncello. Stem Cells. 2012 May; 30(5); 811-16].

CD34+ Cell Therapy for Tissue Repair

Unmodified (i.e., not fractionated) bone marrow or blood-derived cells have been used in several clinical studies, for example, Hamano, K. et al., Japan Cir. J. (2001)65: 845-47; Strauer. B. E., et al., Circulation (2002)106: 1913-18; Assmus, et al., Circulation (2002)106: 3009-3017; Dobert, N. et al., Fur. J. Nuel. Med. Mol. Imaging, (2004)8: 1146-51; Wollert, K. C. et al., Lancet (2004)364: 141-48. Bone marrow consists of a variety of precursor and mature cell types, including hematopoietic cells (the precursors of mature blood cells) and stromal cells (the precursors of a broad spectrum of connective tissue cells), both of which appear to be capable of differentiating into other cell types [Wang, J. S. et al., J. Thorac. Cardiovasc. Surg. (2001)122: 699-705; Tomita, S. et al., Circulation (1999)100 (Suppl. II): 247-256; Saito, T. et al., Tissue Eng. (1995)1: 327-43].

An enriched population of 3.2-4.4×10⁶ autologous bone marrow derived CD34+ cells/kg when transplanted into lethally irradiated baboons was shown to reconstitute granulocyte and platelet counts by 13-24 d; the transplanted bone marrow showed normal cellularity and the presence of all hematopoietic lineages. [Berenson, R J et al. J. Clin. Invest. (1988) 81: 951-55].

CD34 is a hematopoietic stem cell antigen selectively expressed on hematopoietic stem and progenitor cells derived from human bone marrow, blood and fetal liver [Yin et al., Blood (1997) 90: 5002-5012; Miaglia, S. et al., Blood (1997) 90: 5013-21]. Cells that express CD34 are termed CD34+. Stromal cells do not express CD34 and are therefore termed CD34−. In humans, CD34+ cells represent approximately 1% of bone marrow derived nucleated cells; CD34 antigen also is expressed by immature endothelial cell precursors (mature endothelial cells do not express CD34) [Peichev, M. et al., Blood (2000) 95: 952-58].

Asahara et al. first demonstrated that cells isolated with anti-CD34 or anti-Flk-1 from human peripheral blood could differentiate into EC's in vitro and home to foci of angiogenesis in vivo; Flk-1 is also known as VEGFR-2 in mouse [Asahara, T. et al., Science (1997) 275: 964]. Immunomagnetic bead separation was used to isolate a highly purified and viable population of CD34+ cells from human peripheral blood. CD34-depleted cells (CD34-) were used as controls. The CD34+ cells isolated from human blood progressed to an EC-like phenotype; expression of CD34, CD31, Flk-1, Tie-2 and E-selectin, all markers of the EC lineage [Id., citing Millauer, B. et al. Cell (1993) 72: 835; Yamaguchi, D J. Et al. Development (1993) 118: 489; Newman, P J et al. Science (1990) 247: 1219; Sato, T N, et al. Nature (1995) 376: 70; Schnurch, H. and Risau, W. Development (1993): 119: 957; Bevilacqua, M P, Annu. Rev. Immunol. (1993) 11: 767] was greater in attached CD34+ cells (ATc^{D34+}) in culture than in freshly isolated CD34+ mononuclear blood cells (MB^{CD34}+ cells). Expression of ecNOS, Flk-1/KDR (the human homolog of VEGFR-2) and CD31 mRNA at 7, 14, and 21 days was documented by RT-PCR; evidence for ecNOS and Flk-1/KDR in ATc^{D34+} cells was also demonstrated in a functional assay. Mouse and rabbit models of hindlimb ischemia were used to determine whether MBcD³⁴⁺ cells contribute to angiogenesis in vivo; for administration of human MBcD³⁴⁺ cells, C57BL/6AJ×129/SV background athymic nude mice were used. Two days after creating unilateral hindlimb ischemia by excising one femoral artery, mice were injected with 5×10⁵DiI-labeled human MBcD³⁴⁺ or MBcD³⁴⁻ cells into the tail vein. Histologic examination 1-6 weeks later revealed that the DiL-labeled CD34+ cells had homed to foci of angiogenesis; numerous including proliferative DiI-labeled cells in the revascularized ischemic hindlimb. In vivo incorporation of autologous MB^{CD34+} cells into foci of neovascularization was also tested in a rabbit model of unilateral hindlimb ischemia; DiI-labeled cells were localized exclusively to neovascular zones of the ischemic limb and were incorporated into capillaries that consistently expressed CD31 and reacted with BS-1 lectin.

The track record of CD34+ cells for ischemic tissue repair in multiple human indications of severe injury, as well as the supporting scientific evidence in pre-clinical models, is extensive. A large number of animal studies investigating the potential therapeutic utility of CD34 cell therapy have been published, as summarized in Sietsema, W K et al. Circulation J. (2019) 83: 1422-30.

Mobilization alone did not yield significant evidence of tissue repair [Kawamoto, A., et al. 2004, Circulation, 110: 1398-405]. A randomized double-blind, placebo controlled trial of patients diagnosed with ST-segment elevation AMI who had successful reperfusion by percutaneous coronary intervention within 12 hours after onset of symptoms in Germany from 2004-2005 showed that treatment with G-CSF produced a significant mobilization of stem cells but had no influence on infarct size, left ventricular function or coronary restenosis [Zohlnhofer, D. et al. JAMA (2006) 295 (9): 1003-10]. The pattern of mobilization of EPCs and CD34+ cells was studied during heart failure (HF) [Valgimigli, M. et al. Circulation (2004) 110: 1209-12]. Patients with heart failure show endothelial dysfunction, and diminished nitric oxide production, while rate of endothelial apoptosis increases [Id., citing Katz, S D et al. Circulation (1999) 99: 2113-17; Agnoletti, L. et al. Circulation (1999) 100: 1983-91]. Peripheral blood CD34+ cells (n-91) and endothelial progenitor cells (n=41) were studied in heart failure patients and 45 gender and age matched controls. TNF-α and its receptors, VEGF, SDF-1, G-CSF, and B-type natriuretic peptide were also measured. The results showed that CD34+ cells, EPCs, TNFα and its receptors, VEGF, SDF-1, and B-type natriuretic peptide were increased in HF. CD34+ cells and EPC mobilization that occurs in heart failure shows a biphasic response, with elevation and depression in the early and advanced phases, respectively, which could be stage dependent. Exhaustion of progenitor cells in the advanced phases of HF was hypothesized to also contribute to the biphasic bone marrow pattern.

Selected CD34+ cells have been shown to be superior to unselected mononuclear cells, even with dose matching for CD34+ content. In an athymic nude rat model of AMI human CD34+ cells purified by magnetic cell sorting after a 5-day administration of G-CSF given in a number identical to those contained within a mononuclear cell (MNC) product resulted in significantly greater improvement in perfusion and cardiac function, suggesting that non-CD34+ MNCs in the unselected total MNCs adversely affect the potency of isolated CD34+ cells. [Kawamoto. A. et al. Circulation (2006) 114: 2163-2169].

Moreover, clinical studies have provided substantial evidence for safety and efficacy for reversal of tissue damage in humans [see, e.g., Losordo, D W et al., Circulation (2007) 115: 3165-72; Losordo, D W et al. Cir. Res. (2011) 109 (40): 428-36; Poglaj en, G. et al. Cir. Cardiovasc. Interv. (2014) 7: 552-59; Taguchi, A. et al. J. Clin. Invest. (2004) 114 (5): 330-8; Losordo, D W et al. Cir. Cardiovasc. Interv. (2012) 5(6): 821-30; Fujita, Y. et al. Circulation J. 78 (2014) 490-501].

Human clinical studies in multiple ischemia indications for CD34+ cell preparations continue to be developed, for example, in critical limb ischemia (CLI), the end-stage of lower limb ischemia due to atherosclerotic peripheral artery disease (PAD) or vasculitis including thromboangitis obliterans (Buerger's disease). Restriction of blood flow due to arterial stenosis or occlusion often leads patients to complain of muscle pain on walking (intermittent claudication). Any further reduction in blood flow causes ischemic pain at rest. meaning the demand for oxygen cannot be sustained when resting. Ulceration and gangrene may then supervene in the toes, which are the furthest away from the blood supply, and can result in loss of the involved limb if not treated. Therapies for limb ischemia have the goals of collateral development and blood supply replenishment.

Lung Injury

The therapeutic potential of freshly isolated human umbilical cord blood CD34+ progenitor cells was investigated in a mouse model of ALI based on LPS challenge. As a so-called pathogen-associated molecular pattern, LPS is recognized by TLR4, which is up-regulated on bronchial epithelial cells and lung macrophages during LPS-induced ALI and is considered to play a crucial role in innate immune responses [Rittirsch, D. et al. J. Immunol. (2008) 180 (11): 7664-72, citing Medzhitov, R., Janeway, C., J:r. Immunol. Rev. (2000) 173: 89-97; Saito, T. et al. Cell Tissue Res. (2005) 321: 75-88]. The interaction of LPS with TLR4 ultimately leads to release of proinflammatory mediators and the subsequent recruitment of leukocytes into lungs [Id., citing Kabir, K. et al., Shock (2002) 17: 300-3; Speyer, C L et aa. Am. J. Pathol. (2004) 165: 2187-96; Medzhitov, R., Janeway, C., Jr. Immunol. Rev. (2000) 173: 89-97; Abraham, E. et al. J. Immunol. (2000) 165: 2950-54; Reutershan, J. et al. Am. J. Physiol. (2005) 289: L807-L815]. LPS treatment does not cause the severe endothelial and epithelial injury that occurs in humans with ARDS. [Matute-Bello, G. et al. Am. J. Phys. Lung Cell Mol. Physiol. (2008) 395 (3): L379-L399, citing Wieener-Wolf, K E, et al. Am. J. Physiol. Lung Cell Mol. Physiol. (2006) L21-L31].

Mice received a single dose of E. coli LPS by i.p. injection. [Huang, X. et al. PLoS One (2014) 9(2): e88814]. At 3 h post-LPS challenge, the CD34+ cells were transplanted i.v., to mice, while CD34− cells or phosphate buffered saline (PBS) were administered as controls in separate cohorts. The treatment inhibited lung vascular injury evident by decreased lung vascular permeability. Lung inflammation (determined by meloperoxidase activity, neutrophil sequestration and expression of pro-inflammatory mediators) was attenuated in CD34+-treated mice at 26 hr post-LPS challenge compared to controls. Lung inflammation in CD34+ treated mice returned to normal levels at 52 h post-LPS challenge, whereas control mice exhibited persistent lung inflammation.

The therapeutic effect of isolated human peripheral blood CD34+ cells in an ALI rat model, induced by oleic acid (OA) injection was evaluated. OA-induced ALI is a model of fat embolism syndrome, given that OA is a major component of the marrow-derived fat emboli released into the circulation after traumatic bone injury. The ALI produced by OA is relatively transient and resolves over 3 days [Abd-Allah, S. H. et al. Cytotherapy (2015) 17(4): 443-53]. The oleic acid model is probably not as appropriate for studying the pathophysiology of ALI due to sepsis, or therapeutic strategies aimed at modifying host inflammatory responses to reduce the severity of lung injury [Matute-Bello, G. et al. Am. J. Phys. Lung Cell Mol. Physiol. (2008) 395 (3): L379-L399].

Transplantation of about 5×10⁶ immunomagnetic bead-selected CD34+ cells in rats with oleic acid-induced lung injury reportedly improved the arterial oxygen partial pressure (PaO₂) and wet/dry ratio, reduced infiltration of inflammatory cells, and decreased lung vascular permeability as determined by reduced intra-alveolar and interstitial patchy congestion and hemorrhage and decreased interstitial edema compared to controls. Lung inflammation as determined by expression of ICAM-1 and TNFα was attenuated in CD34+ cell treated rats at 6, 24, and 48 hr post-OA challenge, compared with nontreated rats. Moreover, the expression of anti-inflammatory IL-10 was upregulated in the lungs of OA-induced ALI rats after administration of CD34+ cells. Human TNF-α induced protein 6 [TSG-6] gene expression was significantly up-regulated in rats treated with CD34+ cells. The anti-inflammatory effects of the CD34+ cells in this model may be attributed to their activation to secrete TSG-6 [Abd-Allah, S. H. et al. Cytotherapy (2015) 17(4): 443-53].

The ACE2/Ang-1(−7)/Mas pathway stimulates vascular repair-relevant functions of CD34+ cells, while the ACE/Ang II/AT1 axis attenuates these CD34+ cell functions, either directly or indirectly by stimulating the generation of reactive oxygen species from MNCs [Singh, N. et al. Am J. Physiol. Heart Circ. Physiol. (2015) 309 (10): H1697-H1707]. ACE2 and Mas receptor are relatively highly expressed in CD34+ cells compared with MNCs. Ang-(1-7) or its analog, Norleu3-Ang-(1-7) stimulated proliferation of CD34+ cells that was associated with a decrease in phosphatase and tensin homologue deleted on chromosome 10 levels, and was inhibited by triciribine, an AKT inhibitor. Migration of CD34+ cells was enhanced by Ang-(1-7) or Norleu3-Ang-(1-7) that was decreased by Rho-kinase inhibitor, Y-27632. In the presence of Ang II, ACE2 activators xanthenone (XNT) and diminazene (DIZE) enhanced proliferation and migration that were blocked by DX 600, an ACE2 inhibitor. Treatment of MNCs with Ang II, before isolation of CD34+ cells, attenuated their proliferation and migration to stromal derived factor 1α. This attenuation was reversed by apocynin, an NADPH oxidase inhibitor. Adhesion of MNCs or CD34+ cells to fibronectin was enhanced by Ang II and was unaffected by Ang-(1-7).

The fact that CD34+ cells exhibit high levels of ACE2 expression [Singh, N. et al. Am J. Physiol. Heart Circ. Physiol. (2015) 309 (10): H1697-H1707], a key target for COVID-19 cell entry, indicates that depletion of the lung's pool of CD34+ cells may be particularly important in the inability of COVID-19 patients to recover.

Prior data from the SARS epidemic indicated that CD34+ cells in the lung could be a specific target of coronavirus infection, and that destruction of lung CD34+ progenitors could account for the inability of patients with severe pulmonary manifestations to recover. Using triple-color sequential immunohistochemistry and immunofluorescence staining on the same section, SARS-infected cells in the lung of fatally infected patients were shown to express ACE2, a binding receptor, liver/lymph node-specific intercellular adhesion molecule 3 (ICAM-3)-grabbing non-integrin (CD209L), CD34, and Oct-4, and to not express cytokeratin or surfactant [Chen, Y. et al. JEM (2007) (204(1): 2529-36]. Oct-4 is a transcription factor whose activity is essential for maintaining pluripotency of mammalian embryonic cells. [Id., citing Nicols, J, et al. Cell (1998) 95: 379-91; Scholer, H R, et al. EMBO J. (1990) 9: 2185-95]. These putative CD34+Oct-4+ACE2+ lung stem/progenitor cells can also be identified in some non-SARS individuals; they do not express CD15 (SSEA-1, another marker for stem/progenitor cells), did express L-SIGN, a binding receptor for SARS-CoV, and can be infected by SARS-coronavirus ex vivo. Using a four-color sequential immunohistochemistry (IHC) and simultaneous fluorescence in situ hybridization (FISH) and in situ hybridization (ISH), the SARS+ cells were shown to be distinct from cells expressing the macrophage/monocyte-specific marker CD68.

Prior data also indicates that a loss of lung vascular CD34+ cells is associated with increased risk of lung injury. Acute lung injury was induced in wild type and CD34^{-/-} mice by bleomycin administration endotracheally. CD34^{-/-} mice displayed severe weight loss and early mortality compared with WT controls. Ultrastructural analysis of BLM-tested CD34^{-/-} lungs revealed interstitial edema in the alveolar walls and delamination of endothelial cells from the basal lamina. CD34^{-/-} mice exhibited more pronounced evidence of epithelial remodeling in response to infection with influenza A/strain PR8; this was assessed by the quantification of tissue area displaying abnormal alveolar architecture and high cellular density, accompanied by loss of airways space [Lo, B C, et al. Am. J. Respir. Cell Mol. Bio. 57 (6): 651-61]. Bleomycin causes an acute inflammatory injury followed by reversible fibrosis [Matute-Bello, G. et al. Am. J. Phys. Lung Cell Mol. Physiol. (2008) 395 (3): L379-L399]. There is no formation of hyaline membranes. The pathysiopathological relevance of this model to ARDS therefore is unclear [Matute-Bello, G. et al. Am. J. Phys. Lung Cell Mol. Physiol. (2008) 395 (3): L379-L399].

The severe pulmonary manifestations of influenza and COVID-19 that lead to long term disability and death are mediated by inflammation and vascular damage.

The evidence suggests that patients who have a robust ability to mobilize and recruit CD34+ cells in the setting of tissue damage appear to do quite well, while those in whom these mechanisms are not robust do poorly [Werner, N., et al. 2005, N Engl J Med, 353: 999-1007]. Without being limited by theory, their ability to mobilize these cells may be biologically limited.

There is evidence that patients with chronic diseases do have a decrease in circulating CD34+ cell counts, for example patients with diabetes and peripheral artery disease [See, e.g., Fadini, G. P., et al. 2005. J Am Coll Cardiol, 45: 1449-57]. Nevertheless, the mobilization, collection, selection and delivery of CD34+ cells has shown benefit in these patients [Losordo, D. W., et al. (2012). ‘Circ Cardiovasc Interv, 5: 821-30; Fujita, Y., et al. 2014., Circ J, 78: 490-501].

Similarly, a decrease in circulating CD34+ cells has been noted in chronic heart failure [Valgimigli, M., et al. 2004., Circulation, 110: 1209-12], but collection, concentration and delivery of CD34+ in heart failure patients has provided evidence of tissue repair, improved function and improved survival [Vrtovec, B., et al. 2013., Circ Res, 112: 165-73; Poglajen, G., et al. 2014; Circ Cardiovasc Interv, 7: 552-9].

Accordingly, the described invention provides a clinical trial designed to evaluate autologous CD34+ cell therapy for repair of a lung injury derived from severe virus induced lung damage mediated by inflammation and vascular damage.

### SUMMARY OF THE INVENTION

The described invention provides a method for treating a subject at risk for a lung injury derived from a severe virus infection comprising (a) receiving a subcutaneous injection of a bone marrow stimulant to mobilize CD34+ cells into the peripheral blood; (b) harvesting CD34+ cells from the peripheral blood by apheresis; (c) selecting CD34+ cells by positive selection; (d) formulating a CLBS119 cell product by suspending the selected CD34+ cells in an isotonic solution with serum ranging from 5% to 40%, inclusive and human serum albumin ranging from 0.5-10%, inclusive, to form a pharmaceutical composition; and (e) administering the cell product to the subject; wherein the sterile pharmaceutical composition comprising a therapeutic amount of a mobilized nonexpanded, isolated population of autologous mononuclear cells enriched for CD34+ cells with purity ranging from 55% to 100%, inclusive, which further contains a subpopulation of potent CD34+/CXCR4+ cells; and wherein, the mobilized nonexpanded, isolated population of autologous mononuclear cells enriched for CD34+ cells with purity ranging from 55% to 100%, inclusive, which further contains a subpopulation of potent CD34+/CXCR4+ cells when tested in vitro after passage through an infusion catheter after acquisition: (i) has CXCR-4 mediated chemotactic activity and moves in response to SDF-1; (ii) can form hematopoietic colonies; and (iii) is at least 80% viable.

According to some embodiments, the serum is autologous serum or allogeneic AB negative serum. According to some embodiments, in the absence of serum, from 5% to 20%, inclusive human serum albumin can substitute for serum. According to some embodiments, the lung injury comprises severe lung damage marked by one or more of inflammation, loss of lung endothelial cells/integrity and destruction of the lung microvasculature. According to some embodiments, the method modulates one or more outcomes selected from: pulmonary function; diffusing capacity of the lungs; oxygen saturation, inventory of COVID-19 related symptoms, radiographic evidence of pulmonary infiltrates; duration of use of oxygen, time to clinical improvement (TTCI), time to clinical recovery (TTCR), length of time in ICU, length of time in hospital; or all-cause mortality, compared to a normal healthy control and a placebo control. According to some embodiments, the administering is by infusion, and rate of infusion ranges from 0.5 to 2.0 mL/min.

According to some embodiments, the therapeutic amount is an amount ranging from about 50×10⁶, to about 1000×10⁶ inclusive, i.e., 51×10⁶, 52×10⁶, 53×10⁶, 54×10⁶, 55×10⁶, 56×10⁶, 57×10⁶, 58×10⁶, 59×10⁶, 60×10⁶, 61×10⁶, 62×10⁶ 63×10⁶, 64×10⁶, 65×10⁶, 66×10⁶, 67×10⁶, 68×10⁶, 69×10⁶, 70×10⁶, 71×10⁶, 72×10⁶, 73×10⁶, 74×10⁶, 75×10⁶, 76×10⁶, 77×10⁶, 78×10⁶, 79×10⁶, 80×10⁶, 81×10⁶, 82×10⁶, 83×10⁶, 84×10⁶, 85×10⁶, 86×10⁶, 87×10⁶, 88×10⁶, 89×10⁶, 90×10⁶, 91×10⁶, 92×10⁶, 93×10⁶, 94×10⁶, 95×10⁶, 96×10⁶, 97×10⁶, 98×10⁶, 99×10⁶, 100×10⁶; 110×10⁶, 120×10⁶, 130×10⁶, 140×10⁶, 150×10⁶, 160×10⁶, 170×10⁶, 180×10⁶, 190×10⁶, 200×10⁶, 210×10⁶, 220×10⁶, 230×10⁶, 240×10⁶, 250×10⁶, 260×10⁶, 270×10⁶, 280×10⁶, 290×10⁶, 300×10⁶, 310×10⁶, 320×10⁶, 330×10⁶, 340×10⁶, 350×10⁶, 360×10⁶, 370×10⁶, 380×10⁶, 390×10⁶, 400×10⁶, 410×10⁶, 420×10⁶, 430×10⁶, 440×10⁶, 450×10⁶, 460×10⁶, 470×10⁶, 480×10⁶, 490×10⁶, 500×10⁶, 510×10⁶, 520×10⁶, 530×10⁶, 540×10⁶, 550×10⁶, 560×10⁶, 570×10⁶, 580×10⁶, 590×10⁶, 600×10⁶, 610×10⁶, 620×10⁶, 630×10⁶, 640×10⁶, 650×10⁶, 660×10⁶, 670×10⁶, 680×10⁶, 690×10⁶, 700×10⁶; 710×10⁶, 720×10⁶, 730×10⁶, 740×10⁶, 750×10⁶, 760×10⁶, 770×10⁶, 780×10⁶, 790×10⁶, 800×10⁶, 810×10⁶, 820×10⁶, 830×10⁶, 840×10⁶, 850×10⁶, 860×10⁶, 870×10⁶, 880×10⁶, 890×10⁶, 900×10⁶; 910×10⁶, 920×10⁶, 930×10⁶, 940×10⁶, 950×10⁶, 960×10⁶, 970×10⁶, 980×10⁶, 990×10⁶, or 1000×10⁶ CD34+ cells.

According to some embodiments, the subpopulation of potent CD34+/CXCR4+ cells in the composition contains at least 0.1×10⁶ cells.

According to some embodiments, the subject at risk is a subject who has one or more predisposing factors to the development of lung injury following a severe virus infection. According to some embodiments, the predisposing factors include the very young, the elderly, those with pre-existing health conditions, such as chronic cardiopulmonary or renal disease; diabetes, immunosuppression, severe anemia, an existing illness, and those who are physically weak. According to some embodiments, (a) the subject at risk was diagnosed with COVID-19 and is currently hospitalized for treatment of pulmonary manifestations of the severe virus infection; or (b) the subject at risk received ventilative support during the severe virus infection; or (c) the subject at risk further displays cardiovascular complications; or (d) the subject at risk further comprises evidence for ongoing pulmonary involvement; or (e) the subject at risk comprises biomarker evidence for ongoing inflammation. According to some embodiments, the biomarker evidence comprises a modulated level of one or more of C-reactive protein; troponin, white blood cell count; lymphocyte count; lactate dehydrogenase; tumor necrosis factor alpha; IL-1, IL-6, IL-12, one or more interferon(s), compared to a normal healthy control or a control that has not been treated with the cell product. According to some embodiments, the severe lung infection is caused by influenza or a human coronavirus. According to some embodiments, the human coronavirus is SARSCoV-2. According to some embodiments, the lung injury comprises acute respiratory failure. According to some embodiments, the acute respiratory failure comprises an acute lung injury or acute respiratory distress syndrome. According to some embodiments, the acute lung injury comprises acute onset of diffuse bilateral pulmonary infiltrates by chest radiograph; a PaO2/FiO2≤300 and a pulmonary artery wedge pressure (PAWP)≤18. According to some embodiments, the acute lung injury comprises one or more of acute inflammation, loss of alveolar-capillary membrane integrity, excessive transepithelial neutrophil migration, and release of pro-inflammatory mediators. According to some embodiments, the acute respiratory distress syndrome comprises acute onset of diffuse bilateral pulmonary infiltrates by chest radiograph; a PaO2/FiO2≤200 and a pulmonary artery wedge pressure (PAWP)≤18. According to some embodiments, the proinflammatory mediators include one or more of von Willebrand factor (vWf) antigen, intracellular adhesion molecule-1 (ICAM-1), surfactant protein D (SP-D), receptor for advanced glycation end-products (RAGE), IL-6, IL-8, TNF-α, protein C, or plasminogen activator inhibitor-1. According to some embodiments, RAGE and SP-D are biomarkers for lung epithelial injury. According to some embodiments, neutrophil elastase is a marker for excessive transepithelial neutrophil migration. According to some embodiments, the acute respiratory distress syndrome comprises one or more of diffuse alveolar damage (DAD), alveolar inflammation, or infiltration of neutrophils in the alveoli and distal bronchioles. According to some embodiments, a microvascular endothelial injury with increased release of vWf antigen, upregulation of ICAM-1 or both is indicative of progression to increased capillary permeability. According to some embodiments, the pharmaceutical composition is efficacious to repair the lung injury, restore lung function, reduce scarring or fibrosis or a combination thereof. According to some embodiments, the method is efficacious to improve progression-free survival, overall survival or both. According to some embodiments, the pharmaceutical composition i efficacious to restore a CD34+ cell pool in the lung, lung vascular CD34+ cells, or both. According to some embodiments, the pharmaceutical composition attenuates the IL-6 and IL-8 inflammatory response associated with acute lung injury. According to some embodiments, the pharmaceutical composition modulates platelet and neutrophil deposition, leukocyte accumulation in lung microvessels. According to some embodiments, crosstalk between the CD34+ cells and the lung tissue promotes repair of the lung injury. According to some embodiments, the crosstalk is a paracrine effect. According to some embodiments, the paracrine effect is mediated by paracrine factors elaborated by the CD34+ cells. According to some embodiments, the repair comprises reduced apoptosis of vascular endothelial cells, lung endothelial cells, or lung epithelial cells, or increased angiogenesis or both.

### BRIEF DESCRIPTION OF THE DRAWINGS

FIG. 1 shows a hypothesis of the relationship between SARS-CoV-2 and cell pyroptosis according to Yang, Y. et al. J. Autoimmunity (2020) doi.org/10.1016/j.jaut.2020.102434. COVID-19 may be linked to cell pyroptosis, especially in lymphocytes through the activation of the NLRP3 inflammasome.

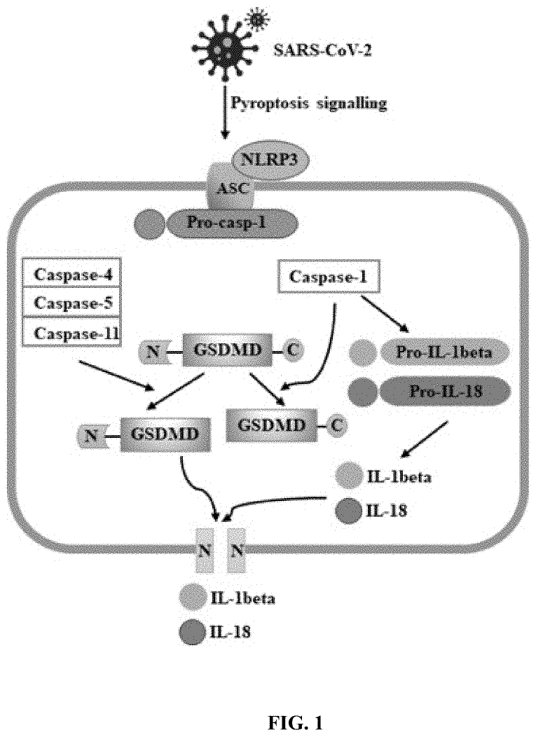

### DETAILED DESCRIPTION

### Glossary

The term “adaptive immunity” as used herein refers to specific, delayed and longer-lasting response by various types of cells that create long-term immunological memory against a specific antigen. It can be further subdivided into cellular and humoral branches, the former largely mediated by T cells and the latter by B cells. This arm further encompasses cell lineage members of the adaptive arm that have effector functions in the innate arm, thereby bridging the gap between the innate and adaptive immune response.

The term “administering” as used herein includes in vivo administration, as well as administration directly to tissue ex vivo. Generally, compositions may be administered systemically (e.g., orally, buccally, parenterally, by inhalation or insufflation (i.e., through the mouth or through the nose), or rectally in dosage unit formulations containing conventional nontoxic pharmaceutically acceptable carriers, adjuvants, and vehicles as desired.

The term “allogeneic” as used herein refers to being genetically different although belonging to or obtained from the same species; e.g., where a donor and a recipient are not the same person.

The term “alveolus” (plural alveoli) as used herein refers to tiny air sacs within the lungs where the exchange of oxygen and carbon dioxide takes place.

The term “angiogenesis” as used herein refers to the process by which new blood vessels take shape from existing blood vessels by “sprouting” of endothelial cells, thus expanding the vascular tree.

The term “angiopoietin” as used herein refers to a family of peptide growth factors that includes the glycoproteins angiopoietin 1 and 2 and the orthologs 3 (in the mouse) and 4 (in humans). Angiopoietins (Ang1, 2, and 4) interact with the Tie2/TEK receptor (RTK), which is preferentially expressed by endothelial cells and some myeloid cells. Ang1 emanates from perivascular tissues and serves as the main Tie2 agonist to stabilize endothelial-mural cell interactions and to promote endothelial cell survival, vascular quiescence, and the nonpermeable state. Ang2, which is produced by VEGF-stimulated endothelium, exerts the opposite effect and stimulates pericyte detachment, permeability, vascular growth, or regression, as well as lymphangiogenesis [Rak, J., Vascular growth in health & disease, in Hematology, 7th Ed. Hoffman, R. et al., Elsevier (2017), Chapter 15].

The term “apoptosis” as used herein refers to a form of cell death characterized by nuclear DNA degradation, nuclear degeneration and condensation, and the rapid phagocytosis of cell remains.

The terms “apoptosis” or “programmed cell death” refer to a highly regulated and active process that contributes to biologic homeostasis comprised of a series of biochemical events that lead to a variety of morphological changes, including blebbing, changes to the cell membrane, such as loss of membrane asymmetry and attachment, cell shrinkage, nuclear fragmentation, chromatin condensation, and chromosomal DNA fragmentation, without damaging the organism.

Apoptotic cell death is induced by many different factors and involves numerous signaling pathways, some dependent on caspase proteases (a class of cysteine proteases) and others that are caspase independent. It can be triggered by many different cellular stimuli, including cell surface receptors, mitochondrial response to stress, and cytotoxic T cells, resulting in activation of apoptotic signaling pathways

The caspases involved in apoptosis convey the apoptotic signal in a proteolytic cascade, with caspases cleaving and activating other caspases that then degrade other cellular targets that lead to cell death. The caspases at the upper end of the cascade include caspase-8 and caspase-9. Caspase-8 is the initial caspase involved in response to receptors with a death domain (DD) like Fas.

Receptors in the TNF receptor family are associated with the induction of apoptosis, as well as inflammatory signaling. The Fas receptor (CD95) mediates apoptotic signaling by Fas-ligand expressed on the surface of other cells. The Fas-FasL interaction plays an important role in the immune system and lack of this system leads to autoimmunity, indicating that Fas-mediated apoptosis removes self-reactive lymphocytes. Fas signaling also is involved in immune surveillance to remove transformed cells and virus infected cells. Binding of Fas to oligimerized FasL on another cell activates apoptotic signaling through a cytoplasmic domain termed the death domain (DD) that interacts with signaling adaptors including FAF, FADD and DAX to activate the caspase proteolytic cascade. Caspase-8 and caspase-10 first are activated to then cleave and activate downstream caspases and a variety of cellular substrates that lead to cell death.

Mitochondria participate in apoptotic signaling pathways through the release of mitochondrial proteins into the cytoplasm. Cytochrome c, a key protein in electron transport, is released from mitochondria in response to apoptotic signals, and activates Apaf-1, a protease released from mitochondria. Activated Apaf-1 activates caspase-9 and the rest of the caspase pathway. Second mitochondria-derived activator of caspase/direct inhibitor of apoptosis-binding protein with low pI [Smac/DIABLO] is released from mitochondria and inhibits IAP proteins that normally interact with caspase-9 to inhibit apoptosis. Apoptosis regulation by Bch 2 family proteins occurs as family members form complexes that enter the mitochondrial membrane, regulating the release of cytochrome c and other proteins. TNF family receptors that cause apoptosis directly activate the caspase cascade, but can also activate Bid, a Bcl-2 family member, which activates mitochondria-mediated apoptosis. Bax, another Bcl-2 family member, is activated by this pathway to localize to the mitochondrial membrane and increase its permeability, releasing cytochrome c and other mitochondrial proteins. Bcl-2 and Bcl-xL prevent pore formation, blocking apoptosis. Like cytochrome c, AIF (apoptosis-inducing factor) is a protein found in mitochondria that is released from mitochondria by apoptotic stimuli. While cytochrome C is linked to caspase-dependent apoptotic signaling, AIF release stimulates caspase-independent apoptosis, moving into the nucleus where it binds DNA. DNA binding by AIF stimulates chromatin condensation, and DNA fragmentation, perhaps through recruitment of nucleases.

The mitochondrial stress pathway begins with the release of cytochrome c from mitochondria, which then interacts with Apaf-1, causing self-cleavage and activation of caspase-9. Caspase-3, -6 and -7 are downstream caspases that are activated by the upstream proteases and act themselves to cleave cellular targets.

Granzyme B and perforin proteins released by cytotoxic T cells induce apoptosis in target cells, forming transmembrane pores, and triggering apoptosis, perhaps through cleavage of caspases, although caspase-independent mechanisms of Granzyme B mediated apoptosis have been suggested.

Fragmentation of the nuclear genome by multiple nucleases activated by apoptotic signaling pathways to create a nucleosomal ladder is a cellular response characteristic of apoptosis. One nuclease involved in apoptosis is DNA fragmentation factor (DFF), a caspase-activated DNAse (CAD). DFF/CAD is activated through cleavage of its associated inhibitor ICAD by caspases proteases during apoptosis. DFF/CAD interacts with chromatin components such as topoisomerase II and histone H1 to condense chromatin structure and perhaps recruit CAD to chromatin. Another apoptosis activated protease is endonuclease G (EndoG). EndoG is encoded in the nuclear genome but is localized to mitochondria in normal cells. EndoG may play a role in the replication of the mitochondrial genome, as well as in apoptosis. Apoptotic signaling causes the release of EndoG from mitochondria. The EndoG and DFF/CAD pathways are independent since the EndoG pathway still occurs in cells lacking DFF.

Hypoxia, as well as hypoxia followed by reoxygenation can trigger cytochrome c release and apoptosis. Glycogen synthase kinase (GSK-3) a serine-threonine kinase ubiquitously expressed in most cell types, appears to mediate or potentiate apoptosis due to many stimuli that activate the mitochondrial cell death pathway. [Loberg, R D, et al., J. Biol. Chem. (2002) 277 (44): 41667-673]. It has been demonstrated to induce caspase 3 activation and to activate the proapoptotic tumor suppressor gene p53. It also has been suggested that GSK-3 promotes activation and translocation of the proapoptotic Bcl-2 family member, Bax, which, upon aggregation and mitochondrial localization, induces cytochrome c release. Akt is a critical regulator of GSK-3, and phosphorylation and inactivation of GSK-3 may mediate some of the antiapoptotic effects of Akt.

The term “apoptosome” as used herein refers to a large multimeric protein structure that forms in the process of apoptosis when cytochrome c is released from mitochondria and binds Apaf-1. A heptamer of cytochrome c-Apaf-1 heterodimers assembles into wheel-like structure that binds and activates procaspase-9, an initiator caspase, to initiate the caspase cascade.

The term “attenuate” as used herein refers to reducing the force, effect, or value of

The term “autologous” as used herein means derived from the same organism.

The term “biocompatible” as used herein refers to a material that is generally non-toxic to the recipient, does not possess any significant untoward effects to the subject and, further, that any metabolites or degradation products of the material are non-toxic to the subject. Typically a substance that is “biocompatible” causes no clinically relevant tissue irritation, injury, toxic reaction, or immunological reaction to living tissue.

The term “biomarkers” (or “biosignatures”) as used herein refers to peptides, proteins, nucleic acids, antibodies, genes, metabolites, or any other substances used as indicators of a biologic state. It is a characteristic that is measured objectively and evaluated as a cellular or molecular indicator of normal biologic processes, pathogenic processes, or pharmacologic responses to a therapeutic intervention. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (choices of drug treatment or administration regimes). In evaluating potential drug therapies, a biomarker may be used as a surrogate for a natural endpoint, such as survival or irreversible morbidity. If a treatment alters the biomarker, and that alteration has a direct connection to improved health, the biomarker may serve as a surrogate endpoint for evaluating clinical benefit. Clinical endpoints are variables that can be used to measure how patients feel, function or survive. Surrogate endpoints are biomarkers that are intended to substitute for a clinical endpoint; these biomarkers are demonstrated to predict a clinical endpoint with a confidence level acceptable to regulators and the clinical community.

The term “carrier” as used herein describes a material, compound or agent that may be contained in a formulation that does not cause significant irritation to an organism and does not abrogate the biological activity and properties of the bioactive agent that may be contained in a composition. A carrier must be of sufficiently high purity and of sufficiently low toxicity to render them suitable for administration to the mammal being treated. The carrier can be inert, or it can possess pharmaceutical benefits. The terms “excipient”, “carrier”, or “vehicle” are used interchangeably to refer to carrier materials suitable for formulation and administration of pharmaceutically acceptable compositions described herein. Carriers and vehicles useful herein include any such materials know in the art which are nontoxic and do not interact with other components. The term includes a single such compound and is also intended to include a plurality of such compounds.

The term “complication” as used herein refers to a pathological process or event during a disorder that is not an essential part of the disease, although it may result from it or from independent causes. A delayed complication is one that occurs some time after a triggering event or effect.

The term “composition” as used herein refers to a mixture of ingredients.

The term “CXCR-7” as used herein refers to a CXC membrane-associated chemokine receptor that binds to stromal-derived factor-1 (SDF-1). CXCR-7 also binds interferon-inducible T-cell chemoattractant (I-TAC) (CXCL11); I-TAC activates CXCR-7 but not CXCR-4. In human T lymphocytes or CD34+ progenitors, CXCR-7 has been implicated in modulating the CXCL12-CXCR-4 signaling axis. Without being limited by theory, it has been suggested that CXCR-7 cross-talk with CXCR-4 is essential for rapid CXCL12 triggered activation involved in lymphocyte and CD34+ cell arrest on endothelial surfaces expressing integrin ligands for CXCR-4 to maintain critical adhesiveness to CXCL-12, without which rapid downstream signaling cannot proceed. [Hartmann, T N, et al., J. Leukoc. Biology 84: 1130-1139 (2008)].

The term “CLBS119 cell product” as used herein refers to a sterile pharmaceutical composition comprising a nonexpanded, isolated population of autologous mononuclear cells enriched for CD34+ cells so that purity of CD34+ cells is 55% to 100%, inclusive, i.e., 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as determined by flow cytometry, which further contains a subpopulation of potent CD34+/CXCR4+ cells that, when tested in vitro after passage through a catheter after acquisition: (i) have CXCR-4 mediated chemotactic activity and move in response to SDF-1; (ii) can form hematopoietic colonies; and (iii) are at least 80% viable. According to some embodiments, viability can be determined by fluorescence-based live dead staining by flow cytometry, fluorescence microscopy or microplate assay.

The term “complement” as used herein refers to a system of soluble pattern recognition receptors and effector molecules that detect and destroy microorganisms. In the presence of pathogens or of antibody bound to pathogens, soluble plasma proteins that in the absence of infection circulate in an inactive form becomes activated, so that particular complement proteins interact with each other to form the pathways of complement activation, which are initiated in different ways. The classical pathway is initiated when complement component C1, which comprises a recognition protein (C1q) associated with proteases (C1r and C1s) either recognizes a microbial surface directly or binds to antibodies already bound to a pathogen. The alternative pathway can be initiated by spontaneous hydrolysis and activation of complement component C3, which can then bind directly to microbial surfaces. The lectin pathway is initiated by soluble carbohydrate-binding proteins-mannose-binding lectin (MBL) and the ficolins-that bind to particular carbohydrate structures on microbial surfaces. MBL-associated serine proteases (MASPs), which associate with these recognition proteins, then trigger cleavage of complement proteins and activation of the pathway. These three pathways converge at the step whereby enzymatic activity of a C3 convertase is generated. The C3 convertase is bound covalently to the pathogen surface, where it cleaves C3 to generate large amounts of C3b, the main effector molecule of the complement system, and C3a, a small peptide that binds to specific receptors and helps induce inflammation. Cleavage of C3 is the critical step in complement activation and leads directly or indirectly to all the effector activities of the complement system. All three pathways have the final outcome of killing the pathogen, either directly or by facilitating its phagocytosis, and inducing inflammatory responses that help to fight infection.

Besides acting in innate immunity, the complement system also influences adaptive immunity. For example, opsonization of pathogens (meaning the coating of the surface of a pathogen that makes it more easily ingested by phagocytes) by complement facilitates their uptake by phagocytic APCs that express complement receptors, which enhances presentation of pathogen antigens to T cells. B cells express receptors for complement proteins that enhance their responses to complement-coated antigens. Several complement fragments also can act to influence cytokine production by APCs, thereby influencing the direction and extent of the subsequent adaptive immune response. [Janeway's Immunology, 9th Ed. 2017, Garland Science, New York, Chapter 2, 49-51].

The term “cytokine” as used herein refers to small soluble protein substances secreted by cells which have a variety of effects on other cells. Cytokines mediate many important physiological functions including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (“TNF”)-related molecules, including TNF a and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines.

The term “diffuse alveolar damage (DAD)” represents a global injury to the gas-exchange surfaces of the lung that is caused by disruption of the blood-air barrier leading to exudative edema and fibrosis, and resulting in severely impaired blood and tissue oxygenation.

The term “derived from” as used herein is meant to encompass any method for receiving, obtaining, or modifying something from a source of origin.

The term “donor” as used herein refers to one who gives or donates.

The term “effective treatment” as used herein refers to one that provides improvement in the general health of a subject.

The term “efficacious treatment’ as used herein refers to one that results in an outcome judged more beneficial than that which would exist without treatment. The term “endothelial activation” as used herein refers to changes to the endothelium under the stimulation of agents that allow it to participate in the inflammatory response. [Hunt, B. J., K. M. Jurd, B M J (1998) 316 (7141): 1328-29]. The five core changes of endothelial cell activation are loss of vascular integrity; expression of leucocyte adhesion molecules; change in phenotype from antithrombotic to prothrombotic; cytokine production; and upregulation of HLA molecules. Loss of vascular integrity can expose subendothelium and cause the efflux of fluids from the intravascular space. Upregulation of leucocyte adhesion molecules such as E-selectin, ICAM-1, and VCAM-1 allows leucocytes to adhere to endothelium and then move into the tissues [Id., citing Adams, D H, Shaw, S. Lancet (1994) 343: 831-36]. The prothrombotic effects of endothelial cell activation include loss of the surface anticoagulant molecules thrombomodulin and heparan sulfate; reduced fibrinolytic potential due to enhanced plasminogen activator inhibitor type 1 release; loss of the platelet anti-aggregatory effects of ecto-ADPases and prostacyclin; and production of platelet activating factor, nitric oxide, and expression of tissue factor [Id., citing Bach, F H et al. Nature Medicine (1995) 1: 869-73]. Cytokines are synthesised, including interleukin [Id., citing Pober, J S, et al. Transplantation (1996) 61: 343-49], which regulates the acute phase response, and chemoattractants such as interleukin [Id., citing Rajavashisth, T B et al. Arterioscler. Thromb. Vasc. Bio. (1995) 15: 1591-98] and monocyte chemoattractant protein 1 [Id., citing Mantovani, A. et al. Thromb. Haemost. (1997) 78: 406-14]. Expression of class II HLA molecules allows endothelial cells to act as antigen presenting cells [Id., citing Pober, J S, et al. Transplantation (1996) 61: 343-49].

Two stages of endothelial cell activation exist [Id., citing Bach, F H et al. Nature Medicine (1995) 1: 869-73]; the first, endothelial cell stimulation or endothelial cell activation type I, does not require de novo protein synthesis or gene upregulation and occurs rapidly. Effects include the retraction of endothelial cells, expression of P selectin, and release of von Willebrand factor. The second response, endothelial cell activation type II, requires time for the stimulating agent to cause an effect via gene transcription and protein synthesis. The genes involved are those for adhesion molecules, cytokines, and tissue factor. is induced by a wide range of agents such as certain bacteria and viruses, interleukin 1 and tumor necrosis factor, physical and oxidative stress, oxidized low density lipoproteins [Id., citing Rajavashisth, T B et al. Arterioscler. Thromb. Vasc. Bio. (1995) 15: 1591-98], and anti-endothelial cell antibodies (found in systemic autoimmune diseases such as the vasculitides, systemic lupus erythematosis, and antiphospholipid syndrome [Id., citing Meroni, P. et al. Lupus (1995) 4: 95-99]. Endothelial cell activation is a graded rather than an all or nothing response—for example, changes in endothelial cell integrity range from simple increases in local permeability to major endothelial cell contraction, exposing large areas of subendothelium. Activation may occur locally, as in transplant rejection [Id., citing Bach, F H et al. Nature Medicine (1995) 1: 869-73], or systemically, as in septicemia and the systemic inflammatory response.

As used herein, the term “enrich” is meant to refer to increasing the proportion of a desired substance, for example, to increase the relative frequency of a subtype of cell or cell component compared to its natural frequency in a cell population. Positive selection, negative selection, or both are generally considered necessary to any enrichment scheme. Selection methods include, without limitation, magnetic separation and fluorescence-activated cell sorting (FACS).

The term “expand” and its various grammatical forms as used herein refers to a process by which dispersed living cells propagate in vitro in a culture medium that results in an increase in the number or amount of viable cells.

The term “factors” as used herein refers to nonliving components that have a chemical or physical effect. For example, a “paracrine factor” is a diffusible signaling molecule that is secreted from one cell type that acts locally on another cell type in a tissue. A “transcription factor” is a protein that binds to specific DNA sequences and thereby controls the transfer of genetic information from DNA to mRNA.

The term “fibrosis” as used herein refers to the formation or development of excess fibrous connective tissue in an organ or tissue as a result of injury or inflammation of a part, or of interference with its blood supply. It may be a consequence of the normal healing response leading to a scar, or it may be an abnormal, reactive process.

“Ground-glass opacity” (GGO) is a radiological finding in computed tomography (CT) consisting of a hazy opacity that does not obscure the underlying bronchial structures or pulmonary vessels [Kobayashi, Y., Mitsudomi, T., Transl. Lung Cancer Res. (2013) 2(5): 354-63, citing Hansell D M, et al. Fleischner Society: glossary of terms for thoracic imaging. Radiology (2008) 246:697-722]. Pure GGOs are those with no solid components, whereas part-solid GGOs contain both GGO and a solid component. GGO can be a manifestation of a wide variety of clinical features, including malignancies and benign conditions, such as focal interstitial fibrosis, inflammation, and hemorrhage [Id., citing Park C M, et al. Nodular ground-glass opacity at thin-section CT: histologic correlation and evaluation of change at follow-up. Radiographics (2007) 27:391-408].

The term “growth factor” as used herein refers to extracellular polypeptide molecules that bind to a cell-surface receptor triggering an intracellular signaling pathway, leading to proliferation, differentiation, or other cellular response. These pathways stimulate the accumulation of proteins and other macromolecules, e.g., by increasing their rate of synthesis, decreasing their rate of degradation, or both.

Fibroblast Growth Factor (FGF). The fibroblast growth factor (FGF) family currently has over a dozen structurally related members. FGF1 is also known as acidic FGF; FGF2 is sometimes called basic FGF (bFGF); and FGF7 sometimes goes by the name keratinocyte growth factor. Over a dozen distinct FGF genes are known in vertebrates; they can generate hundreds of protein isoforms by varying their RNA splicing or initiation codons in different tissues. FGFs can activate a set of receptor tyrosine kinases called the fibroblast growth factor receptors (FGFRs). Receptor tyrosine kinases are proteins that extend through the cell membrane. The portion of the protein that binds the paracrine factor is on the extracellular side, while a dormant tyrosine kinase (i.e., a protein that can phosphorylate another protein by splitting ATP) is on the intracellular side. When the FGF receptor binds an FGF (and only when it binds an FGF), the dormant kinase is activated, and phosphorylates certain proteins within the responding cell, activating those proteins.

FGFs are associated with several developmental functions, including angiogenesis (blood vessel formation), mesoderm formation, and axon extension. While FGFs often can substitute for one another, their expression patterns give them separate functions. For example, FGF2 is especially important in angiogenesis, whereas FGF8 is involved in the development of the midbrain and limbs.

Insulin-Like Growth Factor (IGF-1). IGF-1, a hormone similar in molecular structure to insulin, has growth-promoting effects on almost every cell in the body, especially skeletal muscle, cartilage, bone, liver, kidney, nerves, skin, hematopoietic cell, and lungs. It plays an important role in childhood growth and continues to have anabolic effects in adults. IGF-1 is produced primarily by the liver as an endocrine hormone as well as in target tissues in a paracrine/autocrine fashion. Production is stimulated by growth hormone (GH) and can be retarded by undernutrition, growth hormone insensitivity, lack of growth hormone receptors, or failures of the downstream signaling molecules, including tyrosine-protein phosphatase non-receptor type 11 (also known as SHP2, which is encoded by the PTPN11 gene in humans) and signal transducer and activator of transcription 5B (STAT5B), a member of the STAT family of transcription factors. Its primary action is mediated by binding to its specific receptor, the Insulin-like growth factor 1 receptor (IGF1R), present on many cell types in many tissues. Binding to the IGF1R, a receptor tyrosine kinase, initiates intracellular signaling; IGF-1 is one of the most potent natural activators of the AKT signaling pathway, a stimulator of cell growth and proliferation, and a potent inhibitor of programmed cell death. IGF-1 is a primary mediator of the effects of growth hormone (GH). Growth hormone is made in the pituitary gland, released into the blood stream, and then stimulates the liver to produce IGF-1. IGF-1 then stimulates systemic body growth. In addition to its insulin-like effects, IGF-1 also can regulate cell growth and development, especially in nerve cells, as well as cellular DNA synthesis.

IGF-1 was shown to increase the expression levels of the chemokine receptor CXCR4 (receptor for stromal cell-derived factor-1, SDF-1) and to markedly increase the migratory response of MSCs to SDF-1 [Li, Y, et al. 2007 Biochem. Biophys. Res. Communic. 356(3): 780-784]. The IGF-1-induced increase in MSC migration in response to SDF-1 was attenuated by PI3 kinase inhibitor (LY294002 and wortmannin) but not by mitogen-activated protein/ERK kinase inhibitor PD98059. Without being limited by any particular theory, the data indicate that IGF-1 increases MSC migratory responses via CXCR4 chemokine receptor signaling which is PI3/Akt dependent.

Transforming Growth Factor Beta (TGF-β). There are over 30 structurally related members of the TGF-β superfamily, and they regulate important interactions in development. The proteins encoded by TGF-β superfamily genes are processed such that the carboxy-terminal region contains the mature peptide. These peptides are dimerized into homodimers (with themselves) or heterodimers (with other TGF-β peptides) and are secreted from the cell. The TGF-β superfamily includes the TGF-β family, the activin family, the bone morphogenetic proteins (BMPs), the Vg-1 family, and other proteins, including glial-derived neurotrophic factor (GDNF, necessary for kidney and enteric neuron differentiation) and MUllerian inhibitory factor, which is involved in mammalian sex determination. TGF-β family members TGF-β1, 2, 3, and 5 are important in regulating the formation of the extracellular matrix between cells and for regulating cell division (both positively and negatively). TGF-β1 increases the amount of extracellular matrix epithelial cells make both by stimulating collagen and fibronectin synthesis and by inhibiting matrix degradation. TGF-βs may be critical in controlling where and when epithelia can branch to form the ducts of kidneys, lungs, and salivary glands.

Vascular Endothelial Growth Factor (VEGF). VEGFs are growth factors that mediate numerous functions of endothelial cells including proliferation, migration, invasion, survival, and permeability. The VEGFs and their corresponding receptors are key regulators in a cascade of molecular and cellular events that ultimately lead to the development of the vascular system, either by vasculogenesis, angiogenesis, or in the formation of the lymphatic vascular system. VEGF is a critical regulator in physiological angiogenesis and also plays a significant role in skeletal growth and repair.

VEGF's normal function creates new blood vessels during embryonic development, after injury, and to bypass blocked vessels. In the mature established vasculature, the endothelium plays an important role in the maintenance of homeostasis of the surrounding tissue by providing the communicative network to neighboring tissues to respond to requirements as needed. Furthermore, the vasculature provides growth factors, hormones, cytokines, chemokines and metabolites, and the like, needed by the surrounding tissue and acts as a barrier to limit the movement of molecules and cells.

The term “health-related quality of life (HRQOL)” is an individual's or a group's perceived physical and mental health over time.

The term “healthy control” as used herein refers to a subject in a state of physical well-being without signs or symptoms of a lung injury.

The term “inflammasome” as used herein refers to a multiprotein intracellular complex that detects pathogenic microorganisms and sterile stressors, and that activates the highly pro-inflammatory cytokines interleukin-1b (IL-1b) and IL-18. Inflammasomes also induce a form of cell death termed pyroptosis. Dysregulation of inflammasomes is associated with a number of autoinflammatory syndromes and autoimmune diseases. During canonical inflammasome signaling, caspase-1 cleaves pro-IL-1β to the 17 kDa bioactive cytokine, and cleaves the 52 kDa pro-GSDMD to 31 kDa N-GSDMD products, which oligomerize at the macrophage plasma membrane to generate pores that function as direct conduits for IL-1β efflux and mediators of pyroptosis. In contrast, although N-GSDMD is required for IL-1β secretion in NLRP3-activated human and murine neutrophils, N-GSDMD does not localize to the PM or increase PM permeability or pyroptosis. Instead, biochemical and microscopy studies reveal that N-GSDMD in neutrophils predominantly associates with azurophilic granules and LC3+ autophagosomes. N-GSDMD trafficking to azurophilic granules causes leakage of neutrophil elastase into the cytosol, resulting in secondary cleavage of GSDMD to an alternatively cleaved N-GSDMD product. Genetic analyses using ATG7-deficient cells indicate that neutrophils secrete IL-1β via an autophagy-dependent mechanism [Karmakar, M. et al. Nature Communic. (2020) 11: 2212, citing Shi, J. et al. Nature (2015) 526: 660-65]. The term “non-canonical inflammasome” as used herein refers to an alternate form of the inflammasome that is independent of caspase 1, but instead relies on caspase 11 (mice) or caspases 4 or 5 (human).

The term “inflammation” as used herein refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

The term “acute inflammation” as used herein refers to the rapid, short-lived (minutes to days), relatively uniform response to acute injury characterized by accumulations of fluid, plasma proteins, and neutrophilic leukocytes. Examples of injurious agents that cause acute inflammation include, but are not limited to, pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents.

The term “chronic inflammation” as used herein refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation, which includes the influx of lymphocytes and macrophages and fibroblast growth, may result in tissue scarring at sites of prolonged or repeated inflammatory activity.

During the inflammatory process, soluble inflammatory mediators of the inflammatory response work together with cellular components in a systemic fashion in the attempt to contain and eliminate the agents causing physical distress. The terms “inflammatory” or immuno-inflammatory” as used herein with respect to mediators refers to the molecular mediators of the inflammatory process. These soluble, diffusible molecules act both locally at the site of tissue damage and infection and at more distant sites. Some inflammatory mediators are activated by the inflammatory process, while others are synthesized and/or released from cellular sources in response to acute inflammation or by other soluble inflammatory mediators. Examples of inflammatory mediators of the inflammatory response include, but are not limited to, plasma proteases, complement, kinins, clotting and fibrinolytic proteins, lipid mediators, prostaglandins, leukotrienes, platelet-activating factor (PAF), peptides and amines, including, but not limited to, histamine, serotonin, and neuropeptides, proinflammatory cytokines, including, but not limited to, interleukin-1 (IL-1), interleukin-4 (IL-4), interleukin-6 (IL-6), interleukin-8, tumor necrosis factor (TNF), interferon-gamma (IFN-γ), and interleukin 12 (IL-12).

The term “injury” refers to damage or harm caused to the structure or function of the body of a subject caused by an agent or force, which may be physical or chemical.

The term “innate immunity” as used herein refers to the various innate resistance mechanisms that are encountered first by a pathogen, before adaptive immunity is induced. It includes anatomical barriers, antimicrobial peptides, the complement system, and macrophages and neutrophils carrying nonspecific pathogen-recognition receptors. It is present in all individuals at all times, does not increase with repeated exposure to a given pathogen and discriminates between groups of similar pathogens rather than responding to a particular pathogen.

The term “interferons” as used herein refers to several related families of cytokines originally named for their interference with viral replication. IFN-α and IFN-r3 are antiviral cytokines produced by a wide variety of cells in response to infection by a virus, and which also help healthy cells resist viral infection. They act through the same receptor, which signals through a Janus-family tyrosine kinase. Also known as the type 1 interferons. IFN-γ is a cytokine whose primary function is the activation of macrophages; it acts through a receptor different from that of the type I interferons. Interferon-λ, also called type II interferons, acts through a receptor different from that of the type I interferons.

The terms “lung function” or “pulmonary function” are used interchangeably to refer to the process of gas exchange called respiration (or breathing). In respiration, oxygen from incoming air enters the blood, and carbon dioxide, a waste gas from the metabolism, leaves the blood. A reduced lung function means that the ability of lungs to exchange gases is reduced.

The terms “lung interstitium” or “pulmonary interstitium” are used interchangeably herein to refer to an area located between the airspace epithelium and pleural mesothelium in the lung. Fibers of the matrix proteins, collagen and elastin, are the major components of the pulmonary interstitium. The primary function of these fibers is to form a mechanical scaffold that maintains structural integrity during ventilation.

The term “lymphocyte” refers to a small white blood cell formed in lymphatic tissue throughout the body and in normal adults making up about 22-28% of the total number of leukocytes in the circulating blood that plays a large role in defending the body against disease. Individual lymphocytes are specialized in that they are committed to respond to a limited set of structurally related antigens. This commitment, which exists before the first contact of the immune system with a given antigen, is expressed by the presence on the lymphocyte's surface membrane of receptors specific for determinants (epitopes) on the antigen. Each lymphocyte possesses a population of receptors, all of which have identical combining sites. One set, or clone, of lymphocytes differs from another clone in the structure of the combining region of its receptors and thus differs in the epitopes that it can recognize. Lymphocytes differ from each other not only in the specificity of their receptors, but also in their functions.

Two broad classes of lymphocytes are recognized: the B-lymphocytes (B-cells), which are precursors of antibody-secreting cells, and T-lymphocytes (T-cells),

B-Lymphocytes

B-lymphocytes are derived from hematopoietic cells of the bone marrow. A mature B-cell can be activated with an antigen that expresses epitopes that are recognized by its cell surface. The activation process may be direct, dependent on cross-linkage of membrane Ig molecules by the antigen (cross-linkage-dependent B-cell activation), or indirect, via interaction with a helper T-cell, in a process referred to as cognate help. In many physiological situations, receptor cross-linkage stimuli and cognate help synergize to yield more vigorous B-cell responses [Paul, W. E., “Chapter 1: The immune system: an introduction,” Fundamental Immunology, 4th Edition, Ed. Paul, W. E., Lippincott-Raven Publishers, Philadelphia (1999)].

Cross-linkage dependent B-cell activation requires that the antigen express multiple copies of the epitope complementary to the binding site of the cell surface receptors because each B-cell expresses Ig molecules with identical variable regions. Such a requirement is fulfilled by other antigens with repetitive epitopes, such as capsular polysaccharides of microorganisms or viral envelope proteins. Cross-linkage-dependent B-cell activation is a major protective immune response mounted against these microbes [Paul, W. E., “Chapter 1: The immune system: an introduction,” Fundamental Immunology, 4th Edition, Ed. Paul, W. E., Lippicott-Raven Publishers, Philadelphia (1999)].

Cognate help allows B-cells to mount responses against antigens that cannot cross-link receptors and, at the same time, provides costimulatory signals that rescue B cells from inactivation when they are stimulated by weak cross-linkage events. Cognate help is dependent on the binding of antigen by the B-cell's membrane immunoglobulin (Ig), the endocytosis of the antigen, and its fragmentation into peptides within the endosomal/lysosomal compartment of the cell. Some of the resultant peptides are loaded into a groove in a specialized set of cell surface proteins known as class II major histocompatibility complex (MHC) molecules. The resultant class II/peptide complexes are expressed on the cell surface and act as ligands for the antigen-specific receptors of a set of T-cells designated as CD4+ T-cells. The CD4+ T-cells bear receptors on their surface specific for the B-cell's class II/peptide complex. B-cell activation depends not only on the binding of the T cell through its T cell receptor (TCR), but this interaction also allows an activation ligand on the T-cell (CD40 ligand) to bind to its receptor on the B-cell (CD40) signaling B-cell activation. In addition, T helper cells secrete several cytokines that regulate the growth and differentiation of the stimulated B-cell by binding to cytokine receptors on the B cell [Paul, W. E., “Chapter 1: The immune system: an introduction,” Fundamental Immunology, 4th Edition, Ed. Paul, W. E., Lippicott-Raven Publishers, Philadelphia (1999)].

During cognate help for antibody production, the CD40 ligand is transiently expressed on activated CD4+T helper cells, and it binds to CD40 on the antigen-specific B cells, thereby transducing a second costimulatory signal. The latter signal is essential for B cell growth and differentiation and for the generation of memory B cells by preventing apoptosis of germinal center B cells that have encountered antigen. Hyperexpression of the CD40 ligand in both B and T cells is implicated in the pathogenic autoantibody production in human SLE patients [Desai-Mehta, A. et al., “Hyperexpression of CD40 ligand by B and T cells in human lupus and its role in pathogenic autoantibody production,” J. Clin. Invest. (1996), 97(9): 2063-2073].

T-Lymphocytes

T-lymphocytes derive from precursors in hematopoietic tissue, undergo differentiation in the thymus, and are then seeded to peripheral lymphoid tissue and to the recirculating pool of lymphocytes. T-lymphocytes or T cells mediate a wide range of immunologic functions. These include the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on their expression of specific cell surface molecules and the secretion of cytokines [Paul, W. E., “Chapter 1: The immune system: an introduction,” Fundamental Immunology, 4th Edition, Ed. Paul, W. E., Lippincott-Raven Publishers, Philadelphia (1999)].

T cells differ from B cells in their mechanism of antigen recognition. Immunoglobulin, the B cell's receptor, binds to individual epitopes on soluble molecules or on particulate surfaces. B-cell receptors see epitopes expressed on the surface of native molecules. Antibody and B-cell receptors evolved to bind to and to protect against microorganisms in extracellular fluids. In contrast, T cells recognize antigens on the surface of other cells and mediate their functions by interacting with, and altering, the behavior of these antigen-presenting cells (APCs). There are three main types of antigen-presenting cells in peripheral lymphoid organs that can activate T cells: dendritic cells, macrophages and B cells. The most potent of these are the dendritic cells, whose only function is to present foreign antigens to T cells. Immature dendritic cells are located in tissues throughout the body, including the skin, gut, and respiratory tract. When they encounter invading microbes at these sites, they endocytose the pathogens and their products, and carry them via the lymph to local lymph nodes or gut associated lymphoid organs. The encounter with a pathogen induces the dendritic cell to mature from an antigen-capturing cell to an antigen-presenting cell (APC) that can activate T cells. APCs display three types of protein molecules on their surface that have a role in activating a T cell to become an effector cell: (1) MHC proteins, which present foreign antigen to the T cell receptor; (2) costimulatory proteins which bind to complementary receptors on the T cell surface; and (3) cell-cell adhesion molecules, which enable a T cell to bind to the antigen-presenting cell (APC) for long enough to become activated [“Chapter 24: The adaptive immune system,” Molecular Biology of the Cell, Alberts, B. et al., Garland Science, N Y, 2002].

T-cells are subdivided into two distinct classes based on the cell surface receptors they express. The majority of T cells express T cell receptors (TCR) consisting of a and 13 chains. A small group of T cells express receptors made of γ and δ chains. Among the α/β T cells are two important sublineages: those that express the coreceptor molecule CD4 (CD4+ T cells); and those that express CD8 (CD8+ T cells). These cells differ in how they recognize antigen and in their effector and regulatory functions.

CD4+ T cells are the major regulatory cells of the immune system. Their regulatory function depends both on the expression of their cell-surface molecules, such as CD40 ligand whose expression is induced when the T cells are activated, and the wide array of cytokines they secrete when activated.

T cells also mediate important effector functions, some of which are determined by the patterns of cytokines they secrete. The cytokines can be directly toxic to target cells and can mobilize potent inflammatory mechanisms.

In addition, T cells particularly CD8+ T cells, can develop into cytotoxic T-lymphocytes (CTLs) capable of efficiently lysing target cells that express antigens recognized by the CTLs [Paul, W. E., “Chapter 1: The immune system: an introduction,” Fundamental Immunology, 4th Edition, Ed. Paul, W. E., Lippincott-Raven Publishers, Philadelphia (1999)].

T cell receptors (TCRs) recognize a complex consisting of a peptide derived by proteolysis of the antigen bound to a specialized groove of a class II or class I MHC protein. The CD4+ T cells recognize only peptide/class II complexes while the CD8+ T cells recognize peptide/class I complexes [Paul, W. E., “Chapter 1: The immune system: an introduction,” Fundamental Immunology, 4th Edition, Ed. Paul, W. E., Lippincott-Raven Publishers, Philadelphia (1999)].

The TCR's ligand (i.e., the peptide/MHC protein complex) is created within antigen-presenting cells (APCs). In general, class II MHC molecules bind peptides derived from proteins that have been taken up by the APC through an endocytic process. These peptide-loaded class II molecules are then expressed on the surface of the cell, where they are available to be bound by CD4+ T cells with TCRs capable of recognizing the expressed cell surface complex. Thus, CD4+ T cells are specialized to react with antigens derived from extracellular sources [Paul, W. E., “Chapter 1: The immune system: an introduction,” Fundamental Immunology, 4th Edition, Ed. Paul, W. E., Lippincott-Raven Publishers, Philadelphia (1999)]. Stimulation of the MHC II pathway leads to induction of a wide range of adaptive immune responses, including activation of macrophages and activation of B cells to secrete antibodies, as well as activation of cytotoxic T cells to kill targeted cells.

In contrast, class I MHC molecules are mainly loaded with peptides derived from internally synthesized proteins, such as viral proteins. These peptides are produced from cytosolic proteins by proteolysis by the proteasome and are translocated into the rough endoplasmic reticulum. Such peptides, generally nine amino acids in length, are bound into the class I MHC molecules and are brought to the cell surface, where they can be recognized by CD8+ T cells expressing appropriate receptors. This gives the T cell system, particularly CD8+ T cells, the ability to detect cells expressing proteins that are different from, or produced in much larger amounts than, those of cells of the remainder of the organism (e.g., viral antigens) or mutant antigens (such as active oncogene products), even if these proteins in their intact form are neither expressed on the cell surface nor secreted [Paul, W. E., “Chapter 1: The immune system: an introduction,” Fundamental Immunology, 4th Edition, Ed. Paul, W. E., Lippincott-Raven Publishers, Philadelphia (1999)]. Activation of the MHC I pathway leads to induction of cytotoxic CD8+ T cells only.

T cells can also be classified based on their function as helper T cells; T cells involved in inducing cellular immunity; suppressor T cells; and cytotoxic T cells.

Helper T Cells

Helper T cells are T cells that stimulate B cells to make antibody responses to proteins and other T cell-dependent antigens. T cell-dependent antigens are immunogens in which individual epitopes appear only once or a limited number of times such that they are unable to cross-link the membrane immunoglobulin (Ig) of B cells or do so inefficiently. B cells bind the antigen through their membrane Ig, and the complex undergoes endocytosis. Within the endosomal and lysosomal compartments, the antigen is fragmented into peptides by proteolytic enzymes and one or more of the generated peptides are loaded into class II MHC molecules, which traffic through this vesicular compartment. The resulting peptide/class II MHC complex is then exported to the B-cell surface membrane. T cells with receptors specific for the peptide/class II molecular complex recognize this complex on the B-cell surface [Paul, W. E., “Chapter 1: The immune system: an introduction,” Fundamental Immunology, 4th Edition, Ed. Paul, W. E., Lippincott-Raven Publishers, Philadelphia (1999)].

B-cell activation depends both on the binding of the T cell through its TCR and on the interaction of the T-cell CD40 ligand (CD40L) with CD40 on the B cell. T cells do not constitutively express CD40L. Rather, CD40L expression is induced as a result of an interaction with an APC that expresses both a cognate antigen recognized by the TCR of the T cell and CD80 or CD86. CD80/CD86 is generally expressed by activated, but not resting, B cells so that the helper interaction involving an activated B cell and a T cell can lead to efficient antibody production. In many cases, however, the initial induction of CD40L on T cells is dependent on their recognition of antigen on the surface of APCs that constitutively express CD80/86, such as dendritic cells. Such activated helper T cells can then efficiently interact with and help B cells. Cross-linkage of membrane Ig on the B cell, even if inefficient, may synergize with the CD40L/CD40 interaction to yield vigorous B-cell activation. The subsequent events in the B-cell response, including proliferation, Ig secretion, and class switching (of the Ig class being expressed) either depend or are enhanced by the actions of T cell-derived cytokines [Paul, W. E., “Chapter 1: The immune system: an introduction,” Fundamental Immunology, 4th Edition, Ed. Paul, W. E., Lippincott-Raven Publishers, Philadelphia (1999)].

CD4+ T cells tend to differentiate into cells that principally secrete the cytokines IL-4, IL-5, IL-6, and IL-10 (TH2 cells) or into cells that mainly produce IL-2, IFN-γ, and lymphotoxin (TH1 cells). The TH2 cells are very effective in helping B-cells develop into antibody-producing cells, whereas the TH1 cells are effective inducers of cellular immune responses, involving enhancement of microbicidal activity of monocytes and macrophages, and consequent increased efficiency in lysing microorganisms in intracellular vesicular compartments. Although the CD4+ T cells with the phenotype of TH2 cells (i.e., IL-4, IL-5, IL-6 and IL-10) are efficient helper cells, TH1 cells also have the capacity to be helpers [Paul, W. E., “Chapter 1: The immune system: an introduction,” Fundamental Immunology, 4th Edition, Ed. Paul, W. E., Lippincott-Raven Publishers, Philadelphia (1999)].

T Cells Involved in Induction of Cellular Immunity

T cells also may act to enhance the capacity of monocytes and macrophages to destroy intracellular microorganisms. In particular, interferon-gamma (IFN-γ) produced by helper T cells enhances several mechanisms through which mononuclear phagocytes destroy intracellular bacteria and parasitism including the generation of nitric oxide and induction of tumor necrosis factor (TNF) production. The TH1 cells are effective in enhancing the microbicidal action because they produce IFN-γ. By contrast, two of the major cytokines produced by TH2 cells, IL-4 and IL-10, block these activities. [Paul, W. E., “Chapter 1: The immune system: an introduction,” Fundamental Immunology, 4th Edition, Ed. Paul, W. E., Lippincott-Raven Publishers, Philadelphia (1999)].

Suppressor or Regulatory T (Treg) Cells

A controlled balance between initiation and downregulation of the immune response is important to maintain immune homeostasis. Both apoptosis and T cell anergy (a tolerance mechanism in which the T cells are intrinsically functionally inactivated following an antigen encounter [Schwartz, R. H., “T cell anergy,” Annu. Rev. Immunol. (2003) 21: 305-334] are important mechanisms that contribute to the downregulation of the immune response. A third mechanism is provided by active suppression of activated T cells by suppressor or regulatory CD4+T (Treg) cells [Reviewed in Kronenberg, M. et al., “Regulation of immunity by self-reactive T cells,” Nature 435: 598-604 (2005)]. CD4+ Tregs that constitutively express the IL-2 receptor alpha (IL-2Ra) chain (CD4+CD25+) are a naturally occurring T cell subset that are anergic and suppressive [Taams, L. S. et al., “Human anergic/suppressive CD4+CD25+ T cells: a highly differentiated and apoptosis-prone population,” Eur. J. Immunol., 31: 1122-1131 (2001)]. Depletion of CD4+CD25+ Tregs results in systemic autoimmune disease in mice. Furthermore, transfer of these Tregs prevents development of autoimmune disease. Human CD4+CD25+ Tregs, similar to their murine counterpart, are generated in the thymus and are characterized by the ability to suppress proliferation of responder T cells through a cell-cell contact-dependent mechanism, the inability to produce IL-2, and the anergic phenotype in vitro. Human CD4+CD25+ T cells can be split into suppressive (CD25^{high}) and nonsuppressive (CD25^{low}) cells, according to the level of CD25 expression. A member of the forkhead family of transcription factors, FOXP3, has been shown to be expressed in murine and human CD4+CD25+ Tregs and appears to be a master gene controlling CD4+CD25+ Treg development [Battaglia, M. et al., “Rapamycin promotes expansion of functional CD4+CD25+Foxp3+ regulator T cells of both healthy subjects and type 1 diabetic patients,” J. Immunol., 177: 8338-8347 (200)].

Cytotoxic T Lymphocytes (CTL)

The CD8+ T cells that recognize peptides from proteins produced within the target cell have cytotoxic properties in that they lead to lysis of the target cells. The mechanism of CTL-induced lysis involves the production by the CTL of perforin, a molecule that can insert into the membrane of target cells and promote the lysis of that cell. Perforin-mediated lysis is enhanced by a series of enzymes produced by activated CTLs, referred to as granzymes. Many active CTLs also express large amounts of fas ligand on their surface. The interaction of fas ligand on the surface of CTL with fas on the surface of the target cell initiates apoptosis in the target cell, leading to the death of these cells. CTL-mediated lysis appears to be a major mechanism for the destruction of virally infected cells.

The term “modulate” as used herein means to regulate, alter, adapt, or adjust to a certain measure or proportion.

The term “natural killer (NK) cells” as used herein is meant to refer to lymphocytes in the same family as T and B cells, classified as group I innate lymphocytes. NK cells have an ability to kill invading pathogens cells without any priming or prior activation, in contrast to cytotoxic T cells, which need priming by antigen presenting cells. NK cells secrete cytokines such as IFNγ and TNFα, which act on other immune cells, like macrophages and dendritic cells, to enhance the immune response. Activating receptors on the NK cell surface recognize molecules expressed on the surface of cancer cells and infected cells and switch on the NK cell. Inhibitory receptors act as a check on NK cell killing. Most normal healthy cells express MHCI receptors, which mark them as “self” Inhibitory receptors on the surface of the NK cell recognize cognate MHCI, which switches off the NK cell, preventing it from killing. Once the decision is made to kill, the NK cell releases cytotoxic granules containing perforin and granzymes, which leads to lysis of the target cell. Natural killer reactivity, including cytokine secretion and cytotoxicity, is controlled by a balance of several germ-line encoded inhibitory and activating receptors such as killer immunoglobulin-like receptors (KIRs) and natural cytotoxicity receptors (NCRs). The presence of the MHC Class I molecule on target cells serves as one such inhibitory ligand for MHC Class I-specific receptors, the Killer cell Immunoglobulin-like Receptor (KIR), on NK cells. Engagement of KIR receptors blocks NK activation and, paradoxically, preserves their ability to respond to successive encounters by triggering inactivating signals. Therefore, if a KIR is able to sufficiently bind to MHC Class I, this engagement may override the signal for killing and allows the target cell to live. In contrast, if the NK cell is unable to sufficiently bind to MHC Class I on the target cell, killing of the target cell may proceed.

The term “neutrophil” as used herein refers to a phagocytic white blood cell in human peripheral blood, with a multilobed nucleus and granules that stain with neutral stains. They enter infected tissues and engulf and kill extracellular pathogens.

The term “neutophil elastase” as used herein refers to a proteolytic enzyme stored in the granules of neutrophils involved in the processing of antimicrobial peptides.

The abbreviation “NFκB” as used herein refers to which is a proinflammatory transcription factor. It switches on multiple inflammatory genes, including cytokines, chemokines, proteases, and inhibitors of apoptosis, resulting in amplification of the inflammatory response [Barnes, P J, (2016) Pharmacol. Rev. 68: 788-815]. The molecular pathways involved in NF-κB activation include several kinases. The classic (canonical) pathway for inflammatory stimuli and infections to activate NF-κB signaling involve the IKK (inhibitor of KB kinase) complex, which is composed of two catalytic subunits, IKK-α and IKK-β, and a regulatory subunit IKK-γ (or NFκB essential modulator [Id., citing Hayden, M S and Ghosh, S (2012) Genes Dev. 26: 203-234]. The IKK complex phosphorylates Nf-κB-bound IKBs, targeting them for degradation by the proteasome and thereby releasing NF-κB dimers that are composed of p65 and p50 subunits, which translocate to the nucleus where they bind to KB recognition sites in the promoter regions of inflammatory and immune genes, resulting in their transcriptional activation. This response depends mainly on the catalytic subunit IKK-β (also known as IKK2), which carries out IκB phosphorylation. The noncanonical (alternative) pathway involves the upstream kinase NF-κB-inducing kinase (NIK) that phosphorylates IKK-α homodimers and releases RelB and processes p100 to p52 in response to certain members of the TNF family, such as lymphotoxin-β [Id., citing Sun, S C. (2012) Immunol. Rev. 246: 125-140]. This pathway switches on different gene sets and may mediate different immune functions from the canonical pathway. Dominant-negative IKK-β inhibits most of the proinflammatory functions of NF-κB, whereas inhibiting IKK-α has a role only in response to limited stimuli and in certain cells such as B-lymphocytes. The noncanonical pathway is involved in development of the immune system and in adaptive immune responses. The coactivator molecule CD40, which is expressed on antigen-presenting cells, such as dendritic cells and macrophages, activates the noncanonical pathway when it interacts with CD40L expressed on lymphocytes [Id., citing Lombardi, V et al. (2010) Int. Arch. Allergy Immunol. 151: 179-89].

The term “NOD-like receptors (NLRs)” as used herein refers to a large family of proteins containing a nucleotide-oligomerization domain (NOD) associated with various other domains, and whose general function is the detection of microbes and of cellular stress. NOD1 and NOD2 are intracellular proteins of the NOD subfamily that contain a leucine-rich repeat (LRR) domain that binds components of bacterial cell walls to active the NFκB pathway and initiate inflammatory responses.

The term “NLRP3”, sometimes called NALP3″, as used herein refers to a member of the family of intracellular NOD-like receptor proteins that have pyrin domains. It acts as a sensor of cellular damage and is part of the inflammasome.

The term “overall survival” as used herein refers to the length of time from either the date of diagnosis or the start of treatment for a disease that patients diagnosed with the disease are still alive.

The term “paracrine signaling” as used herein refers to delivery of a local mediator of cell communication over a short distance by a local mediator of cell communication.

The term “parenteral” as used herein refers to a route of administration where the drug or agent enters the body without going through the stomach or “gut”, and thus does not encounter the first pass effect of the liver. Examples include, without limitation, introduction into the body by way of an injection (i.e., administration by injection), including, for example, subcutaneously (i.e., an injection beneath the skin), intramuscularly (i.e., an injection into a muscle); intravenously (i.e., an injection into a vein), intrathecally (i.e., an injection into the space around the spinal cord or under the arachnoid membrane of the brain), or infusion techniques.

The term “perfusion” as used herein refers to the process of nutritive delivery of arterial blood to a capillary bed in biological tissue. Perfusion (“F”) can be calculated with the formula F=(P_{A}−Pᵥ)/R wherein P_{A} is mean arterial pressure, Pᵥ is mean venous pressure, and R is vascular resistance. Tissue perfusion can be measured in vivo, by, for example, but not limited to, magnetic resonance imaging (MRI) techniques. Such techniques include using an injected contrast agent and arterial spin labeling (ASL), wherein arterial blood is magnetically tagged before it enters into the tissue of interest and the amount of labeling is measured and compared to a control recording.

The term “peripheral blood mononuclear cell” or “PBMC” as used herein refers to a type of white blood cell that contains one nucleus, such as a lymphocyte or a macrophage.

The term “pharmaceutical composition” is used herein to refer to a composition that is employed to prevent, reduce in intensity, cure or otherwise treat a target condition or disease.

As used herein the phrase “pharmaceutically acceptable carrier” refers to any substantially non-toxic carrier useable for formulation and administration of the composition of the described invention in which the product of the described invention will remain stable and bioavailable. The pharmaceutically acceptable carrier must be of sufficiently high purity and of sufficiently low toxicity to render it suitable for administration to the mammal being treated. It further should maintain the stability and bioavailability of an active agent. The pharmaceutically acceptable carrier can be liquid or solid and is selected, with the planned manner of administration in mind, to provide for the desired bulk, consistency, etc., when combined with an active agent and other components of a given composition.

The term “pharmacologic effect”, as used herein, refers to a result or consequence of exposure to an active agent.

The term “pneumocytes” as used herein refers to surface epithelial cells of the alveoli, of which there are two types. The type I pneumocytes form part of the barrier across which gas exchange occurs. They can be identified as thin, squamous cells whose most obvious feature is their nuclei. Type II pneumocytes are larger, cuboidal cells and occur more diffusely than type I cells. They appear foamier than type I cells because they contain phospholipid multilamellar bodies, the precursor to pulmonary surfactant. Capillaries form a plexus around each alveolus.

As used herein, the term “potent” or “potency” refers to the necessary biological activity of the CLBS119 CD34+ cell product of the described invention, i.e., potent cells of the described invention remain viable, are capable of mediated mobility, and are able to grow, i.e., to form hematopoietic colonies in an in vitro CFU assay.

The term “progenitor cell” as used herein refers to an early descendant of a stem cell that can only differentiate, but can no longer renew itself. Progenitor cells mature into precursor cells that mature into mature phenotypes. Hematopoietic progenitor cells are referred to as colony-forming units (CFU) or colony-forming cells (CFC). The specific lineage of a progenitor cell is indicated by a suffix, such as, but not limited to, CFU-E (erythrocytic), CFU-F (fibroblastic), CFU-GM (granulocytic/macrophage), and CFU-GEMM (pluripotent hematopoietic progenitor).

The term “progression” as used herein refers to the course of a disease as it becomes worse or spreads in the body.

The term “progression-free survival” as used herein refers to the length of time during and after treatment in which a patient is living with a disease that does not get worse.

The term “pulmonary compliance” as used herein refers to the change in lung volume per unit change in pressure. “Dynamic compliance” is the volume change divided by the peak inspiratory transthoracic pressure. “Static compliance” is the volume change divided by the plateau inspiratory pressure. Pulmonary compliance measurements reflect the elastic properties of the lungs and thorax and are influenced by factors such as degree of muscular tension, degree of interstitial lung water, degree of pulmonary fibrosis, degree of lung inflation, and alveolar surface tension [Doyle D J, O'Grady K F. Physics and Modeling of the Airway, D, in Benumof and Hagberg's Airway Management, 2013]. Total respiratory system compliance is given by the following formula: C=ΔV/ΔP, where ΔV=change in lung volume, and ΔP=change in airway pressure. This total compliance may be related to lung compliance and thoracic (chest wall) compliance by the following relation: 1/C_{T}=1/C_{L}+1/C_{Th}, where C_{T}=total compliance (e.g., 100 mL/cm H₂O); C_{L}=lung compliance (e.g., 200 mL/cm H₂O), and C_{Th}=thoracic compliance (e.g., 200 mL/cm H₂O). The values shown in parentheses are some typical normal adult values that can be used for modeling purposes [Id].

The term “pulmonary vascular endothelium” as used herein refers to the monolayer of cells that lines all vessels. It is a multidimensional tissue whose specialized functions include direct lung vascular barrier regulation, participation in the initiation and resolution of inflammatory responses and the processing of mediators before delivery to the systemic circulation.

The term “purification” and its various grammatical forms as used herein refers to the process of isolating or freeing from foreign, extraneous, or objectionable elements. Because compositions may be admixed with a pharmaceutically-acceptable carrier in a pharmaceutical preparation, the compositions may comprise only a small percentage by weight of the preparation. The composition is nonetheless substantially pure in that it has been substantially separated from the substances with which it may be associated in living systems or during synthesis. Exemplary analytical protocols that can be used to determine purity include, without limitation, FACS, HPLC, gel electrophoresis, chromatography, and the like.

The term “pyroptosis” as used herein refers to a pro-inflammatory mode of lytic cell death mediated by Gasdermin D (GSDMD) [Karmakar, M. et al. Nature Communic. (2020) 11: 2212, citing Shi, J. et al. Nature (2015) 526: 660-65]. The Gasdermin (GSDM) family of proteins are regulators of innate immune and cell death responses. GSDMs are expressed as ˜50 kDa cytosolic pro-proteins with N-terminal effector and C-terminal regulatory domains, and a binding interface between the C-terminal domain and the ˜30 kDa N-GSDM effector moiety maintains pro-GSDM in an auto-inhibited conformation. Disruption of this interface by proteolytic cleavage of linker loops or mutation of key residues induces conformational rearrangement of N-GSDM subunits [Id., citing Broz, P. et al. Nat. Rev. Immunol. (2019) doi.org/10.1038/s41577-019-0228-2; Sjo. K., et al. Trends Biochem. Sci. (2017) 42: 245-54; Kovacs, S. B. Miao, E A. Trends Cell Biol. (2017) 27: 673-84] to expose sites for interaction with anionic phospholipids on accessible leaflets of membrane bilayers. This facilitates N-GSDM oligomerization and drives insertion of multiple β-hairpins through the targeted bilayer to assemble macropores (10-18 nm inner diameters). Assembly of N-GSDM pores in the plasma membrane markedly increases its permeability to macromolecules (up to 20 kDa), metabolites, ions, and major osmolytes, resulting in rapid collapse of cellular integrity to facilitate pyroptosis [Id., citing Sborgi, L. EMBO J. (2016) 35: 1766-78; Liu, X. et al. Nature [2016]535: 153-58; Ding, J. ET al. Nature (2016) 535: 111-16]. In infected tissues, pyroptosis eliminates the replicative niche of intracellular bacteria within dying macrophages to entrap bacteria for ingestion by recruited neutrophils [Id., citing Jorgensen, I. et al. J. Exp. Med. (2016) 213: 2113-38].

The term “recipient” as used herein refers to one who receives.

The term “repair” as used herein as a noun refers to any correction, reinforcement, reconditioning, remedy, making sound, renewal, mending, patching, or the like that restores function. When used as a verb, it means to correct, to reinforce, to recondition, to remedy, to make sound, to renew, to mend, to patch or to otherwise restore function.

The term “restore” as used herein refers to bringing back to a normal condition; to bring back to health or strength.

The term “stem cells” refers to undifferentiated cells having high proliferative potential with the ability to self-renew that can generate daughter cells that can undergo terminal differentiation into more than one distinct cell phenotype. The term “renewal” or “self renewal” as used herein, refers to the process by which a stem cell divides to generate one (asymmetric division) or two (symmetric division) daughter cells having development potential indistinguishable from the mother cell. Self renewal involves both proliferation and the maintenance of an undifferentiated state. The term “adult (somatic) stem cells” as used herein refers to undifferentiated cells found among differentiated cells in a tissue or organ. Their primary role in vivo is to maintain and repair the tissue in which they are found. Adult stem cells, which have been identified in many organs and tissues, including brain, bone marrow, peripheral blood, blood vessels, skeletal muscles, skin, teeth, gastrointestinal tract, liver, ovarian epithelium, and testis, are thought to reside in a specific area of each tissue, known as a stem cell niche, where they may remain quiescent (non-dividing) for long periods of time until they are activated by a normal need for more cells to maintain tissue, or by disease or tissue injury. Mesenchymal stem cells are an example of adult stem cells.

The term “stem cell mobilization” as used herein refers to a process whereby stem cells are stimulated by certain drugs to cause movement of the stem cells from the bone marrow space into the bloodstream so they are available for collection and storage. Stem cell mobilization can be induced by a wide variety of “mobilizing” agents, including, but not limited to, antagonists of adhesion and chemotaxis, cytotoxic drugs, and certain cytokines, which often drive both hematopoietic stem cell (HSC) proliferation and movement from the marrow to the bloodstream (e.g., G-CSF, GM-CSF, IL-7, IL-3, IL-12, Stem cell factor (SCF), and flt-3 ligand; chemokines like IL-8, Mip-1α, Groβ, or SDF-1; and chemotherapeutic agents cyclophosphamide and paclitaxel [Id., citing T. Lapidot and I. Petit, Exptl Hematology 30: 973-981 (2002) at 974].

The term “stem cell trafficking/migration” refers to the oriented or directed movement of a cell towards a particular anatomic destination. There are two principal modes of stem cell trafficking: stem cell homing and interstitial migration. The term “stem cell homing” as used herein refers to a process whereby stem cells are disseminated throughout the body by the flowing blood until they recognize and interact with microvascular endothelial cells in a particular target organ, it always is preceded and followed by an active migratory phase during which cells must navigate the extravascular compartment to access the blood from their point of origin and to reach their final destination in a distant target organ. Trafficking/migration via homing appears to comprise three consecutive steps that rely on distinct receptor-ligand pathways: (1) tethering and rolling, mediated by primary adhesion molecules (selectins or α4-integrins) with fast binding kinetics and high tensile strength but short bond lifetime; (2) a chemotactic/activating stimulus provided by soluble or surface-bound chemoattractants, which signal mostly through Gαi-coupled seven transmembrane domain receptors; and (3) sticking, mediated by secondary adhesion molecules, mostly integrins (132 or α4) that interact with endothelial ligands of the immunoglobulin superfamily (IgSF). The term “stem cell interstitial migration” as used herein refers to a process that stem cells recognize and obey extravascular guidance cues. It requires active ameboid movement and can occur independent of blood flow [Laird, Diana J. Cell 132: 612-30 (20008) at 612-13].

The term “Stromal cell-Derived Factor-1” (“SDF-1”) (also designated as CXCL12) is a homeostatic chemokine that signals through its main receptor CXCR-4. Chemotactic signaling via the SDF-1α/CXCR-4 axis is a broadly conserved migration mechanism that acts in stem cell movements in multiple tissues in both the embryo and the adult [Diana J. Laird, et al., Cell 132: 612-630 (2008) at 624-26]. During development, SDF-1α/CXCR-4 signals direct the homing of fetal mouse HSCs to the liver and marrow and help to target mouse myogenic precursor cells [Id]. Immature CXCR4null progenitor cells (i.e., c-kit+ Sca−1+Lin−/low cells with a stem cell phenotype) recovered from murine fetal liver do not qualify as hematopoietic stem cells: they do not migrate to a gradient of SDF-1 in vitro; they are unable to home and repopulate the bone marrow of the developing embryo; and they fail to give rise to high levels of multilineage myeloid and lymphoid cells in the bone marrow and peripheral blood of primary and serially transplanted secondary murine recipients, which is essential for a repopulating cell in order to qualify as a pluripotent stem cell with self-renewal potential [Id., citing T. Lapidot and I. Petit, Exptl Hematology 30: 973-981 (2002) at 976]. In the adult, SDF-la and CXCR-4 are implicated in the mobilization of mouse and human HSCs into the peripheral blood and their reentry into the marrow; skeletal muscle regeneration; the dissemination of tumor-forming cells in a large number of metastatic cancers; in survival/antiapoptosis of HSCs/HPCs; and regulate several processes apparently unrelated to stem cell activity, including the normal trafficking of lymphocyte precursors and mature hematopoietic cells, migration of cerebellar neurons, and cardiogenesis [Diana J. Laird, et al., Cell 132: 612-630 (2008) at 624-26; citing Broxmeyer, H. E., et al., J. Exp. Med. 201(8): 1307-18 (2005), at 1308]. The CXCR-4-SDF-1 (CXCL-12) axis also plays a role in physiologic tissue repair and regeneration. [Burger and T. J. Kipps, Blood (2006). 107: 1761-67]. Physiologic repair of ischemic injuries involves the selective recruitment of circulating or resident progenitor cells. Hypoxia-inducible factor 1 (HIF-1), a central mediator of tissue hypoxia, induces SDF-1 expression in ischemic areas in direct proportion to reduced oxygen tension in vivo. HIF-1-induced SDF-1 expression on endothelial cells attracts circulating CXCR-4-expressing stem and progenitor cells, to areas of tissue damage. As such, hypoxia induces a transient, conditional stem cell niche for recruitment of these CXCR-4 mediated progenitor cells for tissue repair. The expression of SDF-1 normalizes after regular oxygen tension has been restored during tissue regeneration. In addition to inducing SDF-1, HIF-1 enhances the expression and function of CXCR-4 [Burger and T. J. Kipps, Blood (2006). 107: 1761-67]. In addition to its fundamental role in recruiting CXCR-4+ cells at the site of neo-angiogenesis, SDF-1 has important functions in inducing, controlling and regulating vascularization of tumors and damaged tissues. It directly participates in new blood vessel formation: SDF-1 has an angiogenic effect on endothelial cells by inducing cell proliferation, differentiation, sprouting and tube formation in vitro and by preventing the apoptosis of EPCs [Petit, I. et al. Trends Immunol. (2007) 28 (7): 299-307, citing Salvucci, O. et al. Blood (2002) 99: 2703-11; Yamaguchi, J. et al. Circulation (2003) 107: 1322-28]. In vivo, SDF-1 placed in matrigel plugs induces angiogenesis [Id., citing Buckingham, M. et al. J. Anat. (2003) 202: 59-68]. SDF exerts a more potent pro-angiogenic effect when delivered in combination with VEGF-A [Id., citing Kryczek, I., et al. Cancer Res. 92005) 65: 465-72; Can, A N et al. Cardiovaswc. Res. (2006) 69: 925-35]. SDF-1 also modulates vascularization of ischemic tissues and tumors by influencing the expression of other angiogenic factors [Id., citing Wang, J. et al. Cell Signal (2005) 17: 1578-92]. SDF-1 also decreases production of the anti-angiogenic molecule angiostatin [Id., citing Wang, J. et al. Cell Signal (2005) 17: 1578-92, Wang, J. et al. Cancer Res. (2007) 67: 149-59]. In addition, SDF-1 induces the production of metalloproteinases, enzymes essential to deploying angiogenic factors, thereby accelerating tissue remodeling during vascularization [Id., citing Grunewald, M., et al. Cell (2006) 124: 175-89; Heissig, B. et al. Curr Opin. Hematol. (2003) 10: 136-41; Petit, I. et al. J. Cell Invest. (2005) 115: 168-76]. Lastly, SDF-1 contributes to the stabilization of neo-vessel formation by recruiting CXCR-4+ PDGFR+ckit+ smooth muscle progenitors during recovery from vascular injury [Id., citing Zernecke, A. et al. Cir. Res. (2005) 96: 784-91].

The terms “subject” or “individual” or “patient” are used interchangeably to refer to a member of an animal species of mammalian origin, including humans.

The term “subject at risk of lung injury” is a subject who has one or more predisposing factors to the development of lung injury following a severe virus infection Examples of such predisposing factors include, without limitation, the very young, the elderly, those with pre-existing health conditions, such as chronic cardiopulmonary or renal disease; diabetes, immunosuppression, or severe anemia, those who are ill; and those who are physically weak, e.g., due to malnutrition or dehydration.

The terms “surfactant protein A (SP-A)” and “surfactant protein D (SP-D)” refer to hydrophobic, collagen-containing calcium-dependent lectins, with a range of nonspecific immune functions at pulmonary and cardiopulmonary sites. SP-A and SP-D play crucial roles in the pulmonary immune response, and are secreted by type II pneumocytes, nonciliated bronchiolar cells, submucosal glands, and epithelial cells of other respiratory tissues, including the trachea and bronchi. SP-D is important in maintaining pulmonary surface tension, and is involved in the organization, stability, and metabolism of lung parenchyma [Wang K, et al. Medicine (2017) 96 (23): e7083]. An increase of 49 ng/mL (1 SD) in baseline SP-A level was associated with a 3.3-fold increased risk of mortality in the first year after presentation. SP-A and SP-D are predictors of worse survival in a one year mortality regression model [Guiot, J. et al. Lung (2017) 195(3): 273-280].

The term “symptom” as used herein refers to a sign or an indication of disorder or disease, especially when experienced by an individual as a change from normal function, sensation, or appearance.

The term “T cell exhaustion” as used herein refers to a state of T cell dysfunction that arises during many chronic infections and cancer. It is defined by poor effector function, sustained expression of inhibitory receptors and a transcriptional state distinct from that of functional effector or memory T cells. Modulating pathways overexpressed in exhaustion—for example, by targeting programmed cell death protein 1 (PD1) and cytotoxic T lymphocyte antigen 4 (CTLA4)—can reverse this dysfunctional state and reinvigorate immune responses [Wherry E J and Kurachi, M. Nature (2015) 15: 486-99, citing Wherry E J. Nat. Immunol. (2011) 131:492-499; Schietinger A, Greenberg P D. Trends Immunol. (2014) 35:51-60; Barber D L, et al. Restoring function in exhausted CD8 T cells during chronic viral infection. Nature. (2006) 439:682-687; Nguyen L T, Ohashi P S. Nat. Rev. Immunol. (2014) 15:45-56]. The level and duration of chronic antigen stimulation and infection seem to be key factors that lead to T cell exhaustion and correlate with the severity of dysfunction during chronic infection. Examples of inhibitory receptors include the inhibitory pathways mediated by PD1 in response to binding of PD1 ligand 1 (PDL1) and/or PDL2 [Id., citing Okazaki T, et al., Nature Immunol. (2013) 14:1212-1218, Odorizzi P M, Wherry E J. J. Immunol. (2012) 188:2957-2965, Araki K, et al. Cold Spring Harb. Symp. Quant. Biol. (2013) 78:239-247]. Exhausted T cells can co-express PD1 together with lymphocyte activation gene 3 protein (LAG3), 2B4 (also known as CD244), CD160, T cell immunoglobulin domain and mucin domain-containing protein 3 (TIM3; also known as HAVCR2), CTLA4 and many other inhibitory receptors [Id., citing Blackburn S D, et al. Nat. Immunol. (2009) 10:29-37]. Typically, the higher the number of inhibitory receptors co-expressed by exhausted T cells, the more severe the exhaustion. It has been suggested that inhibitory receptors such as PD1 might regulate T cell function in several ways [Id., citing Schietinger A, Greenberg P D. Trends Immunol. (2014) 35:51-60; Odorizzi P M, Wherry E J. J. Immunol. (2012) 188:2957-2965], e.g., by ectodomain competition, which refers to inhibitory receptors sequestering target receptors or ligands and/or preventing the optimal formation of microclusters and lipid rafts (for example, CTLA4); second, through modulation of intracellular mediators, which can cause local and transient intracellular attenuation of positive signals from activating receptors such as the TCR and co-stimulatory receptors [Id., citing Parry R V, et al. Molec. Cell. Biol. (2005) 25:9543-9553; Yokosuka T, et al. J. Exp. Med. (2012) 209:1201-1217; Clayton K L, et al. J. Immunol. (2014) 192:782-791]; and third, through the induction of inhibitory genes [Id., citing Quigley M, et al. Nat. Med. (2010) 16:1147-1151]. Soluble molecules are a second class of signals that regulate T cell exhaustion; these include immunosuppressive cytokines such as IL-10 and transforming growth factor-0 (TGF0) and inflammatory cytokines, such as type I interferons (IFNs) and IL-6 [Id.]

The terms “therapeutic amount”, an “effective amount”, or “pharmaceutical amount” of one or more of the active agents are used interchangeably to refer to an amount that is sufficient to provide the intended benefit of treatment. Dosage levels are based on a variety of factors, including the type of injury, the age, weight, sex, medical condition of the patient, the severity of the condition, the route of administration, and the particular active agent employed. Thus the dosage regimen may vary widely, but can be determined routinely by a physician using standard methods. Additionally, the terms “therapeutic amount” and “pharmaceutical amount” include prophylactic or preventative amounts of the compositions of the described invention. In prophylactic or preventative applications of the described invention, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. For any therapeutic agent described herein the therapeutically effective amount may be initially determined from preliminary in vitro studies and/or animal models. A therapeutically effective dose may also be determined from human data. The applied dose may be adjusted based on the relative bioavailability and potency of the administered agent.

The intensity of effect of a drug (y-axis) can be plotted as a function of the dose of drug administered (X-axis). [Goodman & Gilman's The Pharmacological Basis of Therapeutics, Ed. Joel G. Hardman, Lee E. Limbird, Eds., 10th Ed., McGraw Hill, New York (2001), p. 25, 50]. These plots are referred to as dose-effect curves. Such a curve can be resolved into simpler curves for each of its components. These concentration-effect relationships can be viewed as having four characteristic variables: potency, slope, maximal efficacy, and individual variation. The location of the dose-effect curve along the concentration axis is an expression of the potency of a drug. [Id]. The slope of the dose-effect curve reflects the mechanism of action of a drug. The steepness of the curve dictates the range of doses useful for achieving a clinical effect. The term “maximal or clinical efficacy” refers to the maximal effect that can be produced by a drug. Maximal efficacy is determined principally by the properties of the drug and its receptor-effector system and is reflected in the plateau of the curve. In clinical use, a drug's dosage may be limited by undesired effects. Because of biological variability, an effect of varying intensity may occur in different individuals at a specified concentration or a drug. It follows that a range of concentrations may be required to produce an effect of specified intensity in all subjects. Lastly, different individuals may vary in the magnitude of their response to the same concentration of a drug when the appropriate correction has been made for differences in potency, maximal efficacy and slope.

The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50, which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation (meaning a perceptible, outward or visible expression of a disease or abnormal condition). A therapeutic effect also may include, directly or indirectly, the arrest reduction or elimination of the progression of a disease manifestation.

General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained.

The term “TIE2” as used herein refers to an endothelial cell specific receptor that is activated by angiopoietins, growth factors required for angiogenesis.

The term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

The term “vascular injury” refers to an injury to the vasculature (i.e., the vascular network, meaning the network of blood vessels or ducts that convey fluids, such as, without limitation, blood or lymph).

The term “vascular permeability” as used herein means the net amount of a solute, typically a macromolecule, that has crossed a vascular bed and accumulated in the interstitium in response to a vascular permeabilizing agent or at a site of pathological angiogenesis [Nagy, J A, et al. Angiogenesis (2008) 11(2): 1009-119]. Vascular permeability by any measure is dramatically increased in acute and chronic inflammation, cancer, and wound healing. This hyperpermeability is mediated by acute or chronic exposure to vascular permeabilizing agents, particularly vascular permeability factor/vascular endothelial growth factor (VPF/VEGF, VEGF-A). Three distinctly different types of vascular permeability can be distinguished, based on the different types of microvessels involved, the composition of the extravasate, the anatomic pathways by which molecules of different size cross the vascular endothelium, the time course over which permeability is measured; and the animals and vascular beds that are being investigated. These are the basal vascular permeability (BVP) of normal tissues, the acute vascular hyperpermeability (AVH) that occurs in response to a single, brief exposure to VEGF-A or other vascular permeabilizing agents, and the chronic vascular hyperpermeability (CVH) that characterizes pathological angiogenesis [Nagy, J A, et al. Angiogenesis (2008) 11(2): 1009-119]

The term “vasculogenesis” as used herein refers to the process of new blood vessel formation.

The term “viroporin” as used herein refers to a family of small (about 100 amino acids or less) peptides that comprise one, two or three potential trans-membrane domains (TMDs) that can oligomerize to form an intact pore across the membrane of a cell by a process that is in the main mediated by hydrophobic interactions between hydrophobic integral membrane proteins. [Scott, C. & Griffin, S. J. General Virol. (2015) 96: 2000-27]. Viroporins can perform multiple functions during the virus life cycle, including those distinct from their role as oligomeric membrane channels. The viroporin family includes proteins encoded by many significant human pathogens including human immunodeficiency virus type I, picornaviruses (including poliovirus, Cocksackie virus, enterovirus 71, and human rhinovirus), alphaviruses (e.g., Chikungunya virus), paramyxoviruses (e.g., respiratory syncytial virus, mumps virus), orthomyxoviruses (e.g., influenza virus), Flativirus (e.g., dengue, virus, zika virus), coronavirus (E peptides, 3a protein), human papillomavirus (HPV), and numerous other RNA virus and DNA virus proteins. [Id.]

The term “wound healing” as used herein refers to the process by which the body repairs trauma to any of its tissues, especially those caused by physical means and with interruption of continuity.

The term “volume/volume percentage is a measure of the concentration of a substance in a solution. It is expressed as the ratio of the volume of the solute to the total volume of the solution multiplied by 100. Volume percent (vol/vol % or v/v %) should be used whenever a solution is prepared by mixing pure liquid solutions.

The term “weight by weight percentage” or wt/wt % is used herein to refer to the ratio of weight of a solute to the total weight of the solution.

### EMBODIMENTS

According to one aspect, the described invention provides a method for treating a subject at risk for a lung injury derived from a severe virus infection comprising

(a) receiving a subcutaneous injection of a bone marrow stimulant to mobilize CD34+ cells into the peripheral blood;

(b) harvesting CD34+ cells from the peripheral blood by apheresis;

(c) selecting CD34+ cells by positive selection;

(d) formulating a CLBS119 cell product by suspending the selected CD34+ cells in an isotonic solution with serum in concentrations ranging from 5% to 40% inclusive, i.e., 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40% and human serum albumin (HSA) in an amount ranging from 0.5% to 10%, inclusive, i.e., about 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 3.5%, 3.6%, 3.7%, 3.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%., 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, or about 10.% to form a pharmaceutical composition;

wherein the sterile pharmaceutical composition comprising a therapeutic amount of a mobilized nonexpanded, isolated population of autologous mononuclear cells enriched for CD34+ cells with a purity ranging from 55% to 100%, inclusive, i.e., 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, which further contains a subpopulation of potent CD34+/CXCR4+ cells; and

wherein, the mobilized nonexpanded, isolated population of autologous mononuclear cells enriched for CD34+ cells with a purity ranging from 55% to 100%, inclusive, i.e., 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, which further contains a subpopulation of potent CD34+/CXCR4+ cells when tested in vitro after passage through an infusion catheter after acquisition: (i) have CXCR-4 mediated chemotactic activity and move in response to SDF-1; (ii) can form hematopoietic colonies; and (iii) are at least 80% viable; and

(e) administering the cell product to the subject.

According to some embodiments, the serum is autologous serum. According to some embodiments, the serum is allogeneic AB negative serum. According to some embodiments, the amount of human serum albumin used as a substitute for serum can range from about 5% to about 20%, inclusive, i.e., about 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5% or about 20%.

According to some embodiments, imaging pathology of the lung injury includes the presence of one or more of ground glass nodules, patchy/punctate ground glass opacities, consolidation, increased density of the lung.

According to some embodiments, the severe lung injury comprises a pneumonia. According to some embodiments, the pneumonia includes one or more imaging findings comprising ground glass opacities, consolidation, crazy paving pattern, interlobular thickening, adjacent pleura thickening, and linear opacities.

According to some embodiments, the administering includes in vivo administration, as well as administration directly to tissue ex vivo. According to some embodiments, the administering is systemically (e.g., orally, buccally, parenterally, by inhalation or insufflation (i.e., through the mouth or through the nose), or rectally in dosage unit formulations containing conventional nontoxic pharmaceutically acceptable carriers, adjuvants, and vehicles as desired. According to some embodiments, the administering is parenterally. According to some embodiments, the administering is parenterally by intravenous infusion.

According to some embodiments, the method may modulate one or more outcomes selected from: pulmonary function; diffusing capacity of the lungs; oxygen saturation, inventory of COVID-19 related symptoms, radiographic evidence of pulmonary infiltrates; duration of use of oxygen, time to clinical improvement (TTCI), where clinical improvement is defined as the time from randomization to an improvement of two points (from the status at randomization) on a seven-category ordinal scale or live discharge from the hospital, whichever came first [Wang Y, et al. Comparative effectiveness of combined favipiravir and oseltamivir therapy versus oseltamivir monotherapy in critically ill patients with influenza virus infection. J Infect Dis, 2019] The seven-category ordinal scale consists of the following categories: 1) not hospitalized with resumption of normal activities, 2) not hospitalized, but unable to resume normal activities, 3) hospitalized, not requiring supplemental oxygen, 4) hospitalized, requiring supplemental oxygen, 5) hospitalized, requiring nasal high-flow oxygen therapy, noninvasive mechanical ventilation, or both, 6) hospitalized, requiring ECMO, invasive mechanical ventilation, or both, and 7) death; time to clinical recovery (TTCR), defined as the time (in hours) from initiation of study treatment until normalization of fever, respiratory rate, and oxygen saturation, and alleviation of cough, sustained for at least 72 hours. Normalization and alleviation criteria include: 1) Fever—≤38.3° C. oral, 2) Respiratory rate—≤24/minute on room air, 3) Oxygen saturation—>94% on room air, and 4) Cough—mild or absent on a subject reported scale of severe, moderate, mild, absent; length of time in ICU, length of time in hospital; or all-cause mortality, compared to a normal healthy control and a placebo control.

According to some embodiments, the method may modulate pulmonary function as measured by spirometry. A spirometer is a diagnostic device that measures the amount of air a subject is able to breathe in and out and the time it takes the subject to exhale completely after the subject has taken a deep breath. Interpretations of spirometry results require comparison between an individual's measured value and a reference value. Forced expiratory volume (FEV) measures how much air a person can exhale during a forced breath. The amount of air exhaled may be measured during the first (FEV1), second (FEV2), and/or third seconds (FEV3) of the forced breath. Forced vital capacity (FVC) is the total amount of air of air that can be forcibly exhaled from the lungs after taking the deepest breath possible, as measured by spirometry. If the FVC and the FEV1 are within 80% of the reference value, the results are considered normal. The normal value for the FEV1/FVC ratio is 70% (and 65% in persons older than age 65).

According to some embodiments, the method may modulate diffusing capacity of the lungs. To perform this test, a mask is placed over the subject's face. The subject takes in a deep breath of gas, holds his/her breath, and then the air exhaled is measured. The normal range for DLCO is as follows: 80-120% of its predicted value for men. 76-120% of its predicted value for women. Anemia, COPD with emphysema, interstitial lung disease (ILD), and pulmonary vascular diseases can decrease DLCO below the normal range. Although diffusion is often thought of as a function of alveolar membrane thickness, the dominant factor is usually the capillary blood volume, which influences both the surface area available for exchange and the volume of blood and hemoglobin available to accept carbon monoxide [Evans, S E et al. Chapter 9, Pulmonary Testing, in Clinical Respiratory Medicine, 3rd Ed. Spiro, S. et al. Mosby (2008)]. Asthma, obesity, and less commonly polycythemia, congestive heart failure, pregnancy, atrial septal defect, and hemoptysis or pulmonary hemorrhage can increase DLCO above the normal range. [Nguyen, L-P et al. Consultant (2016) 56(5)].

According to some embodiments, the method may modulate oxygen saturation as determined by pulse oximetry. Pulse oximetry measures how much oxygen the hemoglobin in the blood is carrying. This is called the oxygen saturation and is a percentage (scored out of 100). It uses a sensor placed on the fingertip or earlobe. The more the lungs are damaged, the more likely there is to be a problem with oxygen uptake.

According to some embodiments, the method may modulate one or more biomarkers selected from the group consisting of: neutrophil count and lymphocyte count, C-reactive protein (CRP); cell populations as assessed by flow cytometry; CXCR3+CD4+ T cells; CXCR3+CD8+ T cells, CXCR3+NK cells; level of tumor necrosis factor-alpha (TNF-α); IL-6, IL-10; troponin 1, or CXCL13 compared to a normal healthy control and a placebo control.

According to some embodiments, the method may modulate neutrophil count and lymphocyte count in blood. The normal range for the absolute neutrophil count (ANC) is 1.5 to 8.0 (1,500 to 8,000/mm³). In adults, a count of 1,500 neutrophils per microliter of blood or less is considered to be neutropenia. The normal lymphocyte range in adults is between 1,000 and 4,800 lymphocytes in 1 microliter (4) of blood.

According to some embodiments, the method may modulate a level of C-reactive protein (CRP) in blood: A high level of CRP in the blood is a marker of inflammation. For a standard CRP test, a normal reading is less than 10 milligram per liter (mg/L).

According to some embodiments, the method may modulate a level of CXCR3+CD4+ T cells in blood. CXCR3 is a chemokine receptor that is highly expressed on effector T cells and plays an important role in T cell trafficking and function. CXCR3 and its ligands regulate the migration of Th1 cells into sites of Th1-driven inflammation. Th1 cell-mediated inflammation is characterized by the recruitment of IFNγ producing CD4 T cells that normally mediate protection against intracellular pathogens. CXCR3 expression on effector T cells grants them entry into sites otherwise restricted. CXCR3 is rapidly induced on naïve cells following activation and preferentially remains highly expressed on Th1-type CD4+ T cells and effector CD8+ T cells [Groom, J R, and Luster, A D, Exp. Cell Res. (2011) 317 (5): 620-31].

According to some embodiments, the method may modulate a level of CXCR3+CD8+ T cells in blood. The chemokine receptor CXCR3 is involved in promoting CD8(+) T cell commitment to an effector fate rather than a memory fate by regulating T cell recruitment to an antigen/inflammation site. After systemic viral or bacterial infection, the contraction of CXCR3(−/−) antigen-specific CD8(+) T cells is significantly attenuated, resulting in massive accumulation of fully functional memory CD8(+) T cells. Early after infection, CXCR3(−/−) antigen-specific CD8(+) T cells fail to cluster at the marginal zone in the spleen where inflammatory cytokines such as IL-12 and IFN-α are abundant, thus receiving relatively weak inflammatory stimuli. Consequently, CXCR3(−/−) CD8(+) T cells exhibit transient expression of CD25 and preferentially differentiate into memory precursor effector cells as compared with wild-type CD8(+) T cells [Kurachi, M. et al. J. Exp. Med. (2011) 208 (8): 1605-20].

According to some embodiments, the method may modulate a level of CXCR3+NK cells in blood: Natural killer (NK) cells, innate lymphocytes with cytolytic activity against infected and transformed cells, are vital components of the antiviral immune response. Natural killer cell-mediated protection from infections requires efficacious NK cell recruitment to the sites of lymphocyte activation and infection. CXCR3 is known to be important in NK cell recruitment to the lung in homeostasis. NK cells are actively recruited to the lungs and airways during IAV infection. This recruitment is partially dependent upon CXCR3 and CCRS, respectively [Carlin, L E, et al. Front. Immunol. (2018) doi.org/10.3389/firmmu.2018.00781].

According to some embodiments, the method may modulate a level of tumor necrosis factor-alpha (TNF-α) in blood. TNF-α is a pro-inflammatory cytokine which can promote T cell apoptosis via interacting with its receptor, TNFR1, which expression is increased in aged T cells [Diaio, et al. Front. Immunol. (2020) doi.org/10.3389/firmmu.2020.0827, citing Aggarwal, S. et al. J. Immunol. (1999) 162: 2154-61; Gupta, S. et al. Cell Death Difer. (2005) 12: 177-83]. Tumor necrosis factor-α (TNF-α) and complement component 3 (C3) are two well-known pro-inflammatory molecules [Page, M. et al., Sci Rep. (2018) 8: 1812, citing Esmon, C T. Haemostasis (2000) 30 (2): 34-40]. They are not only upregulated in most inflammatory conditions, but their activities are closely linked. When TNF-α is upregulated, it contributes to changes in coagulation and C3 induction [Id., citing Liu, J. et al. J. Hepatol. (2015) 62: 354-362]. TNF-α plays a pivotal role in the disruption of macrovascular and microvascular circulation both in vivo and in vitro [Id., citing Zhang, H. et al., Clin. Sci. (Lond) (2009) 116: 219-30; Yamagishi, S. et al. Clinical Cardiol. (2009) 32: E29-E32], and is an important cytokine that can induce both apoptosis and inflammation [Id., citing Yang, G. & Shao, G F. Neurol. Sci. (2016) 37: 1253-59]. In the presence of reactive oxygen species (ROS), there is an increased production of TNF-α and, in turn, TNF-α signaling accentuates oxidative stress [Id., citing Zhang, H. et al. Clin. Sci. (Lond) (2009) 116: 219-30].

According to some embodiments, the method may modulate a level of interleukin-6 (IL-6) in blood. IL-6, when promptly and transiently produced in response to infections and tissue injuries, contributes to host defense through the stimulation of acute phase responses or immune reactions. Dysregulated and continual synthesis of IL-6 has been shown to play a pathological role in chronic inflammation and infection [Diaio, et al Front. Immunol. (2020) doi.org/10.3389/fimmu.2020.00827, citing Gaby, C. Arthritis Res. Ther. (2006) 8: S3; Jones, S A & Jenkins, B J. Nat. Rev. Immunol. (2018) 18: 773-89]. A study of 48 patients with COVID-19 admitted in China showed that the level of inflammatory cytokine IL-6 in critically ill patients increased significantly, almost 10 times that in other patients [Chen, X, et al., Clin. Infect. Dis. (2020) April 17: ciaa449]. The extremely high IL-6 level was closely correlated with the detection of SARS-CoV-2 viral load (RNAaemia) (R=0.902) and poor prognosis. The elevated IL-6 may be part of a larger cytokine storm which could worsen outcome.

According to some embodiments, the method may modulate a level of interleukin-10 (IL-10) in blood. IL-10, an inhibitory cytokine, not only prevents T cell proliferation, but also can induce T cell exhaustion. Blocking IL-10 function has been shown to successfully prevent T cell exhaustion in animal models of chronic infection [Diao, et al. Front. Immunol. (2020) doi.org/10.3389/fimmu.2020.00827, citing Brooks, D G, et al. Nat. Med. (2006) 12: 1301-9; Ejrnaes, M. et al., J. Exp. Med. (2006) 203: 2461-72]. T cell exhaustion is a state of T cell dysfunction that arises during many chronic infections and cancer that is defined by poor effector function, sustained expression of inhibitory receptors, and a transcriptional state distinct from that of functional effector or memory T cells [Id., citing McLane, L M, et al. Ann. Rev. Immunol. (2019) 37: 457-95]. Huang [Huang, C. et al. Lancet (2020) 395 497-506] showed that the levels of IL-2, IL-7, IL-10, TNF-α, G-CSF, IP-10, MCP-1, and MIP-1A were significantly higher in COVID-19 patients [Diao, et al Front. Immunol. (2020) doi.org/10.3389/fimmu.2020.00827] likewise reported that the levels of TNF-α, IL-6, and IL-10 were significantly increased in COVID-19 infected patients; statistical analysis illustrated that their levels in ICU patients were significantly higher than in Non-ICU patients. Within non-ICU patients, the concentration of IL-10, IL-6 and TNF-α was negatively correlated with total T cell count, CD4+ count and CD8+ count respectively. Diaio, et al. reported that T cells from COVID-19 patients had significantly higher levels of the exhausted marker PD-1. Increasing PD-1 and Tim-3 expression on T cells was seen as patients progressed from prodromal to overtly symptomatic stages. T cells may display limited function during prolonged infection as a result of exhaustion, which has been associated with the expression of these immune-inhibitory factors on the cell surface [Id., citing Wherry, E J et al. Nat. Rev. Immunol. (2015) 15: 486-99]. Counts of total T cells, CD8+ T cells or CD4+ T cells lower than 800, 300, or 400/μL, respectively, were negatively correlated with patient survival.

According to some embodiments, the method may modulate a level of troponin I in blood. Since the first data analyses in China, elevated cardiac troponin has been noted in a substantial proportion of patients, implicating myocardial injury as a possible pathogenic mechanism contributing to severe illness and mortality. Accordingly, high troponin levels are associated with increased mortality in patients with COVID-19. [Tersalvi, G. et al. J. Card. Fail. (2020) doi. org/10.1016/j. cardfail.2020. 04.009].

According to some embodiments, the method may modulate a level of CXCL13 in blood. CXC ligand 13 (CXCL13) [known as B cell attracting chemokine-1 (BCA-1) or B-lymphocyte chemoattractant (BLC)], is a potent chemoattractant for B lymphocytes; it induces a weak chemotactic response in T cells and macrophages and manifests no activity on neutrophils and monocytes. Expression of CXCR5, a G protein-coupled receptor originally isolated from Burkitt's lymphoma cells, is the specific receptor for BCA-1. Among cells of the hematopoietic lineages, the expression of CXCR5 is restricted to B lymphocytes and a subpopulation of T helper memory cells. BCA-1 is constitutively expressed in secondary lymphoid organs (e.g., spleen, lymph nodes, and Peyer's patches).

According to some embodiments, the administration parenterally by intravenous infusion is at a rate of infusion ranging from 0.5 to 2.0 mL/min, inclusive, i.e., 0.5, 0.6, 0.7, 0.8, 0.9, 1.0, 1.2, 1.3, 1.4, 1.5, 1.6, 1.7, 1.8, 1.9, 2.0 mL/min.

According to some embodiments, the therapeutic amount is an amount from about 50×10⁶ to 1000×10⁶ CD34+ cells, inclusive, i.e., about 50×10⁶, 51×10⁶, 52×10⁶, 53×10⁶, 54×10⁶, 55×10⁶, 56×10⁶, 57×10⁶, 58×10⁶, 59×10⁶, 60×10⁶, 61×10⁶, 62×10⁶, 63×10⁶, 64×10⁶, 65×10⁶, 66×10⁶, 67×10⁶, 68×10⁶, 69×10⁶, 70×10⁶, 71×10⁶, 72×10⁶, 73×10⁶, 74×10⁶, 75×10⁶, 76×10⁶, 77×10⁶, 78×10⁶, 79×10⁶, 80×10⁶, 81×10⁶, 82×10⁶, 83×10⁶, 84×10⁶, 85×10⁶, 86×10⁶, 87×10⁶, 88×10⁶, 89×10⁶, 90×10⁶, 91×10⁶, 92×10⁶, 93×10⁶, 94×10⁶, 95×10⁶, 96×10⁶, 97×10⁶, 98×10⁶, 99×10⁶, 100×10⁶; 110×10⁶, 120×10⁶, 130×10⁶, 140×10⁶, 150×10⁶, 160×10⁶, 170×10⁶, 180×10⁶, 190×10⁶, 200×10⁶, 210×10⁶, 220×10⁶, 230×10⁶, 240×10⁶, 250×10⁶, 260×10⁶, 270×10⁶, 280×10⁶, 290×10⁶, 300×10⁶, 310×10⁶, 320×10⁶, 330×10⁶, 340×10⁶, 350×10⁶, 360×10⁶, 370×10⁶, 380×10⁶, 390×10⁶, 400×10⁶, 410×10⁶, 420×10⁶, 430×10⁶, 440×10⁶, 450×10⁶, 460×10⁶, 470×10⁶, 480×10⁶, 490×10⁶, 500×10⁶ 510×10⁶, 520×10⁶, 530×10⁶, 540×10⁶, 550×10⁶, 560×10⁶, 570×10⁶, 580×10⁶, 590×10⁶, 600×10⁶, 610×10⁶, 620×10⁶, 630×10⁶, 640×10⁶, 650×10⁶, 660×10⁶, 670×10⁶, 680×10⁶, 690×10⁶, 700×10⁶; 710×10⁶, 720×10⁶, 730×10⁶, 740×10⁶, 750×10⁶, 760×10⁶, 770×10⁶, 780×10⁶, 790×10⁶, 800×10⁶, 810×10⁶, 820×10⁶, 830×10⁶, 840×10⁶, 850×10⁶, 860×10⁶, 870×10⁶, 880×10⁶, 890×10⁶, 900×10⁶; 910×10⁶, 920×10⁶, 930×10⁶, 940×10⁶, 950×10⁶, 960×10⁶, 970×10⁶, 980×10⁶, 990×10⁶, or 1000×10⁶, CD34+ cells.

According to some embodiments, the subpopulation of potent CD34+/CXCR4+ cells in the composition contains at least 0.1×10⁶ cells.

According to some embodiments, the subject at risk is a subject who has one or more predisposing factors to the development of lung injury following a severe virus infection. According to some embodiments, the predisposing factors include, without limitation, the very young, the elderly, those with pre-existing health conditions, such as chronic cardiopulmonary or renal disease; diabetes, immunosuppression, severe anemia, an existing illness, and those who are physically weak, e.g., due to malnutrition or dehydration. According to some embodiments, the subject at risk was diagnosed with COVID-19 (but no longer tests positive for active infection) and is currently hospitalized for treatment of pulmonary manifestations of the severe virus infection. According to some embodiments, the subject at risk received ventilative support during the severe virus infection. According to some embodiments, the subject at risk further displays cardiovascular complications, endothelial cell involvement across vascular beds. According to some embodiments, the subject at risk further comprises evidence for ongoing pulmonary involvement.

According to some embodiments, the subject at risk comprises biomarker evidence for ongoing inflammation. According to some embodiments, the biomarker evidence comprises elevated C-reactive protein; elevated troponin I or both.

According to some embodiments, the severe lung infection is caused by influenza or a human coronavirus. According to some embodiments, the human coronavirus is SARSCoV-2.

According to some embodiments, the lung injury comprises severe lung damage marked by one or more markers of inflammation, loss of lung endothelial cells/integrity and destruction of the lung microvasculature.

According to some embodiments, the subject at risk experiences acute respiratory failure. According to some embodiments, the acute respiratory failure comprises an acute lung injury. According to some embodiments, the acute lung injury comprises acute onset of diffuse bilateral pulmonary infiltrates by chest radiograph; a PaO₂/FiO₂≤300 and a pulmonary artery wedge pressure (PAWP)≤18.

According to some embodiments, the acute lung injury comprises one or more of acute inflammation, loss of alveolar-capillary membrane integrity, excessive transepithelial neutrophil migration, and release of pro-inflammatory mediators. According to some embodiments, the proinflammatory mediators include one or more of von Willebrand factor antigen, ICAM-1, SP-D, RAGE, IL-6, IL-8, TNFα, protein C, plasminogen activator inhibitor-1.

According to some embodiments, increased permeability of the epithelial membrane leads to an influx of protein-rich edema fluid into alveolar space.

According to some embodiments, upregulation of proinflammatory cytokines IL-6, IL-8 is indicative of acute lung injury.

According to some embodiments, the biomarkers alveolar epithelial biomarkers receptor for advanced glycation end-products (RAGE) and SP-D are biomarkers for lung epithelial injury.

According to some embodiments, an increase of IL-1β in serum is indicative of cell pyroptosis.

According to some embodiments, neutrophil elastase is a marker for excessive transepithelial neutrophil migration.

According to some embodiments, the acute lung injury progresses to acute respiratory distress syndrome comprising acute onset of diffuse bilateral pulmonary infiltrates by chest radiograph; a PaO₂/FiO₂≤200 and a pulmonary artery wedge pressure (PAWP)≤18 or no clinical evidence of left atrial hypertension.

According to some embodiments the acute respiratory failure comprises acute respiratory distress syndrome comprising one or more of diffuse alveolar damage (DAD), alveolar inflammation, or infiltration of neutrophils in the alveoli and distal bronchioles.

According to some embodiments, microvascular endothelial injury with increased release of vWf, upregulation of ICAM-1 or both is indicative of progression to increased capillary permeability.

According to some embodiments, the pharmaceutical composition may be efficacious to repair the lung injury, restore lung function, reduce scarring or fibrosis or a combination thereof.

According to some embodiments, the method may be efficacious to improve progression-free survival, overall survival or both.

According to some embodiments, the pharmaceutical composition may be efficacious to restore a CD34+ cell pool in the lung, lung vascular CD34+ cells, or both.

According to some embodiments, the pharmaceutical composition may attenuate the IL-6 and IL-8 inflammatory response associated with acute lung injury.

According to some embodiments the pharmaceutical composition may modulate platelet and neutrophil deposition, leukocyte accumulation or both in lung microvessels.

According to some embodiments, crosstalk between the CD34+ cells and the lung tissue may promote repair of the lung injury.

According to some embodiments, the repair derived from the CD34+ cells is a paracrine effect.

According to some embodiments, the paracrine effect is mediated by paracrine factors elaborated by the CD34+ cells.

According to some embodiments, the repair comprises reduced apoptosis of vascular endothelial cells, reduced apoptosis of lung endothelial cells, reduced apoptosis of lung epithelial cells; or increased angiogenesis.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and describe the methods and/or materials in connection with which the publications are cited.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1. Infarct-Related Artery Infusion of AMI-001

Preclinical studies describing characterization of AMI-001, a chemotactic hematopoietic stem cell product comprising a sterile pharmaceutical composition comprising. a nonexpanded, isolated population of autologous mononuclear cells derived from bone marrow enriched for CD34+ cells, which further contained a subpopulation of potent CD34+/CXCR4+ cells that, when tested in vitro after passage through a catheter after acquisition: (i) had CXCR-4 mediated chemotactic activity and moved in response to SDF-1; (ii) could form hematopoietic colonies; and (iii) were at least 70% viable, for infusion after ST elevation myocardial infarction is described in U.S. Pat. Nos. 7,794,705, 8,088,370, 8,637,005, 8,343,485, 8,425,899, 8,709,403, 9,034,316, 9,534,202, and 9,533,010. In brief, autologous CD34+ cells were harvested from bone marrow. CD34+ cells were selected from the harvested bone marrow by magnetic cell selection. If necessary, red blood cells were depleted by centrifugation. Samples of the CD34+ chemotactic hematopoietic stem cell product were removed and assayed for WBC count, Gram stain, and sterility. CD34+ cells were characterized by flow cytometry featuring CD34^{bright} and CD45^{dlm} fluorescence by double labeling with anti-CD34 and anti-CD45 antibodies (Beckman Coulter, P N IM3630). CD34+ cells and CD45+ cell viability was determined by excluding dying cells which take up the intercalating DNA dye 7-aminoactinomycin D (7AAD).

The chemotactic hematopoietic stem cell product that met the following criteria was released for intra-coronary infusion only if it was to be infused within about 48 hours to about 72 hours of completion of bone marrow harvest: CD34+ cell purity of at least about 70%, 75%, 80%, 85%, 90% or 95%; a negative Gram stain result for the selected positive fraction; endotoxin levels: less than about 0.5 endotoxin units/ml; viable CD34+ cell yield met the required dosing as per the treatment cohort; CD34+ cells were at least about 70%, 75%, 80%, 85%, 90% or 95% viable by 7-AAD; USP sterility result for “Positive Fraction Supernatant”: negative (14 days later); and bone marrow CD34+ cell selection was initiated within about 12 hours to about 24 hours of completion of bone marrow harvest. After meeting these release criteria, the chemotactic hematopoietic stern cell product was released for infusion and packaged for transportation to the catheterization facility. The chemotactic hematopoietic stem cell product was formulated in 10-mL of saline (0.9% Sodium Chloride, Injection, USP, Hospira, Cat#7983-09) supplemented with 1% HSA (Human Albumin USP, Alpha, Cat. #521303) (“Infusion Solution”) and a stabilizing amount of more than 10% autologous serum. Following release of the chemotactic hematopoietic stem cell product and cohort assignment, the chemotactic hematopoietic stem cell product was shipped to the catheterization site for direct infarct-related artery infusion (“intravascular administration”).

The series of preliminary preclinical studies described accomplished the following goals: (1) established optimization of the manufacturing process for the Mini bone-Marrow Harvest (MMH); (2) established the stability of the inbound MMH product and the outbound hematopoietic cell product; (3) established the internal diameter allowance and safety of the catheters; (4) established the compatibility of the cell product with the catheters used in the study; and (5) established the suitability of using the supernatant of the final hematopoietic cell product to represent the final hematopoietic cell product for stability testing.

The subpopulation of potent cells that (I) expressed CXCR-4 and (II) had CXCR-4 mediated chemotactic activity, expressed VEGFR-2 at very low levels (mean 0.84%, range 0 to 2.39%). Because the subpopulation of potent CD34+ cells co-expressed CXCR-4, {CXCR-4 co-expression; mean 60.63%, median 52% range 31-98% of CD34+ cells, capable of migrating in an SDF-1 gradient} while less than 2.5% of the CD34+ cells co-expresses VEGFR-2, functionally, these cells were VEGFR-2-, i.e., VEGFR-2 is not what drove the cells into the peri-infarct zone.

Studies showed that at 24 hours, 33 hours, 48 hours, 72 hours, and after 72 hours, the isolated CD34+ cells of the chemotactic hematopoietic stem cell product maintained 1) their viability, 2) their SDF-1/CXCR-4 mediated migratory ability, and 3) their ability to generate hematopoietic colonies in vitro equivalent to the 24 hour time point. Further studies showed that the CD34+ cells maintained their cell viability, growth in culture, and mobility in CXCR-4 assays as they passed through a catheter of 0.36 mm internal diameter.

Phase 1 Efficacy Data for infusion of the chemotactic hematopoietic stem cell product is described in U.S. Pat. Nos. 8,425,899, 9,034,316, 9,533,010, 9,534,202, and in Quyyumi, et al. Am. Heart J. (2011) 161: 98-105. Subjects selected for this study met all of the following clinical criteria (“inclusion criteria”):
Age: 18-75 years;
Acute ST segment elevation myocardial infarction meeting ACC/AHA criteria, with symptoms of chest pain within 3 days of admission. Criteria include (ST elevation>1 mm in limb leads or 2 mm in two or more precordial leads and increased levels of troponin, creatine kinase MB (CPK MB) or both), New York Heart Association (NYHA) heart failure class (to be recorded) of I, II or III;
Eligible for percutaneous coronary intervention (PCI);
Eligible for MRI;
Eligible for Single Proton Emission Computed Tomography (SPECT) imaging;
Subject must be able to provide informed written consent and must be willing to participate in all required study follow-up assessments;
Subjects must have a hemoglobin content (Hgb)>10 grams/dL, white blood cell count (WBC)>3500 cells/mm³, a platelet count>100,000 cells/mm³ and an international normalized ratio (INR, a blood coagulation test)<2.0 the day before the bone marrow collection;
Subjects must have a serum creatinine<2.5, total bilirubin<2.0 within 7 days of the bone marrow collection;
IRA and target lesion must be clearly identifiable when disease is present in more than one vessel;
Successful reperfusion and intracoronary stent placement, with Thrombolysis In Myocardial Infarction (TIMI) grade 2 or 3 flow, and infarct related artery (IRA) with <20% stenosis after revascularization;
Subjects must be deemed eligible to receive conscious sedation, mini-bone marrow harvest, and second catheterization for Chemotactic Hematopoietic Stem Cell Product infusion;
Included subjects must have an expected survival of at least one year and must not have multiple vessel disease after revascularization, or be expected to require intervention within 6 months of study entry.

, Subjects who satisfied any one of the following criteria did not qualify for, and were excluded from the study (“exclusion criteria”):
Subjects who are not candidates for percutaneous intervention, conscious sedation, MRI, SPECT imaging or mini-bone marrow harvest;
History of sustained chest pain unrelieved by nitrates, occurring 4 or more days before revascularization;
Subjects who fail to re-perfuse the infarct related coronary artery or to have successful stent placement;
Subjects presenting with cardiogenic shock (systolic pressure<80 on vasopressors or intraaortic counterpulsation);
Subjects with a side branch of the target lesion>2 mm and with ostial narrowing>50% diameter stenosis after revascularization;
Subjects unable to receive aspirin, clopidogrel or ticlopidine;
Subjects receiving warfarin must have an INR less than or equal to 2; the term INR refers to International Normalized Ratio, which is a system established by the World Health Organization (WHO) and the International Committee on Thrombosis and Hemostasis for reporting the results of blood coagulation (clotting) tests;
Subjects with severe aortic stenosis;
Subjects with severe immunodeficiency states (e.g., AIDS);
Subjects with cirrhosis requiring active medical management;
Subjects with malignancy requiring active treatment (except basal cell skin cancer);
Subjects with documented active alcohol and for other substance abuse;
Females of child bearing potential unless a pregnancy test is negative within 7 days of the mini-bone marrow harvest;
Subjects with ejection fractions greater than 50% on study entry by SPECT (96 to 144 hours after stent placement);
Subjects with less than three months of planned anti-platelet therapy post index procedure;
Subjects with multi vessel disease after revascularization requiring subsequent planned intervention during the next 6 months;
Subjects with participation in an ongoing investigational trial;
Subjects with active bacterial infection requiring systemic antibiotics.

As originally planned, there were to be four dosing cohorts (5 million, 10 million, 15 million and 20 million CD34+ cells) in the study. However more than 15 million cells post CD34+selection could not be obtained reliably. Therefore enrollment terminated at the end of cohort 3 with 15×10⁶ being the highest cell dose assessed. The cardiac performance measures Resting Total Severity Score (RTSS), percent infarct (“% Infarct”), End Systolic Volume (ESV) and Ejection Fraction (“EF”) were assessed at 3 months post treatment and at 6 months post treatment and compared with controls to assess efficacy of the compositions compared to controls. The data from Resting Total Severity Score represented cardiac perfusion, i.e., blood flow at the microvascular level, and muscle function.

Improvement in RTSS was seen only in subjects treated with 10×10⁶ or more CD34+ cells containing a subpopulation of at least 0.5×10⁶ potent CD34+ cells expressing CXCR-4 and having CXCR-4 mediated chemotactic activity. This dose therefore was the minimal therapeutically-effective dose.

The phase I study showed that after ST elevation myocardial infarction, infarct-related artery (IRA) infusion of at least 10×10⁶ autologous bone marrow-derived CD34+ cells formulated in phosphate buffered saline (PBS), 40% autologous human serum containing 1% human serum albumin and 25 USP U/mL of heparin sodium containing an enriched subpopulation of CD34+/CXCR-4+ SDF-1 mobile cells reduced cardiomyocyte cell death by improving perfusion, reduced apoptosis and preserved existing cardiomyocytes and their function in the infarct area. [Quyyumi, A A, et al., Am Heart J. (2011) 161: 98-105] The benefit imparted by infusion was through a paracrine and neoangiogenic effect, which affected immediate cell death and later changes consistent with ventricular remodeling.

### Example 2. Phase I/II Study, Autologous Peripheral Blood-Derived CD34+ Cells for Repair of COVID-19 Induced Pulmonary Damage

As with any invasive intervention being contemplated in hospitalized patients, a balance must be struck between the potential benefit of the intervention and the potential risk.

The entry criteria for this trial have been selected to identify patients who have suffered a severe injury to their lungs. The patients who meet the entry criteria for this study are at high risk for morbidity and mortality despite having acutely recovered. The ARDS literature tells us that these patients can expect up to 60% mortality and up to 40% will have a restrictive pattern on pulmonary function testing, indicating fibrosis [Chiumello, D. et al. Respiratory Care (2016) 61(5): 689-99]. While there are therapies that may have promise in the limitation of lung injury during an acute event, to our knowledge there is no human therapy currently being evaluated to reverse the damage that has already occurred. Accordingly, there is a significant unmet need.

Strong supportive evidence for the safety and efficacy of autologous CD34+ cell therapy in multiple tissue repair indications is described above. In terms of the risk of the therapy, safety data in hundreds of patients have been accumulated all of whom have extensive cardiovascular disease including critical limb ischemia and advanced heart failure. These patients have been able to tolerate the mobilization and collection procedure and the data has shown long-term benefit in treated vs control subjects.

Since the subjects of this study are receiving ventilator support or have recently been liberated from ventilator support, for this specific protocol the mobilization has been limited to a single administration of plerixafor, and a limited apheresis procedure will be performed.

Study Design

This is an open-label study in subjects who have been hospitalized due to infection with COVID-19 and have required mechanical ventilation due to respiratory failure. Subjects meeting the inclusion and none of the exclusion criteria will undergo a mobilization procedure with a single dose of plerixafor. Each subject will undergo apheresis to collect a mononuclear cell fraction which will be used for manufacturing CLBS119. Final CLBS119 product will be returned to the clinician on the morning of the second day after apheresis and will be administered to the subject by intravenous infusion the same day. Subjects will be assessed prior to treatment and following treatment for pulmonary function, disease status, and exploratory biomarkers with follow-up through 6 months after discharge.

Number of subjects: 12.

Clinical indication: infection with SARS-CoV-2.

Subject participation will primarily be during the hospitalization period, generally a few weeks, plus follow-up after discharge. Total participation is expected to be 7 to 8 months.

Screening Phase

To be eligible, subjects will already have been diagnosed with COVID-19, and are currently hospitalized for treatment of pulmonary manifestations. Subjects may undergo screening to establish eligibility. Once voluntary consent has been obtained and eligibility criteria have been verified, the subject can proceed to the treatment phase. Subjects who are not yet eligible but expected to become eligible may be asked to consent and begin the screening process. All such subjects will continue to receive best available care. Records must be kept of all subjects consented and all screening procedures performed under this protocol.

Up to 12 subjects will be treated with CLBS119 under this protocol.

Inclusion Criteria

Subjects who meet ALL of the following criteria are eligible for this study:
(1) Men or women age ≥18;
(2) Initial diagnosis with COVID-19 based on PCR test;
(3) Hospitalized for COVID-19;
(4) Required ventilatory support for COVID-19 pneumonia/ARDS;
(5) Evidence for ongoing pulmonary involvement on physical exam, chest X-ray, or chest CT;
(6) COVID-19 viral clearance documented by conversion to negative PCR test;
(7) If subject is of childbearing potential, the subject must have a negative pregnancy test at screening;
(8) Subject is willing and able to comply with the requirements of the protocol; and
(9) Able to provide signed informed consent.

Exclusion Criteria:

Subjects who meet ANY ONE of the following criteria are ineligible for this study:
1. Immunocompromised or use of immunosuppressive agents other than corticosteroids;
2. History of autoimmune disease;
3. Evidence of multiorgan failure;
4. Subject has a known allergy to mouse proteins;
5. Subject tests positive for human immunodeficiency virus (HIV), hepatitis B or hepatitis C;
6. Recent history of abuse or current abuser of alcohol or recreational drugs;
7. Subject is pregnant or lactating at the time of signing the consent;
8. Malignant neoplasm (other than adequately treated non-melanoma skin cancer or in situ cervical carcinoma) within 5 years prior to screening;
9. Participation in any other clinical trial of an experimental treatment for COVID-19;
10. History of sickle cell disease; or
11. Any other condition which, in the opinion of the investigator, may preclude the subject from safe participation in the study or compromise data integrity.

Investigational Product CLBS119

This autologous CD34+ cell product comprising CD34+ cells are isolated from mobilized peripheral blood.

The process for obtaining autologous CD34+ cells is as follows.

All eligible research subjects will receive a subcutaneous injection of a bone marrow stimulant/hematopoietic stem cell mobilizer Mozobil® (plerixafor) at a dose of 240 μg/kg to mobilize CD34+ cells into the peripheral blood. Approximately 10 to 12 hours later, a sample of blood for assessment of CD34+ cell counts in peripheral blood will be taken for analysis at the manufacturing site. Subjects will then undergo apheresis to collect CD34+ cells.

Apheresis procedure for harvesting CD34+ cells from peripheral blood

Apheresis will be planned to occur approximately 8 to 10 hours following administration of plerixafor. Just prior to apheresis, a blood sample will be collected so that CD34+ cell counts can be assessed. Subjects will then undergo apheresis with 4 Total Blood Volumes (TBV) of whole blood processed to obtain the autologous mononuclear cell product. Blood is collected to provide autologous serum, which can be used for formulating the CLBS119 final product. Alternatively, allogeneic AB negative serum can be used, or human serum albumin ranging from 5% to 20%, inclusive can be used as a substitute for serum.

Immediately after apheresis, the cell product and blood will be sent to a centralized facility where autologous CD34+ cells will be selected using the CliniMACS System (Miltenyi Biotec, Bergisch Gladbach, Germany).

Release specifications for CLBS119 CD34+ cell product are shown in table 1.

TABLE 1
Release specifications, CLSB CD34+ cell product
Specification
≥70% CD34+ cell purity as determined by flow cytometry
≥80% CD34+ cell viability as determined by flow cytometry
Maximum dose: 500 × 10⁶ CD34+ cells
Endotoxin ≤5 EU/kg*
Negative microbial presence by Gram Stain*
Negative microbial growth**
CLBS119 CD34+ Cell Product expiry ≤90 hours from HPC-
Apheresis collection
*Endotoxin and gram stain testing will begin immediately after manufacturing. I both tests are negative, the product will be conditionally released for transplant.
**Sterility testing (<USP71>) will begin immediately after manufacturing. The sterility testing will be carried out for a total of 14 days, after which point, final release will occur.

A three step release process will be used for the CLBS119 CD34+ Cell Product.

Step 1: the final cell product will be released for shipment under quarantine status after meeting the release criteria of dose, CD34+ cell viability and purity.

Step 2: For safety release testing, an aliquot of the CLBS119 CD34+ Cell Product will be taken for endotoxin and Gram stain testing. Additionally, sterility testing (<USP71>) will be performed using the CLBS119 CD34+ Cell Product. The final product will be conditionally released by the manufacturer's QA staff for infusion after negative results have been obtained for endotoxin and Gram stain. The manufacturer's QA staff will notify the clinical site that the product is conditionally released for infusion. The final product must be infused within 90 hours of the completion of the apheresis collection.

Step 3. Final sterility results will not be available at the time of product release for administration. The final release occurs after completion of sterility testing, which takes 14 days to perform. If the test is positive, the principal investigator and study coordinator will be notified immediately, provided with identification of the organism once available, the results will be reviewed by the principal investigator in consultation with an infectious disease specialist and decisions regarding treatment and repeat testing will be based on these consultations.

A two-step release process will be used for the placebo, which is the same described in step 2 and 3 of the CLBS119 CD34+ cell product.

Dosage form: Solution/Suspension, Dosage: up to 500×10⁶ CD34+ cells in a volume of 10 mL

Packaging, Labeling and Storage.

Autologous CD34+ cells will be suspended in an isotonic solution with autologous or allogeneic AB negative serum ranging from 5% to 40%, inclusiveLand human serum albumin (0.5-10% with serum; 5-20% as a substitute for serum) and sealed in a labeled sterile bag. The label will include subject identifiers, product expiration date & time, product volume, product identifier, temperature requirements, contact information, processing site information, and applicable cautions and warnings. The cell product bag is placed in secondary absorbent packaging and then in a secure transportation box (temperature maintained at 2-10° C.) to be delivered to the investigative site, usually the second day after apheresis.

Upon arrival at the clinical site, clinical trial personnel from the site will open the shipping container containing CLBS119 and record time of unpacking. The temperature reading from the temperature recording device will be recorded. If the temperature reading is outside of the 2-10° C. range, site personnel must contact Caladrius to determine whether the investigational product can be administered, and temperature deviation information must be documented. CLBS119 can remain at ambient temperature for up to 4 hours prior to administration; however, if there will be a delay of more than 4 hours before administration of the Investigational Product, the Sponsor will be contacted for storage and administration instructions.

Administration

Upon notification from the cell processing facility that the CLBS119 product has been released for infusion, the subject will receive CLBS119 by intravenous infusion. The subject should be monitored during infusion for any signs of adverse effects. Following infusion, the subject should receive standard post-infusion care, including observation of the infusion site, monitoring of vital signs, and assessment of adverse events.

Treatment Phase

Treatment of an individual subject should not be undertaken if any issue is identified which would create an unreasonable risk for administration of CLBS119. Treatment should also not be undertaken if any issue is identified which would create an unreasonable risk for the testing required under the protocol. Any such decision may be made prior to initiation of the treatment procedure or at any point during CLBS119 administration procedure. Subjects not treated with CLBS119 may be replaced at the discretion of the Sponsor.

Each subject will receive the maximum dose that can be manufactured, after removal of cells for testing, up to a limit of 200 or 500×10⁶ CD34+ cells, by intravenous infusion. The total product volume will be administered at a rate of up to 2.0 mL/min. The dose level of up to 300×10⁶ cells by intracoronary infusion was used in a previous study CLBS16-P01 for coronary microvascular dysfunction and was found to be safe and effective. Since in this protocol, the cells are being administered by intravenous infusion it is logical to extend the dose window, and up to 500×10⁶ cells was chosen. The minimum dose to be delivered will be 0.5×10⁶ potent CD34+CXCR4+ cells.

Dosage frequency: Once

Follow-Up Phase

Safety and efficacy assessments will be performed daily for the first 5 days after treatment and every other day after that until discharge from the hospital. Subjects will be followed-up through 6 months after discharge.

Once treated with investigational product, a subject should be followed for all safety and efficacy measures outlined in the protocol, to the extent possible. However, efficacy measures such as pulmonary function testing should not be undertaken if any issue is identified which would create an unreasonable risk for the subject.

Duration of Subject Participation

Duration of subject participation will be up to approximately 8 months.

Suspending Enrollment or Stopping the Study:

The investigators and the medical monitor for this study will review adverse events on an ongoing basis and will communicate with each other if any evolving safety signal is perceived. In the event that an evolving safety signal is perceived, the medical monitor may choose to suspend enrollment while the safety events are investigated or to halt treatment of further subjects. In either case, observation of subjects already treated should continue to the extent possible.

Randomization and Blinding

As a single-arm study, there will be no randomization and no blinding. In the context of a relatively small sample size and open-label design we will rely on several categories of data to collect evidence for bioactivity.

Clinical Outcomes

In addition to safety/adverse event monitoring we will assess time to complete recovery and return to normal function

Lung Function

Measurements of pulmonary function and lung diffusion capacity are sensitive markers of lung recovery and will be monitored before and after treatment.

Lung imaging will be performed to monitor/document resolution of infiltrates.

Biomarkers

An evolving array of biomarkers has been used to monitor COVID-19 patients, including markers of lung injury and inflammation. Many biomarkers have been included below, but the state of biomarker knowledge will be reassessed immediately prior to enrolling the first patient to insure that all potentially informative markers have been have included.

Safety Endpoints include:
Adverse events [AEs]
Laboratory investigations
Physical examinations
Vital signs
Death

Efficacy Endpoints include:
Change in pulmonary function as assessed by spirometry.
Diffusing capacity of the lungs (DLCO), meaning diffusion across the lungs of carbon monoxide:
Change in oxygen saturation by pulse oximeter:
Inventory of COVID-19 related symptoms
Change in radiographic evidence of pulmonary infiltrates
Duration of use of oxygen
Time to clinical improvement (TTCI), where clinical improvement is defined as the time from randomization to an improvement of two points (from the status at randomization) on a seven-category ordinal scale or live discharge from the hospital, whichever came first [Wang Y, et al. Comparative effectiveness of combined favipiravir and oseltamivir therapy versus oseltamivir monotherapy in critically ill patients with influenza virus infection. J Infect Dis, 2019] The seven-category ordinal scale consists of the following categories: 1) not hospitalized with resumption of normal activities, 2) not hospitalized, but unable to resume normal activities, 3) hospitalized, not requiring supplemental oxygen, 4) hospitalized, requiring supplemental oxygen, 5) hospitalized, requiring nasal high-flow oxygen therapy, noninvasive mechanical ventilation, or both, 6) hospitalized, requiring ECMO, invasive mechanical ventilation, or both, and 7) death.
Time to clinical recovery (TTCR), defined as the time (in hours) from initiation of study treatment until normalization of fever, respiratory rate, and oxygen saturation, and alleviation of cough, sustained for at least 72 hours. Normalization and alleviation criteria: 1) Fever—≤38.3° C. oral, 2) Respiratory rate—≤24/minute on room air, 3) Oxygen saturation—>94% on room air, and 4) Cough—mild or absent on a subject reported scale of severe, moderate, mild, absent
Length of time in ICU
Length of time in hospital
All-cause mortality

Exemplary Biomarker Endpoints include, without limitation:
Change in neutrophil count and lymphocyte count.
Change in C-reactive protein (CRP).
Change in cell populations as assessed by flow cytometry
   CXCR3+CD4+ T cells
   CXCR3+CD8+ T cells
   CXCR3+NK cells
Change in tumor necrosis factor-alpha (TNF-α):
Change in interleukin-6 (IL-6)
Change in interleukin-10 (IL-10)
Change in troponin I
Change in CXCL13: CXC ligand 13 (CXCL13) [known as B cell attracting chemokine-1 (BCA-1) or B-lymphocyte chemoattractant (BLC)]

Statistics.

Power Estimate—No formal sample size calculation was performed for this study. With a sample size of 12 subjects, if the incidence rate for an AE is 2% or 5%, the probability of observing the event in at least one subject during the study is approximately 21% or 46%.

Planned Statistical Analysis—All AEs will be coded using Medical Dictionary for Regulatory Activities (MedDRA), A TEAE is defined as an AE that starts or worsens on or after study Day 1. The number and percentage of subjects with TEAEs will be summarized by MedDRA system organ class, high level term, and preferred term overall, by severity and by relationships to study drug.

Descriptive statistics will be presented for each of the efficacy endpoints.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A method for treating a subject at risk for a lung injury derived from a severe virus infection comprising
(a) receiving a subcutaneous injection of a bone marrow stimulant to mobilize CD34+ cells into the peripheral blood;
(b) harvesting CD34+ cells from the peripheral blood by apheresis;
(c) selecting CD34+ cells by positive selection;
(d) formulating a CLBS119 cell product by suspending the selected CD34+ cells in an isotonic solution with serum ranging from 5% to 40%, inclusive, and human serum albumin ranging from 0.5-10%, inclusive, to form a pharmaceutical composition; and
(e) administering the cell product to the subject;
wherein the sterile pharmaceutical composition comprising a therapeutic amount of a mobilized nonexpanded, isolated population of autologous mononuclear cells enriched for CD34+ cells with purity ranging from 55% to 100%, inclusive, which further contains a subpopulation of potent CD34+/CXCR4+ cells; and
wherein, the mobilized nonexpanded, isolated population of autologous mononuclear cells enriched for CD34+ cells with purity ranging from 55% to 100%, inclusive, which further contains a subpopulation of potent CD34+/CXCR4+ cells when tested in vitro after passage through an infusion catheter after acquisition: (i) has CXCR-4 mediated chemotactic activity and moves in response to SDF-1; (ii) can form hematopoietic colonies; and (iii) is at least 80% viable.

2. The method according to claim 1, wherein
(a) the serum is autologous serum or allogeneic AB negative serum; or
(b) in the absence of serum, from 5% to 20%, inclusive human serum albumin can substitute for serum; or
(c) the lung injury comprises severe lung damage marked by one or more of inflammation, loss of lung endothelial cells/integrity and destruction of the lung microvasculature; or
(d) the administering is by infusion, and rate of infusion ranges from 0.5 to 2.0 mL/min; or
(e) the therapeutic amount is an amount ranging from about 50×10⁶, to about 1000×10⁶ inclusive, i.e., 51×10⁶, 52×10⁶, 53×10⁶, 54×10⁶, 55×10⁶, 56×10⁶, 57×10⁶, 58×10⁶, 59×10⁶, 60×10⁶, 61×10⁶, 62×10⁶, 63×10⁶, 64×10⁶, 65×10⁶, 66×10⁶, 67×10⁶, 68×10⁶, 69×10⁶, 70×10⁶, 71×10⁶, 72×10⁶, 73×10⁶, 74×10⁶, 75×10⁶, 76×10⁶, 77×10⁶, 78×10⁶, 79×10⁶, 80×10⁶, 81×10⁶, 82×10⁶, 83×10⁶, 84×10⁶, 85×10⁶, 86×10⁶, 87×10⁶, 88×10⁶, 89×10⁶, 90×10⁶, 91×10⁶, 92×10⁶, 93×10⁶, 94×10⁶, 95×10⁶, 96×10⁶, 97×10⁶, 98×10⁶, 99×10⁶, 100×10⁶; 110×10⁶, 120×10⁶, 130×10⁶, 140×10⁶, 150×10⁶, 160×10⁶, 170×10⁶, 180×10⁶, 190×10⁶, 200×10⁶, 210×10⁶, 220×10⁶, 230×10⁶, 240×10⁶, 250×10⁶, 260×10⁶, 270×10⁶, 280×10⁶, 290×10⁶, 300×10⁶, 310×10⁶, 320×10⁶, 330×10⁶, 340×10⁶, 350×10⁶, 360×10⁶, 370×10⁶, 380×10⁶, 390×10⁶, 400×10⁶, 410×10⁶, 420×10⁶, 430×10⁶, 440×10⁶, 450×10⁶, 460×10⁶, 470×10⁶, 480×10⁶, 490×10⁶, 500×10⁶ 510×10⁶, 520×10⁶, 530×10⁶, 540×10⁶, 550×10⁶, 560×10⁶, 570×10⁶, 580×10⁶, 590×10⁶, 600×10⁶, 610×10⁶, 620×10⁶, 630×10⁶, 640×10⁶, 650×10⁶, 660×10⁶, 670×10⁶, 680×10⁶, 690×10⁶, 700×10⁶; 710×10⁶, 720×10⁶, 730×10⁶, 740×10⁶, 750×10⁶, 760×10⁶, 770×10⁶, 780×10⁶, 790×10⁶, 800×10⁶, 810×10⁶, 820×10⁶, 830×10⁶, 840×10⁶, 850×10⁶, 860×10⁶, 870×10⁶, 880×10⁶, 890×10⁶, 900×10⁶; 910×10⁶, 920×10⁶, 930×10⁶, 940×10⁶, 950×10⁶, 960×10⁶, 970×10⁶, 980×10⁶, 990×10⁶, or 1000×10⁶ CD34+ cells; or
(f) the subpopulation of potent CD34+/CXCR4+ cells in the composition contains at least 0.1×10⁶ cells.

3. The method according to claim 1, wherein the method modulates one or more outcomes selected from: pulmonary function; diffusing capacity of the lungs; oxygen saturation, inventory of COVID-19 related symptoms, radiographic evidence of pulmonary infiltrates; duration of use of oxygen, time to clinical improvement (TTCI), time to clinical recovery (TTCR), length of time in ICU, length of time in hospital; or all-cause mortality, compared to a normal healthy control and a placebo control.

4. The method according to claim 1, wherein the subject at risk is a subject who has one or more predisposing factors to the development of lung injury following a severe virus infection.

5. The method according to claim 4, wherein the predisposing factors include the very young, the elderly, those with pre-existing health conditions, such as chronic cardiopulmonary or renal disease; diabetes, immunosuppression, severe anemia, an existing illness, and those who are physically weak.

6. The method according to claim 1, wherein
(a) the subject at risk was diagnosed with COVID-19 and is currently hospitalized for treatment of pulmonary manifestations of the severe virus infection; or
(b) the subject at risk received ventilative support during the severe virus infection; or
(c) the subject at risk further displays cardiovascular complications; or
(d) the subject at risk further comprises evidence for ongoing pulmonary involvement; or
(e) the subject at risk comprises biomarker evidence for ongoing inflammation.

7. The method according to claim 6, wherein the biomarker evidence comprises a modulated level of one or more of C-reactive protein; troponin, white blood cell count;
lymphocyte count; lactate dehydrogenase; tumor necrosis factor alpha; IL-1, IL-6, IL-12, one or more interferon(s), compared to a normal healthy control or a control that has not been treated with the cell product.

8. The method according to claim 4, wherein the severe lung infection is caused by influenza or a human coronavirus.

9. The method according to claim 8, wherein the human coronavirus is SARSCoV-2.

10. The method according to claim 1, wherein the lung injury comprises acute respiratory failure.

11. The method according to claim 10, wherein the acute respiratory failure comprises an acute lung injury or acute respiratory distress syndrome.

12. The method according to claim 11,
(a) wherein the acute lung injury comprises acute onset of diffuse bilateral pulmonary infiltrates by chest radiograph; a PaO₂/FiO₂≤300 and a pulmonary artery wedge pressure (PAWP)≤18; or
(b) wherein the acute lung injury comprises one or more of acute inflammation, loss of alveolar-capillary membrane integrity, excessive transepithelial neutrophil migration, and release of pro-inflammatory mediators; or
(c) the acute respiratory distress syndrome comprises acute onset of diffuse bilateral pulmonary infiltrates by chest radiograph; a PaO₂/FiO₂≤200 and a pulmonary artery wedge pressure (PAWP)≤18 crosstalk between the CD34+ cells and the lung tissue promotes repair of the lung injury; or
(d) the proinflammatory mediators include one or more of von Willebrand factor (vWf) antigen, intracellular adhesion molecule-1 (ICAM-1), surfactant protein D (SP-D), receptor for advanced glycation end-products (RAGE), IL-6, IL-8, TNF-α, protein C, or plasminogen activator inhibitor-1; or
(e) the acute respiratory distress syndrome comprises one or more of diffuse alveolar damage (DAD), alveolar inflammation, or infiltration of neutrophils in the alveoli and distal bronchioles.

13. The method according to claim 12, wherein RAGE and SP-D are biomarkers for lung epithelial injury.

14. The method according to claim 12, wherein neutrophil elastase is a marker for excessive transepithelial neutrophil migration.

15. The method according to claim 12, wherein a microvascular endothelial injury with increased release of vWf antigen, upregulation of ICAM-1 or both is indicative of progression to increased capillary permeability.

16. The method according to claim 1, wherein
(a) the pharmaceutical composition is efficacious to repair the lung injury, restore lung function, reduce scarring or fibrosis or a combination thereof; or
(b) the method is efficacious to improve progression-free survival, overall survival or both; or.
(c) the pharmaceutical composition is efficacious to restore a CD34+ cell pool in the lung, lung vascular CD34+ cells, or both; or
(d) the pharmaceutical composition attenuates the IL-6 and IL-8 inflammatory response associated with acute lung injury; or
(e) the pharmaceutical composition modulates platelet and neutrophil deposition, leukocyte accumulation in lung microvessels.

17. The method according to claim 1, wherein
(a) the pharmaceutical composition modulates platelet and neutrophil deposition, leukocyte accumulation in lung microvessels; or
(b) crosstalk between the CD34+ cells and the lung tissue promotes repair of the lung injury.

18. The method according to claim 17, wherein the crosstalk is a paracrine effect.

19. The method according to claim 18, wherein the paracrine effect is mediated by paracrine factors elaborated by the CD34+ cells.

20. The method according to claim 17, wherein the repair comprises reduced apoptosis of vascular endothelial cells, lung endothelial cells, or lung epithelial cells, or increased angiogenesis or both.
